SEQUENCE LISTING (31 sequences)

SEQ ID NO: 1 - protein, 264 aa, Artificial sequence
  Homo sapiens
HGDGSFSDEMNTILDNLAARDFINWLIQTKITDGGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 284 aa, Artificial sequence
  Homo sapiens
METPAQLLFLLLLWLPDTTGHGDGSFSDEMNTILDNLAARDFINWLIQTKITDGGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - DNA, 852 bp, Artificial sequence
  Homo sapiens
atggaaaccccggcgcagctgctgtttctgctgctgctgtggctgccggataccaccggccatggcgatggcagctttagcgatgaaatgaacaccattctggataacctggcggcgcgcgattttattaactggctgattcagaccaaaattaccgatggcggcggcggcggcgataaaacccatacctgcccgccgtgcccggcgccggaagcggcgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggc

SEQ ID NO: 4 - protein, 265 aa, Artificial sequence
  Homo sapiens
HGDGSFSDEMNTILDNLAARDFINWLIQTKITDGGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 285 aa, Artificial sequence
  Homo sapiens
METPAQLLFLLLLWLPDTTGHGDGSFSDEMNTILDNLAARDFINWLIQTKITDGGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 855 bp, Artificial sequence
  Homo sapiens
atggaaaccccggcgcagctgctgtttctgctgctgctgtggctgccggataccaccggccatggcgatggcagctttagcgatgaaatgaacaccattctggataacctggcggcgcgcgattttattaactggctgattcagaccaaaattaccgatggcggcggcggcggcgataaaacccatacctgcccgccgtgcccggcgccggaagcggcgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 7 - protein, 274 aa, Artificial sequence
  Homo sapiens
HGDGSFSDEMNTILDNLAARDFINWLIQTKITDGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 294 aa, Artificial sequence
  Homo sapiens
METPAQLLFLLLLWLPDTTGHGDGSFSDEMNTILDNLAARDFINWLIQTKITDGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 9 - DNA, 882 bp, Artificial sequence
  Homo sapiens
atggaaaccccggcgcagctgctgtttctgctgctgctgtggctgccggataccaccggccatggcgatggcagctttagcgatgaaatgaacaccattctggataacctggcggcgcgcgattttattaactggctgattcagaccaaaattaccgatggcggcggcggcagcggcggcggcggcagcggcggcggcggcagcgataaaacccatacctgcccgccgtgcccggcgccggaagcggcgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggc

SEQ ID NO: 10 - protein, 275 aa, Artificial sequence
  Homo sapiens
HGDGSFSDEMNTILDNLAARDFINWLIQTKITDGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 295 aa, Artificial sequence
  Homo sapiens
METPAQLLFLLLLWLPDTTGHGDGSFSDEMNTILDNLAARDFINWLIQTKITDGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - DNA, 885 bp, Artificial sequence
  Homo sapiens
atggaaaccccggcgcagctgctgtttctgctgctgctgtggctgccggataccaccggccatggcgatggcagctttagcgatgaaatgaacaccattctggataacctggcggcgcgcgattttattaactggctgattcagaccaaaattaccgatggcggcggcggcagcggcggcggcggcagcggcggcggcggcagcgataaaacccatacctgcccgccgtgcccggcgccggaagcggcgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggcaaa

SEQ ID NO: 13 - protein, 259 aa, Artificial sequence
  Homo sapiens
HGDGSFSDEMNTILDNLAARDFINWLIQTKITDDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 279 aa, Artificial sequence
  Homo sapiens
METPAQLLFLLLLWLPDTTGHGDGSFSDEMNTILDNLAARDFINWLIQTKITDDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - DNA, 837 bp, Artificial sequence
  Homo sapiens
atggaaaccccggcgcagctgctgtttctgctgctgctgtggctgccggataccaccggccatggcgatggcagctttagcgatgaaatgaacaccattctggataacctggcggcgcgcgattttattaactggctgattcagaccaaaattaccgatgataaaacccatacctgcccgccgtgcccggcgccggaagcggcgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggc

SEQ ID NO: 16 - protein, 276 aa, Artificial sequence
  Homo sapiens
HGDGSFSDEMNTILDNLAARDFINWLIQTKITDGGGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 17 - protein, 296 aa, Artificial sequence
  Homo sapiens
METPAQLLFLLLLWLPDTTGHGDGSFSDEMNTILDNLAARDFINWLIQTKITDGGGGGGSGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 18 - DNA, 891 bp, Artificial sequence
  Homo sapiens
atggaaaccccggcgcagctgctgtttctgctgctgctgtggctgccggataccaccggccatggcgatggcagctttagcgatgaaatgaacaccattctggataacctggcggcgcgcgattttattaactggctgattcagaccaaaattaccgatggcggcggcggcggcggcagcggcggcggcggcagcggcggcggcggcagcgcggataaaacccatacctgcccgccgtgcccggcgccggaagcggcgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggc

SEQ ID NO: 19 - protein, 316 aa, Artificial sequence
  Homo sapiens
HGDGSFSDEMNTILDNLAARDFINWLIQTKITDGAPGGGGGAAAAAGGGGGGAPGGGGGAAAAAGGGGGGAPGGGGGAAAAAGGGGGGAPDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 336 aa, Artificial sequence
  Homo sapiens
METPAQLLFLLLLWLPDTTGHGDGSFSDEMNTILDNLAARDFINWLIQTKITDGAPGGGGGAAAAAGGGGGGAPGGGGGAAAAAGGGGGGAPGGGGGAAAAAGGGGGGAPDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 21 - DNA, 1008 bp, Artificial sequence
  Homo sapiens
atggaaaccccggcgcagctgctgtttctgctgctgctgtggctgccggataccaccggccatggcgatggcagctttagcgatgaaatgaacaccattctggataacctggcggcgcgcgattttattaactggctgattcagaccaaaattaccgatggcgcgccgggcggcggcggcggcgcggcggcggcggcgggcggcggcggcggcggcgcgccgggcggcggcggcggcgcggcggcggcggcgggcggcggcggcggcggcgcgccgggcggcggcggcggcgcggcggcggcggcgggcggcggcggcggcggcgcgccggataaaacccatacctgcccgccgtgcccggcgccggaagcggcgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggc

SEQ ID NO: 22 - protein, 266 aa, Artificial sequence
  Homo sapiens
HGDGSFSDEMNTILDNLAARDFINWLIQTKITDGGGGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 286 aa, Artificial sequence
  Homo sapiens
METPAQLLFLLLLWLPDTTGHGDGSFSDEMNTILDNLAARDFINWLIQTKITDGGGGGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - DNA, 858 bp, Artificial sequence
  Homo sapiens
atggaaaccccggcgcagctgctgtttctgctgctgctgtggctgccggataccaccggccatggcgatggcagctttagcgatgaaatgaacaccattctggataacctggcggcgcgcgattttattaactggctgattcagaccaaaattaccgatggcggcggcggcggcggcggcgataaaacccatacctgcccgccgtgcccggcgccggaagcggcgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggc

SEQ ID NO: 25 - protein, 269 aa, Artificial sequence
  Homo sapiens
HGDGSFSDEMNTILDNLAARDFINWLIQTKITDGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 289 aa, Artificial sequence
  Homo sapiens
METPAQLLFLLLLWLPDTTGHGDGSFSDEMNTILDNLAARDFINWLIQTKITDGGGGSGGGGSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 27 - DNA, 867 bp, Artificial sequence
  Homo sapiens
atggaaaccccggcgcagctgctgtttctgctgctgctgtggctgccggataccaccggccatggcgatggcagctttagcgatgaaatgaacaccattctggataacctggcggcgcgcgattttattaactggctgattcagaccaaaattaccgatggcggcggcggcagcggcggcggcggcagcgataaaacccatacctgcccgccgtgcccggcgccggaagcggcgggcggcccgagcgtgtttctgtttccgccgaaaccgaaagataccctgatgattagccgcaccccggaagtgacctgcgtggtggtggatgtgagccatgaagatccggaagtgaaatttaactggtatgtggatggcgtggaagtgcataacgcgaaaaccaaaccgcgcgaagaacagtataacagcacctatcgcgtggtgagcgtgctgaccgtgctgcatcaggattggctgaacggcaaagaatataaatgcaaagtgagcaacaaagcgctgccggcgccgattgaaaaaaccattagcaaagcgaaaggccagccgcgcgaaccgcaggtgtataccctgccgccgagccgcgatgaactgaccaaaaaccaggtgagcctgacctgcctggtgaaaggcttttatccgagcgatattgcggtggaatgggaaagcaacggccagccggaaaacaactataaaaccaccccgccggtgctggatagcgatggcagcttttttctgtatagcaaactgaccgtggataaaagccgctggcagcagggcaacgtgtttagctgcagcgtgatgcatgaagcgctgcataaccattatacccagaaaagcctgagcctgagcccgggc

SEQ ID NO: 28 - protein, 635 aa, Artificial sequence
  Homo sapiens
HGDGSFSDEMNTILDNLAARDFINWLIQTKITDGGGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 29 - protein, 655 aa, Artificial sequence
  Homo sapiens
METPAQLLFLLLLWLPDTTGHGDGSFSDEMNTILDNLAARDFINWLIQTKITDGGGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 30 - protein, 651 aa, Artificial sequence
  Homo sapiens
HGDGSFSDEMNTILDNLAARDFINWLIQTKITDHGDGSFSDEMNTILDNLAARDFINWLIQTKITDDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

SEQ ID NO: 31 - protein, 671 aa, Artificial sequence
  Homo sapiens
METPAQLLFLLLLWLPDTTGHGDGSFSDEMNTILDNLAARDFINWLIQTKITDHGDGSFSDEMNTILDNLAARDFINWLIQTKITDDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYKTTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASRAALGL

(19) United States
(12) Patent Application Publication
(10) Pub. No.: US 2020/0199192 A1
(43) Pub. Date: Jun. 25, 2020

(54) GLP-2 FUSION POLYPEPTIDES AND USES FOR TREATING AND PREVENTING GASTROINTESTINAL CONDITIONS

(71) Applicants: Kefeng SUN, Lexington, MA (US); Bettina STRACK-LOGUE, Lexington, MA (US); SHIRE-NPS PHARMACEUTICALS, INC., Lexington, MA (US)

(72) Inventors: Clark PAN, Lexington, MA (US); Angela NORTON, Lexington, MA (US); Bettina STRACK-LOGUE, Lexington, MA (US); Kefeng SUN, Lexington, MA (US)

(73) Assignee: SHIRE-NPS PHARMACEUTICALS, INC., Lexington, MA (US)

(21) Appl. No.: 16/640,965

(22) PCT Filed: Aug. 21, 2018
(86) PCT No.: PCT/US18/47171

§ 371 Date: Feb. 21, 2020

Related U.S. Application Data
(60) Provisional application No. 62/548,601, filed on Aug. 22, 2017.
(60) Provisional application No. 62/621,144, filed on Jan. 24, 2018.
(60) Provisional application No. 62/659,394, filed on Apr. 18, 2018.

(51) Int. Cl. C07K 14/605; A61K 47/68; A61P 1/00

(52) U.S. Cl. CPC C07K 14/605; A61K 38/00; A61P 1/00; A61K 47/68

(57) ABSTRACT

Described are fusion proteins of GLP-2 with an Fc region of immunoglobulin. The GLP-2 and Fc regions are separated by a linker comprised of amino acids. The fusion proteins persist and remain active in the body for longer periods of time than GLP-2 itself. Methods are disclosed of using the fusion proteins to treat and prevent enterocutaneous fistulae, radiation damage to the gastrointestinal tract, obstructive jaundice, and short bowel syndrome.

### CROSS-REFERENCE TO RELATED APPLICATIONS

This application claims priority to U.S. Provisional Application No. 62/548,601, filed on Aug. 22, 2017, U.S. Provisional Application No. 62/621,144, filed on Jan. 24, 2018, and U.S. Provisional Application No. 62/659,394, filed on Apr. 18, 2018, the disclosures of each of which is herein incorporated by reference in its entirety.

### TECHNICAL FIELD

Disclosed are mammalian GLP-2 fusion polypeptides and proteins and their use as therapeutics.

### BACKGROUND

Post-translational processing of proglucagon generates glucagon-like peptide-2 (GLP-2), a 33-amino acid intestinotrophic peptide hormone. GLP-2 acts to slow gastric emptying, reduce gastric secretions and increase intestinal blood flow. GLP-2 also stimulates growth of the large and small intestine at least by enhancing crypt cell proliferation and villus length so as to increase the surface area of the mucosal epithelium.

These effects suggest that GLP-2 can be used to treat a wide variety of gastrointestinal conditions. Demonstrated specific and beneficial effects of GLP-2 in the small intestine have raised much interest as to the use of GLP-2 in the treatment of intestinal disease or injury (Sinclair and Drucker, Physiology 2005: 357-65). Furthermore GLP-2 has been shown to prevent or reduce mucosal epithelial damage in a wide number of preclinical models of gut injury, including chemotherapy-induced mucositis, ischemia-reperfusion injury, dextran sulfate-induced colitis and genetic models of inflammatory bowel disease (Sinclair and Drucker, Physiology 2005:357-65).

However, administering GLP-2 by itself to human patients has not shown promise. GLP-2 has a short half-life that limits its use as a therapeutic because rapid in vivo cleavage of GLP-2 by dipeptidyl peptidase IV (DPP-IV) yields an essentially inactive peptide. Teduglutide, a GLP-2 therapeutic, has a substantially extended half-life due to substitution of alanine-2 with glycine. However, because teduglutide has a half-life of approximately 2 hours in healthy patients and 1.3 hours in SBS patients, daily dosing is needed.

Teduglutide has shown therapeutic promise in treating short bowel syndrome (SBS), which usually results from surgical resection of some or most of the small intestine for conditions such as Crohn's disease, mesenteric infarction, volvulus, trauma, congenital anomalies, and multiple strictures due to adhesions or radiation. Surgical resection may also include resection of all or part of the colon. SBS patients suffer from malabsorption of various nutrients (e.g., polypeptides, carbohydrates, fatty acids, vitamins, minerals, and water) that may lead to malnutrition, dehydration and weight loss. Some patients can maintain their protein and energy balance through hyperphagia, yet it is even rarer that patients can sustain fluid and electrolyte requirements to become independent from parenteral fluid.

GLP-2 may show promise in treating patients with enterocutaneous fistulae (ECF), a condition where gastric secretions bypass the small intestine via a fistula to the skin (Arebi, N. et al., Clin. Colon Rectal Surg., May 2004, 17(2):89-98). ECF can develop spontaneously from Crohn's disease and intra-abdominal cancer, or as a complication from Crohn's disease or radiotherapy. ECF has high morbidity and mortality at least because of infection, fluid loss, and malnutrition.

A DDP-IV resistant GLP-2 analogue showed promise in reducing radiation-induced apoptosis (Gu, J. et al., J. Controlled Release, 2017). Apoptosis occurs in radiation-induced small intestinal mucosal injury. In mice, GLP-2 also promoted CCD-18Co cell survival after radiation, protected against radiation-induced GI toxicity, down-regulated radiation-induced inflammatory responses, and decreased structural damage to the intestine after radiation.

GLP-2 may also show promise in treating patients with obstructive jaundice, a condition where intestinal barrier function is damaged (Chen, J. et al., World J. Gastroenterol., January 2015, 21(2):484-490). In rats, GLP-2 reduced the level of serum bilirubin and prevented structural damage to the intestinal mucosa.

There is a need to develop improved forms of GLP-2 to treat gastrointestinal conditions, including SBS, ECF, and pathology arising from radiation damage or obstructive jaundice. The improved forms remain active for a longer time period in the body such that less frequent dosing is needed.

### SUMMARY OF THE INVENTION

GLP-2 peptibodies are described herein. The peptibodies are generally fusion proteins between GLP-2 and either an Fc region or albumin. Pharmacokinetics data suggests that GLP-2 peptibodies may persist in the body longer than GLP-2 or even teduglutide or Gattex.

In one aspect is provided a glucagon-like peptide (GLP-2) peptibody selected from:
a) a GLP-2 peptibody comprising the amino acid sequence of

(SEQ ID NO: 1)
HGDGSFSDEMNTILDNLAARDFINWLIQTKITDGGGGGDKTHTCPPCPA
PEAAGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSV
MHEALHNHYTQKSLSLSPG,

b) a GLP-2 peptibody comprising the amino acid sequence of

(SEQ ID NO: 4)
HGDGSFSDEMNTILDNLAARDFINWLIQTKITDGGGGGDKTHTCPPCPA
PEAAGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSV
MHEALHNHYTQKSLSLSPGK,

c) a GLP-2 peptibody comprising the amino acid sequence of

(SEQ ID NO: 7)
HGDGSFSDEMNTILDNLAARDFINWLIQTKITDGGGGSGGGGSGGGGSD
KTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRW
QQGNVFSCSVMHEALHNHYTQKSLSLSPG,

d) a GLP-2 peptibody comprising the amino acid sequence of

(SEQ ID NO: 10)
HGDGSFSDEMNTILDNLAARDFINWLIQTKITDGGGGSGGGGSGGGGSD
KTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRW
QQGNVFSCSVMHEALHNHYTQKSLSLSPGK,

e) a GLP-2 peptibody comprising the amino acid sequence of

(SEQ ID NO: 13)
HGDGSFSDEMNTILDNLAARDFINWLIQTKITDDKTHTCPPCPAPEAAG
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEAL
HNHYTQKSLSLSPG,

f) a GLP-2 peptibody comprising the amino acid sequence of

(SEQ ID NO: 16)
HGDGSFSDEMNTILDNLAARDFINWLIQTKITDGGGGGGSGGGGSGGGG
SDKTHTCPPCPAPEAAGGPSVFLFPPKPKDTLMISRTPEVTCVVVDVSH
EDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKS
RWQQGNVFSCSVMHEALHNHYTQKSLSLSPG,

g) a GLP-2 peptibody comprising the amino acid sequence of

(SEQ ID NO: 19)
HGDGSFSDEMNTILDNLAARDFINWLIQTKITDGAPGGGGGAAAAAGGG
GGGAPGGGGGAAAAAGGGGGGAPGGGGGAAAAAGGGGGGAPDKTHTCPP
CPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFS
CSVMHEALHNHYTQKSLSLSPG,

h) a GLP-2 peptibody comprising the amino acid sequence of

(SEQ ID NO: 22)
HGDGSFSDEMNTILDNLAARDFINWLIQTKITDGGGGGGGDKTHTCPPC
PAPEAAGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSC
SVMHEALHNHYTQKSLSLSPG

or a pharmaceutically acceptable salt thereof,
i) a GLP-2 peptibody comprising the amino acid sequence of

(SEQ ID NO: 25)
HGDGSFSDEMNTILDNLAARDFINWLIQTKITDGGGGSGGGGSDKTHTC
PPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNV
FSCSVMHEALHNHYTQKSLSLSPG

j) a GLP-2 peptibody comprising the amino acid sequence of

(SEQ ID NO: 28)
HGDGSFSDEMNTILDNLAARDFINWLIQTKITDGGGGGGSGGGGSGGG
GSDAHKSEVAHRFKDLGEENFKALVLIAFAQYLQQCPFEDHVKLVNEVT
EFAKTCVADESAENCDKSLHTLFGDKLCTVATLRETYGEMADCCAKQEP
ERNECFLQHKDDNPNLPRLVRPEVDVMCTAFHDNEETFLKKYLYEIARR
HPYFYAPELLFFAKRYKAAFTECCQAADKAACLLPKLDELRDEGKASSA
KQRLKCASLQKFGERAFKAWAVARLSQRFPKAEFAEVSKLVTDLTKVHT
ECCHGDLLECADDRADLAKYICENQDSISSKLKECCEKPLLEKSHCIAE
VENDEMPADLPSLAADFVESKDVCKNYAEAKDVFLGMFLYEYARRHPDY
SVVLLLRLAKTYKTTLEKCCAAADPHECYAKVFDEFKPLVEEPQNLIKQ
NCELFEQLGEYKFQNALLVRYTKKVPQVSTPTLVEVSRNLGKVGSKCCK
HPEAKRMPCAEDYLSVVLNQLCVLHEKTPVSDRVTKCCTESLVNRRPCF
SALEVDETYVPKEFNAETFTFHADICTLSEKERQIKKQTALVELVKHKP
KATKEQLKAVMDDFAAFVEKCCKADDKETCFAEEGKKLVAASRAALGL,

and
k) a GLP-2 peptibody comprising the amino acid sequence of

(SEQ ID NO: 30)
HGDGSFSDEMNTILDNLAARDFINWLIQTKITDHGDGSFSDEMNTILDN
LAARDFINWLIQTKITDDAHKSEVAHRFKDLGEENFKALVLIAFAQYLQ
QCPFEDHVKLVNEVTEFAKTCVADESAENCDKSLHTLFGDKLCTVATLR
ETYGEMADCCAKQEPERNECFLQHKDDNPNLPRLVRPEVDVMCTAFHDN
EETFLKKYLYEIARRHPYFYAPELLFFAKRYKAAFTECCQAADKAACLL
PKLDELRDEGKASSAKQRLKCASLQKFGERAFKAWAVARLSQRFPKAEF
AEVSKLVTDLTKVHTECCHGDLLECADDRADLAKYICENQDSISSKLKE
CCEKPLLEKSHCIAEVENDEMPADLPSLAADFVESKDVCKNYAEAKDVF
LGMFLYEYARRHPDYSVVLLLRLAKTYKTTLEKCCAAADPHECYAKVFD
EFKPLVEEPQNLIKQNCELFEQLGEYKFQNALLVRYTKKVPQVSTPTLV
EVSRNLGKVGSKCCKHPEAKRMPCAEDYLSVVLNQLCVLHEKTPVSDRV
TKCCTESLVNRRPCFSALEVDETYVPKEFNAETFTFHADICTLSEKERQ
IKKQTALVELVKHKPKATKEQLKAVMDDFAAFVEKCCKADDKETCFAEE
GKKLVAASRAALGL;

or a pharmaceutically acceptable salt thereof.

In the aspect above, any of the sequences above (SEQ ID NOS: 1, 7, 13, 16, 19, 22 and 25) may further comprise a lysine (K) at the C-terminus.

In some embodiments, the GLP-2 peptibody is processed from a GLP-2 precursor polypeptide that comprises a signal peptide directly linked with GLP-2, with a linker between GLP-2 and an Fc region of any of IgG1, IgG2, IgG3 and IgG4. The signal peptide on the polypeptide may promote secretion of the GLP-2 peptibody from a mammalian host cell used to produce the GLP-2 peptibody, with the signal peptide cleaved from the GLP-2 peptibody after secretion. Any number of signal peptides may be used. The signal peptide may have the following sequence: METPAQLLFLLLWLPDTTG.

In some embodiments, the GLP-2 precursor polypeptide comprising a signal peptide is selected from:

a) a GLP-2 precursor polypeptide comprising the amino acid sequence of

(SEQ ID NO: 2)
METPAQLLFLLLLWLPDTTGHGDGSFSDEMNTILDNLAARDFINWLIQT
KITDGGGGGDKTHTCPPCPAPEAAGGPSVFLFPPKPKDTLMISRTPEVT
CVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLY
SKLTVDKSRWQQGNVFSCSVMHEALHNHYTQKSLSLSPG,

b) a GLP-2 precursor polypeptide comprising the amino acid sequence of

(SEQ ID NO: 5)
METPAQLLFLLLLWLPDTTGHGDGSFSDEMNTILDNLAARDFINWLIQT
KITDGGGGGDKTHTCPPCPAPEAAGGPSVFLFPPKPKDTLMISRTPEVT
CVVVDVSHEDPEVKFNWYVDGVEVHNAKTKPREEQYNSTYRVVSVLTVL
HQDWLNGKEYKCKVSNKALPAPIEKTISKAKGQPREPQVYTLPPSRDEL
TKNQVSLTCLVKGFYPSDIAVEWESNGQPENNYKTTPPVLDSDGSFFLY
SKLTVDKSRWQQGNVFSCSVMHEALHNHYTQKSLSLSPGK,

c) a GLP-2 precursor polypeptide comprising the amino acid sequence of

(SEQ ID NO: 8)
METPAQLLFLLLLWLPDTTGHGDGSFSDEMNTILDNLAARDFINWLIQT
KITDGGGGSGGGGSGGGGSDKTHTCPPCPAPEAAGGPSVFLFPPKPKDT
LMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSP
G,

d) a GLP-2 precursor polypeptide comprising the amino acid sequence of

(SEQ ID NO: 11)
METPAQLLFLLLLWLPDTTGHGDGSFSDEMNTILDNLAARDFINWLIQT
KITDGGGGSGGGGSGGGGSDKTHTCPPCPAPEAAGGPSVFLFPPKPKDT
LMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
K,

e) a GLP-2 precursor polypeptide comprising the amino acid sequence of

(SEQ ID NO: 14)
METPAQLLFLLLLWLPDTTGHGDGSFSDEMNTILDNLAARDFINWLIQT
KITDDKTHTCPPCPAPEAAGGPSVFLFPPKPKDTLMISRTPEVTCVVVD
VSHEDPEVKFNWYVDGVEVHNAKTKPREEQYNSTYRVVSVLTVLHQDWL
NGKEYKCKVSNKALPAPIEKTISKAKGQPREPQVYTLPPSRDELTKNQV
SLTCLVKGFYPSDIAVEWESNGQPENNYKTTPPVLDSDGSFFLYSKLTV
DKSRWQQGNVFSCSVMHEALHNHYTQKSLSLSPG,

f) a GLP-2 precursor polypeptide comprising the amino acid sequence of

(SEQ ID NO: 17)
METPAQLLFLLLLWLPDTTGHGDGSFSDEMNTILDNLAARDFINWLIQT
KITDGGGGGGSGGGGSGGGGSDKTHTCPPCPAPEAAGGPSVFLFPPKPK
DTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLS
PG,

g) a GLP-2 precursor polypeptide comprising the amino acid sequence of

(SEQ ID NO: 20)
METPAQLLFLLLLWLPDTTGHGDGSFSDEMNTILDNLAARDFINWLIQT
KITDGAPGGGGGAAAAAGGGGGGAPGGGGGAAAAAGGGGGGAPGGGGGA
AAAAGGGGGGAPDKTHTCPPCPAPEAAGGPSVFLFPPKPKDTLMISRTP
EVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSF
FLYSKLTVDKSRWQQGNVFSCSVMHEALHNHYTQKSLSLSPG,

h) a GLP-2 precursor polypeptide comprising the amino acid sequence of

(SEQ ID NO: 23)
METPAQLLFLLLLWLPDTTGHGDGSFSDEMNTILDNLAARDFINWLIQT
KITDGGGGGGGDKTHTCPPCPAPEAAGGPSVFLFPPKPKDTLMISRTPE
VTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFF
LYSKLTVDKSRWQQGNVFSCSVMHEALHNHYTQKSLSLSPG

or a pharmaceutically acceptable salt thereof,
i) a GLP-2 precursor polypeptide comprising the amino acid sequence of

(SEQ ID NO: 26)
METPAQLLFLLLLWLPDTTGHGDGSFSDEMNTILDNLAARDFINWLIQT
KITDGGGGSGGGGSDKTHTCPPCPAPEAAGGPSVFLFPPKPKDTLMISR
TPEVTCVVVDVSHEDPEVKFNWYVDGVEVHNAKTKPREEQYNSTYRVVS
VLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDG
SFFLYSKLTVDKSRWQQGNVFSCSVMHEALHNHYTQKSLSLSPG

j) a GLP-2 precursor polypeptide comprising the amino acid sequence of

(SEQ ID NO: 29)
HGDGSFSDEMNTILDNLAARDFINWLIQTKITDGGGGGGSGGGGSGGGG
SDAHKSEVAHRFKDLGEENFKALVLIAFAQYLQQCPFEDHVKLVNEVTE
FAKTCVADESAENCDKSLHTLFGDKLCTVATLRETYGEMADCCAKQEPE
RNECFLQHKDDNPNLPRLVRPEVDVMCTAFHDNEETFLKKYLYEIARRH
PYFYAPELLFFAKRYKAAFTECCQAADKAACLLPKLDELRDEGKASSAK
QRLKCASLQKFGERAFKAWAVARLSQRFPKAEFAEVSKLVTDLTKVHTE
CCHGDLLECADDRADLAKYICENQDSISSKLKECCEKPLLEKSHCIAEV
ENDEMPADLPSLAADFVESKDVCKNYAEAKDVFLGMFLYEYARRHPDYS
VVLLLRLAKTYKTTLEKCCAAADPHECYAKVFDEFKPLVEEPQNLIKQN
CELFEQLGEYKFQNALLVRYTKKVPQVSTPTLVEVSRNLGKVGSKCCKH
PEAKRMPCAEDYLSVVLNQLCVLHEKTPVSDRVTKCCTESLVNRRPCFS
ALEVDETYVPKEFNAETFTFHADICTLSEKERQIKKQTALVELVKHKPK
ATKEQLKAVMDDFAAFVEKCCKADDKETCFAEEGKKLVAASRAALGL,

and
k) a GLP-2 precursor polypeptide comprising the amino acid sequence of

(SEQ ID NO: 30)
HGDGSFSDEMNTILDNLAARDFINWLIQTKITDHGDGSFSDEMNTILDN
LAARDFINWLIQTKITDDAHKSEVAHRFKDLGEENFKALVLIAFAQYLQ
QCPFEDHVKLVNEVTEFAKTCVADESAENCDKSLHTLFGDKLCTVATLR
ETYGEMADCCAKQEPERNECFLQHKDDNPNLPRLVRPEVDVMCTAFHDN
EETFLKKYLYEIARRHPYFYAPELLFFAKRYKAAFTECCQAADKAACLL
PKLDELRDEGKASSAKQRLKCASLQKFGERAFKAWAVARLSQRFPKAEF
AEVSKLVTDLTKVHTECCHGDLLECADDRADLAKYICENQDSISSKLKE
CCEKPLLEKSHCIAEVENDEMPADLPSLAADFVESKDVCKNYAEAKDVF
LGMFLYEYARRHPDYSVVLLLRLAKTYKTTLEKCCAAADPHECYAKVFD
EFKPLVEEPQNLIKQNCELFEQLGEYKFQNALLVRYTKKVPQVSTPTLV
EVSRNLGKVGSKCCKHPEAKRMPCAEDYLSVVLNQLCVLHEKTPVSDRV
TKCCTESLVNRRPCFSALEVDETYVPKEFNAETFTFHADICTLSEKERQ
IKKQTALVELVKHKPKATKEQLKAVMDDFAAFVEKCCKADDKETCFAEE
GKKLVAASRAALGL;

or a pharmaceutically acceptable salt thereof.

Any of the GLP-2 precursor polypeptide sequences above (SEQ ID NOS: 2, 8, 14, 17, 20, 23 and 26) may further comprise a lysine (K) at the C-terminus.

The Fc region may be IgG1 with the LALA mutation. The GLP-2 precursor polypeptide comprising a signal peptide can have the following formula:

Signal Peptide-GLP-2[A2G]-linker-IgG1(LALA)

In some embodiments, the pharmaceutical compositions described herein further comprise a carrier or a pharmaceutically acceptable excipient. In some embodiments, the pharmaceutical compositions are formulated as a liquid suitable for administration by injection or infusion. In some embodiments, the pharmaceutical compositions are formulated for sustained release, extended release, delayed release or slow release of the GLP-2 peptibody, e.g., GLP-2 peptibody comprising SEQ ID NO: 1 or GLP-2 peptibody comprising the amino acid sequence of SEQ ID NO: 7. In some embodiments, the GLP-2 peptibody, e.g., GLP-2 peptibody comprising the amino acid sequence of SEQ ID NO: 1 or 7, is administered in a concentration of 10 to 200 mg/mL. In some embodiments, the GLP-2 peptibody comprises the amino acid sequence of SEQ ID NO: 28 or SEQ ID NO: 30, and is administered in a concentration of 10 to 1000 mg/mL or 50 to 500 mg/mL.

In another aspect is provided a polynucleotide comprising a sequence encoding the GLP-2 peptibodies described herein. The sequence may be that set forth in SEQ ID NOS: 3, 9, 15, 18, 21, 24 or 27. In some embodiments, the polynucleotide comprises a sequence encoding a GLP-2 peptibody comprising the amino acid sequence of SEQ ID NO: 1. In some embodiments, the polynucleotide comprises the sequence of SEQ ID NO: 3. In some embodiments, the polynucleotide comprises a sequence encoding a GLP-2 peptibody comprising the amino acid sequence of SEQ ID NO: 7. In some embodiments, the polynucleotide comprises the sequence of SEQ ID NO: 9. In some embodiments, a vector is provided comprising any of the polynucleotides disclosed herein. In the vector, a polynucleotide may be operably linked to a promoter.

In another aspect is provided a host cell comprising the polynucleotide. In some embodiments, the host cell is a Chinese hamster ovary cell. In some embodiments, the host cell expresses GLP-2 peptibody at levels sufficient for fed-batch cell culture scale.

In another aspect is provided a method for treating a patient with enterocutaneous fistula (ECF) comprising treating the patient with a GLP-2 peptibody, e.g., a GLP-2 peptibody comprising SEQ ID NO: 1 or SEQ ID NO: 7, using a dosing regimen effective to promote closure, healing, and/or repair of the ECF. The GLP-2 peptibody, e.g., GLP-2 peptibody comprising SEQ ID NO: 1 or SEQ ID NO: 7, may be administered subcutaneously or intravenously. In some embodiments, the GLP-2 peptibody comprises the amino acid sequence of SEQ ID NO: 1. In some embodiments, the GLP-2 peptibody comprises the amino acid sequence of SEQ ID NO: 7. In some embodiments, the method is effective to enhance intestinal absorption by said patient. In some embodiments, the method is effective to enhance intestinal absorption of nutrients, e.g., polypeptides, carbohydrates, fatty acids, vitamins, minerals, and water. In some embodiments, the method is effective to reduce the volume of gastric secretions in said patient. In some embodiments, the method is effective to increase villus height in small intestine of said patient. In some embodiments, the method is effective to increase crypt depth in small intestine of said patient.

In some embodiments, the GLP-2 peptibody is administered subcutaneously. In some embodiments, the GLP-2 peptibody is administered subcutaneously according to a dosage regimen of between 0.02 to 3.0 mg/kg once every 2-14 days. In some embodiments, the GLP-2 peptibody is administered subcutaneously according to a dosage regimen of between 0.2 to 1.4 mg/kg once every 7-14 days. In some embodiments, the GLP-2 peptibody is administered subcutaneously according to a dosage regimen of between 0.3 to 1.0 mg/kg once every week. In some embodiments, the administered GLP-2 peptibody is in a concentration of 10 to 200 mg/mL. In some embodiments, the GLP-2 peptibody comprises the amino acid sequence of SEQ ID NOS: 1 or 7 and the GLP-2 peptibody is administered subcutaneously according to a dosage regimen of between 0.02 to 0.5 mg/kg once every 2-14 days. In some embodiments, the GLP-2 peptibody comprises the amino acid sequence of SEQ ID NOS: 1 or 7 and the GLP-2 peptibody is in a concentration of 10 to 200 mg/mL. Alternatively, the GLP-2 peptibody could be administered every three weeks or once a month, such as for maintenance purposes.

In some embodiments, the GLP-2 peptibody is administered intravenously. In some embodiments, the GLP-2 peptibody is administered intravenously according to a dosage regimen of between 0.02 to 3.0 mg/kg once every 2-14 days. In some embodiments, the GLP-2 peptibody is administered subcutaneously according to a dosage regimen of between 0.2 to 1.4 mg/kg once every 7-14 days. In some embodiments, the GLP-2 peptibody is administered subcutaneously according to a dosage regimen of between 0.3 to 1.0 mg/kg once every week. In some embodiments, the administered GLP-2 peptibody is in a concentration of 10 to 200 mg/mL.

In some embodiments, the GLP-2 peptibody comprises the amino acid sequence of SEQ ID NOS: 1 or 7 and the GLP-2 peptibody is administered intravenously according to a dosage regimen of between 0.02 to 3.0 once every 2-14 days. In some embodiments, the GLP-2 peptibody comprises the amino acid sequence of SEQ ID NO: 7 and the GLP-2 peptibody is administered intravenously according to a dosage regimen of between 0.2 to 1.4 mg/kg once every 7-14 days.

In some embodiments, the GLP-2 peptibody is administered intravenously according to a dosage regimen of between 0.3 to 1.0 mg/kg once every week. In some embodiments, the GLP-2 peptibody comprises the amino acid sequence of SEQ ID NOS: 1 or 7 and the GLP-2 peptibody is in a concentration of 10 to 200 mg/mL.

In another aspect is provided a method for treating a patient with obstructive jaundice comprising treating the patient with a GLP-2 peptibody, e.g., GLP-2 peptibody comprising SEQ ID NO: 1 or SEQ ID NO: 7, using a dosing regimen effective to treat the obstructive jaundice. In some embodiments, the GLP-2 peptibody comprises the amino acid sequence of SEQ ID NO: 1. In some embodiments, the GLP-2 peptibody comprises the amino acid sequence of SEQ ID NO: 7. In some embodiments, the level of serum bilirubin is reduced as compared to the level of serum bilirubin before said treatment. In some embodiments, the level of serum bilirubin is reduced as compared to the level of serum bilirubin before said treatment. In some embodiments, the method is effective to enhance intestinal absorption by said patient. In some embodiments, the method is effective to enhance intestinal absorption of nutrients, e.g., polypeptides, carbohydrates, fatty acids, vitamins, minerals, and water. In some embodiments, the method is effective to reduce the volume of gastric secretions in said patient. In some embodiments, the method is effective to increase villus height in the small intestine of said patient. In some embodiments, the method is effective to increase crypt depth in the small intestine of said patient. In some embodiments, the method is effective to increase crypt organization in the small intestine of said patient. In some embodiments, the method is effective to improve intestinal barrier function in said patient and to reduce the rate of bacteria translocation across the small intestine of said patient.

In some embodiments, the GLP-2 peptibody is administered subcutaneously. In some embodiments, the GLP-2 peptibody is administered subcutaneously according to a dosage regimen of between 0.02 to 3.0 mg/kg once every 2-14 days. In some embodiments, the GLP-2 peptibody is administered subcutaneously according to a dosage regimen of between 0.2 to 1.4 mg/kg once every 7-14 days. In some embodiments, the GLP-2 peptibody is administered subcutaneously according to a dosage regimen of between 0.3 to 1.0 mg/kg once every week. In some embodiments, the administered GLP-2 peptibody is in a concentration of 10 to 200 mg/mL.

In some embodiments, the GLP-2 peptibody comprises the amino acid sequence of SEQ ID NOS: 1 or 7 and the GLP-2 peptibody is administered subcutaneously according to a dosage regimen of between 0.02 to 3.0 mg/kg once every 2-14 days. In some embodiments, the GLP-2 peptibody comprises the amino acid sequence of SEQ ID NO: 7 and the GLP-2 peptibody is administered subcutaneously according to a dosage regimen of between 0.2 to 1.4 mg/kg once every 7-14 days. In some embodiments, the GLP-2 peptibody is administered intravenously according to a dosage regimen of between 0.3 to 1.0 mg/kg once every week. In some embodiments, the GLP-2 peptibody comprises the amino acid sequence of SEQ ID NOS: 1 or 7 and the GLP-2 peptibody is in a concentration of 10 to 200 mg/mL.

In some embodiments, the GLP-2 peptibody is administered intravenously. In some embodiments, the GLP-2 peptibody is administered intravenously according to a dosage regimen of between 0.02 to 3.0 once every 2-14 days. In some embodiments, the GLP-2 peptibody is administered intravenously according to a dosage regimen of between 0.2 to 1.4 mg/kg once every 7-14 days. In some embodiments, the GLP-2 peptibody is administered intravenously according to a dosage regimen of between 0.3 to 1.0 mg/kg once every week. In some embodiments, the administered GLP-2 peptibody is in a concentration of 10 to 200 mg/mL.

In some embodiments, the GLP-2 peptibody comprises the amino acid sequence of SEQ ID NOS: 1 or 7 and the GLP-2 peptibody is administered intravenously according to a dosage regimen of between 0.02 to 3.0 once every 2-14 days. In some embodiments, the GLP-2 peptibody comprises the amino acid sequence of SEQ ID NO: 7 and the GLP-2 peptibody is administered intravenously according to a dosage regimen of between 0.2 to 1.4 mg/kg once every 7-14 days. In some embodiments, the GLP-2 peptibody comprises the amino acid sequence of SEQ ID NO: 7 and is administered intravenously according to a dosage regimen of between 0.3 to 1.0 mg/kg once every week. In some embodiments, the GLP-2 peptibody comprises the amino acid sequence of SEQ ID NOS: 1 or 7 and the GLP-2 peptibody is in a concentration of 10 to 200 mg/mL.

In another aspect, the present invention provides a method for treating, ameliorating or protecting against radiation damage, and/or the effects thereof, to the gastrointestinal tract, comprising administering a GLP-2 peptibody, e.g., GLP-2 peptibody comprising SEQ ID NO: 1 or SEQ ID NO: 7. The dosing regimen is effective to treat or prevent radiation damage to the gastrointestinal tract of the patient. In some embodiments, the GLP-2 peptibody comprises the amino acid sequence of SEQ ID NO: 1. In some embodiments, the GLP-2 peptibody comprises the amino acid sequence of SEQ ID NO: 7. In some embodiments, the radiation damage is in the small intestine. In some embodiments, the method is effective to reduce apoptosis in cells of the gastrointestinal tract. In some embodiments, the GLP-2 peptibody may be administered before, while, or after the patient is treated with radiation or radiotherapy.

In some embodiments, the method is effective to reduce apoptosis in cells of the gastrointestinal tract. In some embodiments, the method is effective to increase villus height in the small intestine of said patient. In some embodiments, the method is effective to increase crypt depth in the small intestine of said patient. In some embodiments, the method is effective to increase crypt organization in the small intestine of said patient. In some embodiments, the method is effective to improve intestinal barrier function in said patient.

In some embodiments, the GLP-2 peptibody is administered subcutaneously. In some embodiments, the GLP-2 peptibody is administered subcutaneously according to a dosage regimen of between 0.02 to 3.0 mg/kg once every 2-14 days. In some embodiments, the GLP-2 peptibody is administered subcutaneously according to a dosage regimen of between 0.2 to 1.4 mg/kg once every 7-14 days. In some embodiments, the GLP-2 peptibody is administered subcutaneously according to a dosage regimen of between 0.3 to 1.0 mg/kg once every week. In some embodiments, the administered GLP-2 peptibody is in a concentration of 10 to 200 mg/mL.

In some embodiments, the GLP-2 peptibody comprises the amino acid sequence of SEQ ID NOS: 1 or 7 and the GLP-2 peptibody is administered subcutaneously according to a dosage regimen of between 0.02 to 3.0 mg/kg once every 2-14 days. In some embodiments, the GLP-2 peptibody comprises the amino acid sequence of SEQ ID NO: 7 and the GLP-2 peptibody is administered subcutaneously according to a dosage regimen of between 0.2 to 1.4 mg/kg once every 7-14 days. In some embodiments, the GLP-2 peptibody is administered subcutaneously according to a dosage regimen of between 0.3 to 1.0 mg/kg once every week. In some embodiments, the GLP-2 peptibody comprises the amino acid sequence of SEQ ID NOS: 1 or 7 and the GLP-2 peptibody is in a concentration of 10 to 200 mg/mL.

In some embodiments, the GLP-2 peptibody is administered intravenously. In some embodiments, the GLP-2 peptibody is administered intravenously according to a dosage regimen of between 0.02 to 3.0 mg/kg, 0.2 to 1.4 mg/kg, or 0.3 to 1.0 mg/kg once every 2-14 days. In some embodiments, the administered GLP-2 peptibody is in a concentration of 10 to 200 mg/mL. In some embodiments, the GLP-2 peptibody comprises the amino acid sequence of SEQ ID NOS: 1 or 7 and the GLP-2 peptibody is administered intravenously according to a dosage regimen of between 0.02 to 3.0 mg/kg once every 2-14 days. In some embodiments, the GLP-2 peptibody comprises the amino acid sequence of SEQ ID NO: 7 and the GLP-2 peptibody is administered intravenously according to a dosage regimen of between 0.2 to 1.4 mg/kg once every 7-14 days. In some embodiments, the GLP-2 peptibody is administered intravenously according to a dosage regimen of between 0.3 to 1.0 mg/kg once every week. In some embodiments, the GLP-2 peptibody comprises the amino acid sequence of SEQ ID NOS: 1 or 7 and the GLP-2 peptibody is in a concentration of 10 to 200 mg/mL.

In another aspect, the present invention provides a method for treating, ameliorating or preventing radiation-induced enteritis, and/or the effects thereof, to the gastrointestinal tract, comprising administering a GLP-2 peptibody, e.g., GLP-2 peptibody comprising SEQ ID NO: 1 or SEQ ID NO: 7. In some embodiments, the GLP-2 peptibody comprises the amino acid sequence of SEQ ID NO: 1. In some embodiments, the GLP-2 peptibody comprises the amino acid sequence of SEQ ID NO: 7. In some embodiments, the method is effective to reduce apoptosis in cells of the gastrointestinal tract. In some embodiments, the method is effective to increase villus height in the small intestine of said patient. In some embodiments, the method is effective to increase crypt depth in the small intestine of said patient. In some embodiments, the method is effective to increase crypt organization in the small intestine of said patient. In some embodiments, the method is effective to improve intestinal barrier function in said patient.

In some embodiments, the GLP-2 peptibody is administered subcutaneously. In some embodiments, the GLP-2 peptibody is administered subcutaneously according to a dosage regimen of between 0.02 to 3.0 mg/kg once every 2-14 days. In some embodiments, the GLP-2 peptibody is administered subcutaneously according to a dosage regimen of between 0.2 to 1.4 mg/kg once every 7-14 days. In some embodiments, the GLP-2 peptibody is administered subcutaneously according to a dosage regimen of between 0.3 to 1.0 mg/kg once every week. In some embodiments, the administered GLP-2 peptibody is in a concentration of 10 to 200 mg/mL.

In some embodiments, the GLP-2 peptibody comprises the amino acid sequence of SEQ ID NOS: 1 or 7 and the GLP-2 peptibody is administered subcutaneously according to a dosage regimen of between 0.02 to 3.0 mg/kg once every 2-14 days. In some embodiments, the GLP-2 peptibody comprises the amino acid sequence of SEQ ID NO: 7 and the GLP-2 peptibody is administered subcutaneously according to a dosage regimen of between 0.2 to 1.4 mg/kg once every 7-14 days, or of between 0.3 to 1.0 mg/kg once every week. In some embodiments, the GLP-2 peptibody comprises the amino acid sequence of SEQ ID NOS: 1 or 7 and the GLP-2 peptibody is in a concentration of 10 to 200 mg/mL.

In some embodiments, the GLP-2 peptibody is administered intravenously. In some embodiments, the GLP-2 peptibody is administered intravenously according to a dosage regimen of between 0.02 to 3.0 mg/kg once every 2-14 days. In some embodiments, the GLP-2 peptibody is administered intravenously according to a dosage regimen of between 0.2 to 1.4 mg/kg once every 7-14 days. In some embodiments, the GLP-2 peptibody is administered intravenously according to a dosage regimen of between 0.3 to 1.0 mg/kg once every week. In some embodiments, the administered GLP-2 peptibody is in a concentration of 10 to 200 mg/mL.

In some embodiments, the GLP-2 peptibody comprises the amino acid sequence of SEQ ID NOS: 1 or 7 and the GLP-2 peptibody is administered intravenously according to a dosage regimen of between 0.02 to 3.0 mg/kg once every 2-14 days. In some embodiments, the GLP-2 peptibody comprises the amino acid sequence of SEQ ID NO: 7 and the GLP-2 peptibody is administered intravenously according to a dosage regimen of between 0.2 to 1.4 mg/kg once every 7-14 days, or of between 0.3 to 1.0 mg/kg once every week. In some embodiments, the GLP-2 peptibody comprises the amino acid sequence of SEQ ID NOS: 1 or 7 and the GLP-2 peptibody is in a concentration of 10 to 200 mg/mL.

In another aspect is provided a method for treating a patient with short bowel syndrome presenting with colon in continuity with remnant small intestine comprising treating the patient with GLP-2 peptibody, e.g., the GLP-2 peptibody comprising SEQ ID NO: 1 or SEQ ID NO: 7, using a dosing regimen effective to treat the short bowel syndrome. In some embodiments, the GLP-2 peptibody comprises the amino acid sequence of SEQ ID NO: 1. In some embodiments, the GLP-2 peptibody comprises the amino acid sequence of SEQ ID NO: 7. In some embodiments, the remnant small intestine has a length of at least 25 cm. In some embodiments, the remnant small intestine has a length of at least 50 cm. In some embodiments, the remnant small intestine has a length of at least 75 cm. In some embodiments, the GLP-2 peptibody is administered as a medicament for enhancing intestinal absorption in short bowel syndrome patients presenting with at least about 25% colon-in-continuity with remnant small intestine.

In some embodiments, the method is effective to enhance intestinal absorption in said patient. In some embodiments, the method is effective to enhance intestinal absorption of nutrients, e.g., polypeptides, amino acids, carbohydrates, fatty acids, vitamins, minerals, and water. In some embodiments, the method is effective to increase villus height in the small intestine of said patient. In some embodiments, the method is effective to increase crypt depth in the small intestine of said patient. In some embodiments, the method is effective to increase crypt organization in the small intestine of said patient. In some embodiments, the method is effective to improve intestinal barrier function in said patient. In some embodiments, the method is effective to decrease fecal wet weight, increase urine wet weight, increase energy absorption across the small intestine, and/or increase water absorption across the small intestine. The energy absorption can include increased absorption of one or more of polypeptides, amino acids, carbohydrates and fatty acids. In some embodiments, the patient is dependent on parenteral nutrition.

In some embodiments, the GLP-2 peptibody is administered subcutaneously. In some embodiments, the GLP-2 peptibody is administered subcutaneously according to a dosage regimen of between 0.02 to 3.0 mg/kg once every 2-14 days. In some embodiments, the GLP-2 peptibody is administered subcutaneously according to a dosage regimen of between 0.2 to 1.4 mg/kg once every 7-14 days. In some embodiments, the GLP-2 peptibody is administered subcutaneously according to a dosage regimen of between 0.3 to 1.0 mg/kg once every week. In some embodiments, the administered GLP-2 peptibody is in a concentration of 10 to 200 mg/mL.

In some embodiments, the GLP-2 peptibody comprises the amino acid sequence of SEQ ID NOS: 1 or 7 and the GLP-2 peptibody is administered subcutaneously according to a dosage regimen of between 0.02 to 3.0 mg/kg once every 2-14 days. In some embodiments, the GLP-2 peptibody comprises the amino acid sequence of SEQ ID NO: 7 and the GLP-2 peptibody is administered subcutaneously according to a dosage regimen of between 0.2 to 1.4 mg/kg once every 7-14 days, or of between 0.3 to 1.0 mg/kg once every week. In some embodiments, the GLP-2 peptibody comprises the amino acid sequence of SEQ ID NOS: 1 or 7 and the GLP-2 peptibody is in a concentration of 10 to 200 mg/mL.

In some embodiments, the GLP-2 peptibody is administered intravenously. In some embodiments, the GLP-2 peptibody is administered intravenously according to a dosage regimen of between 0.02 to 3.0 mg/kg once every 2-14 days. In some embodiments, the GLP-2 peptibody is administered intravenously according to a dosage regimen of between 0.2 to 1.4 mg/kg once every 7-14 days. In some embodiments, the GLP-2 peptibody is administered intravenously according to a dosage regimen of between 0.3 to 1.0 mg/kg once every week. In some embodiments, the administered GLP-2 peptibody is in a concentration of 10 to 200 mg/mL.

In some embodiments, the GLP-2 peptibody comprises the amino acid sequence of SEQ ID NOS: 1 or 7 and the GLP-2 peptibody is administered intravenously according to a dosage regimen of between 0.02 to 3.0 mg/kg once every 2-14 days. In some embodiments, the GLP-2 peptibody comprises the amino acid sequence of SEQ ID NO: 7 and the GLP-2 peptibody is administered intravenously according to a dosage regimen of between 0.2 to 1.4 mg/kg once every 7-14 days, or of between 0.3 to 1.0 mg/kg once every week. In some embodiments, the GLP-2 peptibody comprises the amino acid sequence of SEQ ID NOS: 1 or 7 and the GLP-2 peptibody is in a concentration of 10 to 200 mg/mL.

In any of the aspects and embodiments described herein, the GLP-2 peptibody, e.g., GLP-2 peptibody comprising SEQ ID NO: 1 or SEQ ID NO: 7, may be administered subcutaneously or intravenously. The GLP-2 peptibody comprising SEQ ID NO: 1 or SEQ ID NO: 7 may be administered subcutaneously according to a dosage regimen of between 0.02 to 3.0 mg/kg, 0.02 to 0.5 mg/kg, 0.04 to 0.45 mg/kg, 0.08 to 0.4 mg/kg, 0.10 to 0.35 mg/kg, 0.20 to 0.30 mg/kg, 0.02 to 0.05 mg/kg, 0.03 to 0.04 mg/kg, 0.05 to 0.10 mg/kg, 0.10 to 0.15 mg/kg, 0.2 to 0.3 mg/kg, 0.3 to 0.4 mg/kg, 0.4 to 0.5 mg/kg, 0.5 to 0.8 mg/kg, 0.7 to 1.0 mg/kg, 0.9 to 1.2 mg/kg, 1.0 to 1.5 mg/kg, 1.2 to 1.8 mg/kg, 1.5 to 2.0 mg/kg, 1.7 to 2.5 mg/kg, or 2.0 to 3.0 mg/kg, once every 2-14 days, every 5-8 days, or every week (QW). The GLP-2 peptibody (e.g., comprising the amino acid sequence of SEQ ID NO: 7) may be administered subcutaneously according to a dosage regimen of between 0.2 to 1.4 mg/kg, 0.3 to 1.0 mg/kg, 0.4 to 0.9 mg/kg, 0.5 to 0.8 mg/kg, 0.3 to 0.7 mg/kg, 0.6 to 1.0 mg/kg, 0.2 to 0.4 mg/kg, 0.3 to 0.5 mg/kg, 0.4 to 0.6 mg/kg, 0.5 to 0.7 mg/kg, 0.6 to 0.8 mg/kg, 0.7 to 0.9 mg/kg, 0.8 to 1.0 mg/kg, 0.9 to 1.1 mg/kg, 1.0 to 1.2 mg/kg, 1.1 to 1.3 mg/kg, and 1.2 to 1.4 mg/kg, every week (QW) or every two weeks.

Alternatively, the GLP-2 peptibody could be administered according to a dosage regimen of between 0.2 to 1.4 mg/kg, 0.3 to 1.0 mg/kg, 0.4 to 0.9 mg/kg, 0.5 to 0.8 mg/kg, 0.3 to 0.7 mg/kg, 0.6 to 1.0 mg/kg, 0.2 to 0.4 mg/kg, 0.3 to 0.5 mg/kg, 0.4 to 0.6 mg/kg, 0.5 to 0.7 mg/kg, 0.6 to 0.8 mg/kg, 0.7 to 0.9 mg/kg, 0.8 to 1.0 mg/kg, 0.9 to 1.1 mg/kg, 1.0 to 1.2 mg/kg, 1.1 to 1.3 mg/kg, and 1.2 to 1.4 mg/kg every three weeks or once a month, such as for maintenance purposes. The GLP-2 peptibody (e.g., comprising the amino acid sequence of SEQ ID NO: 1 or SEQ ID NO: 7) may be administered subcutaneously according to a dosage regimen of between 0.02 to 0.5 mg/kg, 0.04 to 0.45 mg/kg, 0.08 to 0.4 mg/kg, 0.10 to 0.35 mg/kg, 0.20 to 0.30 mg/kg every 5-8 days, or every week (QW), such as for maintenance purposes. The GLP-2 peptibody comprising SEQ ID NO: 1 or SEQ ID NO: 7 may be administered in a concentration of 10 to 200 mg/mL, 10 to 180 mg/mL, 20 to 160 mg/mL, 25 to 150 mg/mL, 30 to 125 mg/mL, 50 to 100 mg/mL, 60 to 90 mg/mL, about 75 mg/mL, 75 mg/mL, 10 to 20 mg/mL, 15 to 25 mg/mL, 12 to 18 mg/mL, 13-17 mg/mL, 14-16 mg/mL, about 15 mg/mL or 15 mg/mL.

### BRIEF DESCRIPTION OF THE DRAWINGS

FIG. 1A shows the amino acid sequence of SEQ ID NO: 1. The GLP-2[A2G] sequence is underlined and the linker is bolded. A linker sequence and the IgG1 Fc sequence follows the GLP-2 sequence. The GLP-2 peptibody B264 has the amino acid sequence set forth in SEQ ID NO: 1.

FIG. 1B shows the amino acid sequence of SEQ ID NO: 2, which has a signal peptide sequence fused to the N-terminus of the amino acid sequence of SEQ ID NO: 1.

FIG. 1C shows a nucleotide sequence of SEQ ID NO: 3 that encodes the GLP-2 peptibody of SEQ ID NO: 2.

FIG. 1D shows both the nucleotide sequence of SEQ ID NO: 3 and the amino acid sequence of SEQ ID NO: 2.

FIG. 1E shows the amino acid sequence of SEQ ID NO: 4. The GLP-2[A2G] sequence is underlined and the linker is bolded. A linker sequence and the IgG1 Fc sequence follows the GLP-2 sequence. The GLP-2 peptibody B has the amino acid sequence set forth in SEQ ID NO: 4.

FIG. 1F shows the amino acid sequence of SEQ ID NO: 5, which has a signal peptide sequence fused to the N-terminus of the amino acid sequence of SEQ ID NO: 4.

FIG. 1G shows a nucleotide sequence of SEQ ID NO: 6 that encodes the GLP-2 peptibody of SEQ ID NO: 5.

FIG. 1H shows both the nucleotide sequence of SEQ ID NO: 6 and the amino acid sequence of SEQ ID NO: 5.

FIG. 2A shows the amino acid sequence of SEQ ID NO: 7. The GLP-2[A2G] sequence is underlined and the linker is bolded. A linker sequence and the IgG1 Fc sequence follows the GLP-2 sequence. The GLP-2 peptibody K274 has the amino acid sequence set forth in SEQ ID NO: 7.

FIG. 2B shows the amino acid sequence of SEQ ID NO: 8, which has a signal peptide sequence fused to the N-terminus of the amino acid sequence of SEQ ID NO: 7.

FIG. 2C shows a nucleotide sequence of SEQ ID NO: 9 that encodes the GLP-2 peptibody of SEQ ID NO: 8.

FIG. 2D shows both the nucleotide sequence of SEQ ID NO: 9 and the amino acid sequence of SEQ ID NO: 8.

FIG. 2E shows the amino acid sequence of SEQ ID NO: 10. The GLP-2 sequence is underlined and the linker is bolded. A linker sequence and the IgG1 Fc sequence follows the GLP-2 sequence. The GLP-2 peptibody K has the amino acid sequence set forth in SEQ ID NO: 10.

FIG. 2F shows the amino acid sequence of SEQ ID NO: 11, which has a signal peptide sequence fused to the N-terminus of the amino acid sequence of SEQ ID NO: 10.

FIG. 2G shows a nucleotide sequence of SEQ ID NO: 12 that encodes the GLP-2 peptibody of SEQ ID NO: 11.

FIG. 2H shows both the nucleotide sequence of SEQ ID NO: 12 and the amino acid sequence of SEQ ID NO: 11.

FIG. 3A shows the amino acid sequence of SEQ ID NO: 13 in which there is no linker between GLP-2[A2G] and the Fc region of IgG1. The GLP-2 sequence is underlined. The GLP-2 peptibody A has the amino acid sequence set forth in SEQ ID NO: 13.

FIG. 3B shows the amino acid sequence of SEQ ID NO: 14, which has a signal peptide sequence fused to the N-terminus of the amino acid sequence of SEQ ID NO: 13.

FIG. 3C shows a nucleotide sequence of SEQ ID NO: 15 that encodes the GLP-2 peptibody of SEQ ID NO: 14.

FIG. 3D shows both the nucleotide sequence of SEQ ID NO: 15 and the amino acid sequence of SEQ ID NO: 14.

FIG. 4A shows the amino acid sequence of SEQ ID NO: 16. The GLP-2 sequence is underlined and the linker is bolded. The GLP-2 peptibody E has the amino acid sequence set forth in SEQ ID NO: 16.

FIG. 4B shows the amino acid sequence of SEQ ID NO: 17, which has a signal peptide sequence fused to the N-terminus of the amino acid sequence of SEQ ID NO: 16.

FIG. 4C shows a nucleotide sequence of SEQ ID NO: 18, that encodes the GLP-2 peptibody of SEQ ID NO: 17.

FIG. 4D shows both the nucleotide sequence of SEQ ID NO: 18 and the amino acid sequence of SEQ ID NO: 17.

FIG. 5A shows the amino acid sequence of SEQ ID NO: 19. The GLP-2 sequence is underlined and the linker is bolded. The GLP-2 peptibody J has the amino acid sequence set forth in SEQ ID NO: 19.

FIG. 5B shows the amino acid sequence of SEQ ID NO: 20, which has a signal peptide sequence fused to the N-terminus of the amino acid sequence of SEQ ID NO: 19.

FIG. 5C shows a nucleotide sequence of SEQ ID NO: 21 that encodes the GLP-2 peptibody of SEQ ID NO: 20.

FIG. 5D shows both the nucleotide sequence of SEQ ID NO: 21 and the amino acid sequence of SEQ ID NO: 20.

FIG. 6A shows the amino acid sequence of SEQ ID NO: 22. The GLP-2 sequence is underlined and the linker is bolded. The GLP-2 peptibody L has the amino acid sequence set forth in SEQ ID NO: 22.

FIG. 6B shows the amino acid sequence of SEQ ID NO: 23, which has a signal peptide sequence fused to the N-terminus of the amino acid sequence of SEQ ID NO: 22.

FIG. 6C shows a nucleotide sequence of SEQ ID NO: 24 that encodes the GLP-2 peptibody of SEQ ID NO: 23.

FIG. 6D shows both the nucleotide sequence of SEQ ID NO: 24 and the amino acid sequence of SEQ ID NO: 23.

FIG. 7A shows the amino acid sequence of SEQ ID NO: 25. The GLP-2 sequence is underlined and the linker is bolded. The GLP-2 peptibody M has the amino acid sequence set forth in SEQ ID NO: 25.

FIG. 7B shows the amino acid sequence of SEQ ID NO: 26, which has a signal peptide sequence fused to the N-terminus of the amino acid sequence of SEQ ID NO: 25.

FIG. 7C shows a nucleotide sequence of SEQ ID NO: 27 that encodes the GLP-2 peptibody of SEQ ID NO: 25.

FIG. 7D shows both the nucleotide sequence of SEQ ID NO: 27 and the amino acid sequence of SEQ ID NO: 25.

FIG. 7E shows the amino acid sequence of SEQ ID NO: 28, which is a fusion protein between GLP-2, a linker, and amino acids 25-609 of human serum albumin. The GLP-2 sequence is underlined and the linker is bolded. The GLP-2 peptibody 0 has the amino acid sequence set forth in SEQ ID NO: 28.

FIG. 7F shows the amino acid sequence of SEQ ID NO: 29, which has a signal peptide sequence fused to the N-terminus of the amino acid sequence of SEQ ID NO: 28.

FIG. 7G shows the amino acid sequence of SEQ ID NO: 30, which is a fusion protein between GLP-2, a linker that is also a GLP-2 sequence, and amino acids 25-609 of human serum albumin. The GLP-2 sequence is underlined and the linker is bolded. The GLP-2 peptibody P has the amino acid sequence set forth in SEQ ID NO: 30.

FIG. 7H shows the amino acid sequence of SEQ ID NO: 31, which has a signal peptide sequence fused to the N-terminus of the amino acid sequence of SEQ ID NO: 30.

FIGS. 8A-8D show the results of a SEC-MALS analysis (8A and 8C-8D), EM analysis (8B) of GLP-2 peptibodies B264, K and K274.

FIGS. 9A-9B show AUC analysis of GLP-2 peptibody K.

FIG. 9C shows results of a microscale thermophoresis (MST) analysis of GLP-2 peptibodies B264 and K274.

FIG. 9D shows a model of a GLP-2 peptibody and the tryptophan residues whose fluorescence is assayed under a nano differential scanning fluorimetry (NanoDSF).

FIGS. 9E and 9F show results of a nano differential scanning fluorimetry (NanoDSF) analysis of GLP-2 peptibodies B and K.

FIG. 10A shows predicted and observed results of a pharmacokinetics analysis of GLP-2 peptibody K274 in CD1 mice. FIGS. 10B and 10C show a comparison of pharmacokinetics parameters between GLP-2 peptibody K and GLP-2 peptibody K274.

FIGS. 11A-11C show the results of pharmacokinetic studies of teduglutide, GLP-2 peptibody B and GLP-2 peptibody K in cynomolgus monkeys with citrulline assayed as a biomarker.

FIGS. 12A-12C show the results of a pharmacokinetic plateau study of GLP-2 peptibody K274 with small intestine and colon weights, normalized to body weight, as endpoints.

FIGS. 13A and 13B show persistence of changed small intestine weight after dosing of GLP-2 peptibody K274 ends. FIG. 13C shows the staining of Ki67 marker of cell growth in villi and crypts of GLP-2 peptibody K274-treated intestinal cells, as compared to vehicle alone. FIG. 13D shows dose response and washout experiments measuring Ki67 marker positivity with respect to the amount of GLP-2 peptibody K274 administered. FIGS. 13E-G show results of histology studies of GLP-2 peptibody K274 effect on villi length.

FIGS. 14A-14C show the results of Ki67 marker assay of cell growth in villi and crypts of vehicle-treated and GLP-2[A2G]-treated intestinal cells. FIGS. 14D-H show results of histology studies of GLP-2[A2G] effect on villi length and crypt depth.

FIGS. 15A-15E show the effect of small intestine weight after dosing of the GLP-2 peptibody B264.

FIG. 16 shows the relative change in small intestine weight for both GLP-2 peptibody B264 and GLP-2 peptibody K274.

FIG. 17A shows the staining of Ki67 marker of cell growth in villi and crypts of GLP-2 peptibody B264-treated intestinal cells, as compared to GLP-2[A2G] treated cells. FIG. 17B shows dose response and washout experiments measuring Ki67 marker positivity with respect to the amount of GLP-2 peptibody B264 administered. FIGS. 17C-17G show results of histology studies of the effects of each of GLP-2[A2G] and GLP-2 peptibody B264 on villi length and crypt depth.

FIG. 18 shows a comparison of villi length between GLP-2 peptibody B264 and GLP-2 peptibody K274 at various doses.

FIG. 19 shows a comparison of villi length between GLP-2 peptibody B264 and GLP-2 peptibody K274 at various times during a washout period after the dosing regimen concluded. GLP-2 peptibody K274 exhibits more persistence than does GLP-2 peptibody B264.

FIG. 20A shows a comparison between the GLP-2 peptibody B264 and GLP-2 peptibody K274 concentration over a 14 day Q3D dosing regimen. FIG. 20B shows a summary of pharmacokinetics data on GLP-2 peptibody B264 and GLP-2 peptibody K274 in the mouse.

FIG. 20C shows a comparison of villus length between GLP-2 peptibody B264 and GLP-2 peptibody K274 at various doses. FIG. 20D shows a comparison of villus length between GLP-2 peptibody B264 and GLP-2 peptibody K274 at various concentrations. FIG. 20E shows a comparison between GLP-2 peptibody B264 and GLP-2 peptibody K274 effect on small intestine weight at various doses.

FIG. 21 shows the results of a triglyceride tolerance test in mice administered GLP-2 peptibody K274 and challenged with an olive oil bolus. GLP-2 peptibody K274 improved absorption of the fatty acids in olive oil, as indicated by the significantly higher postprandial triglyceride concentration in the bloodstream of the mice treated with GLP-2 peptibody K274 as compared to those not so treated.

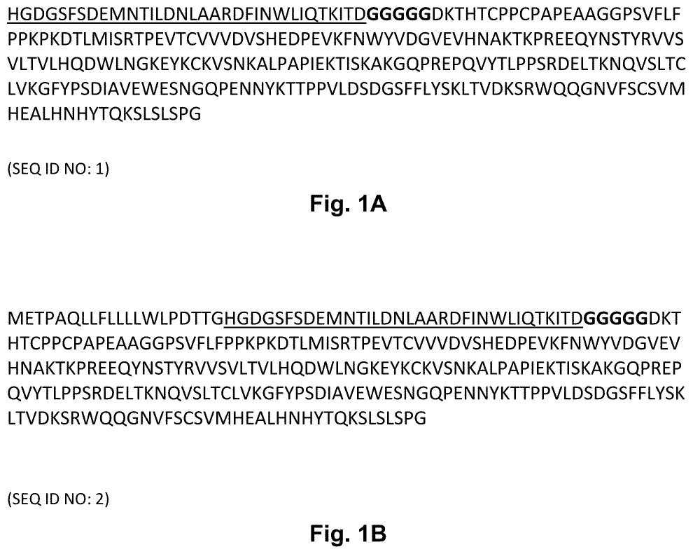

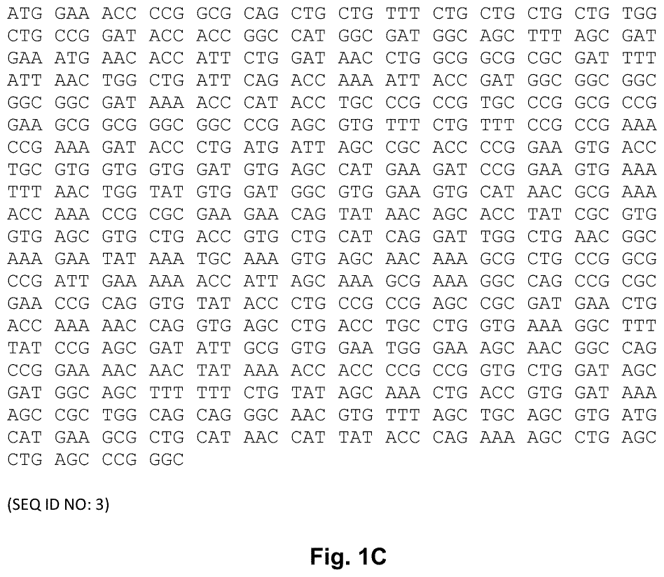

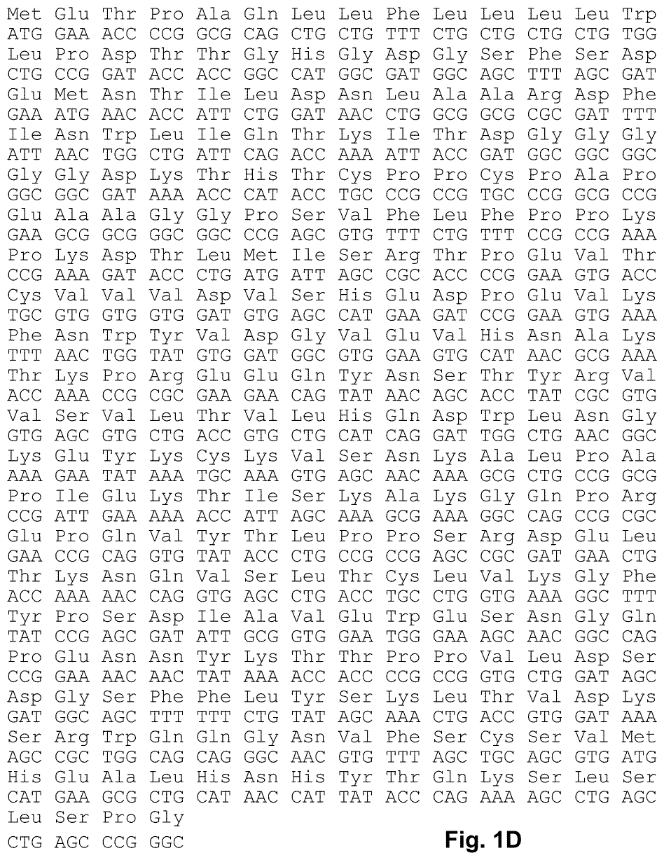

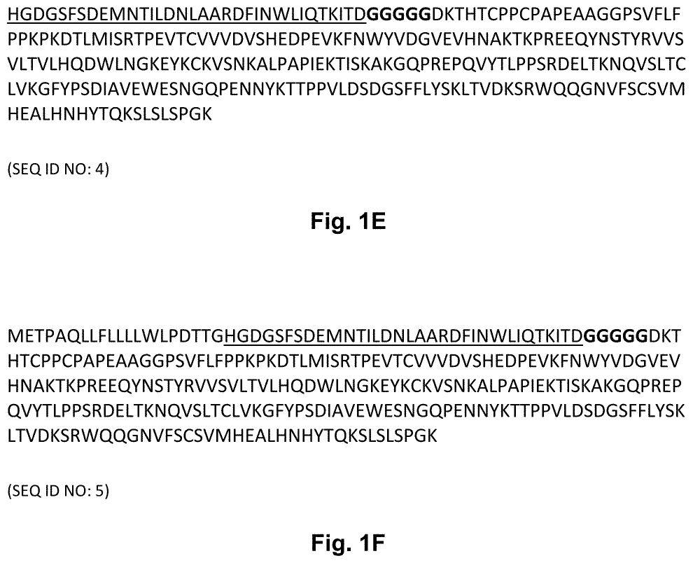

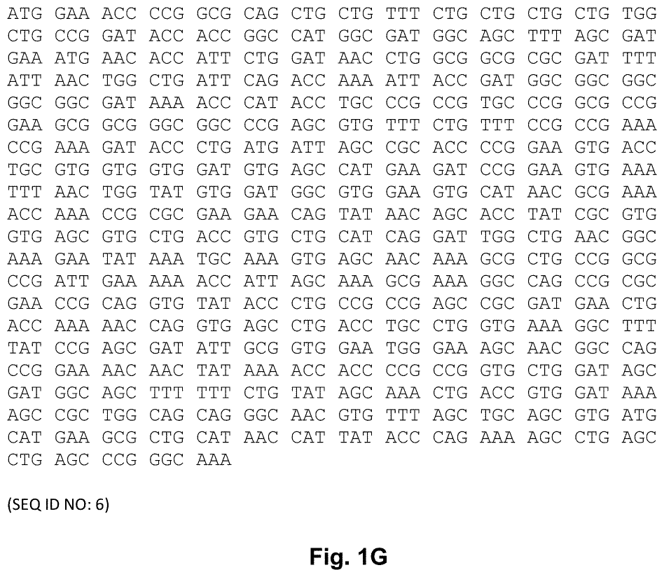

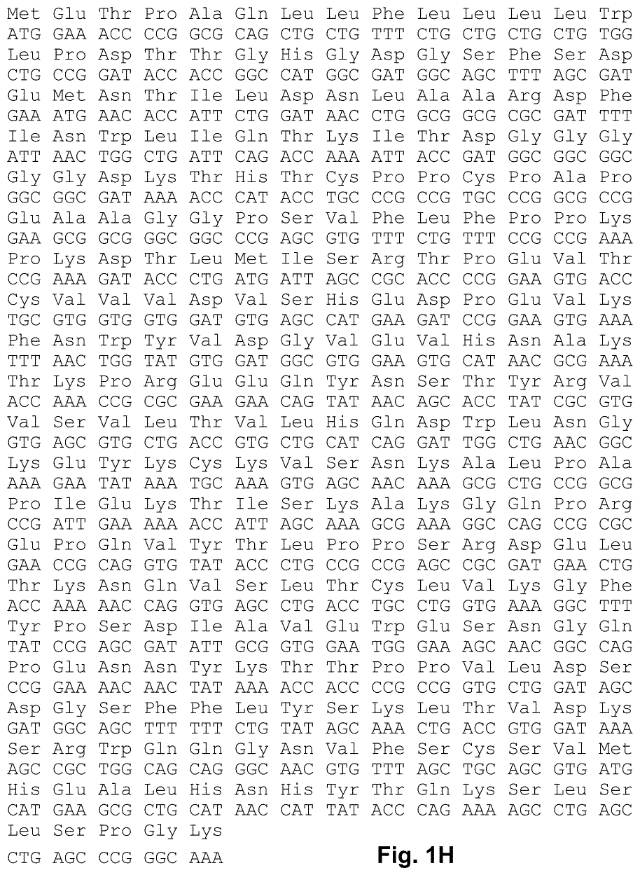

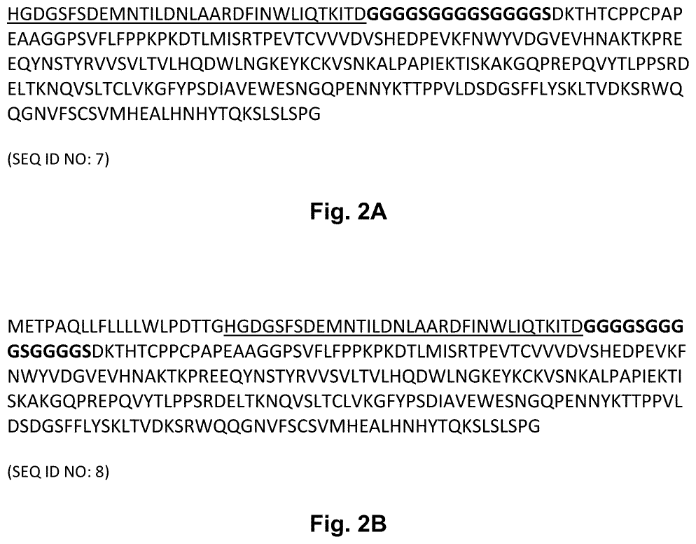

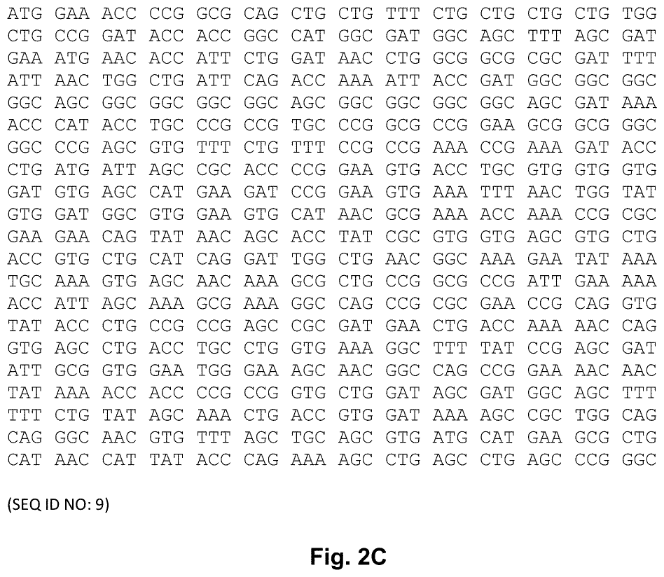

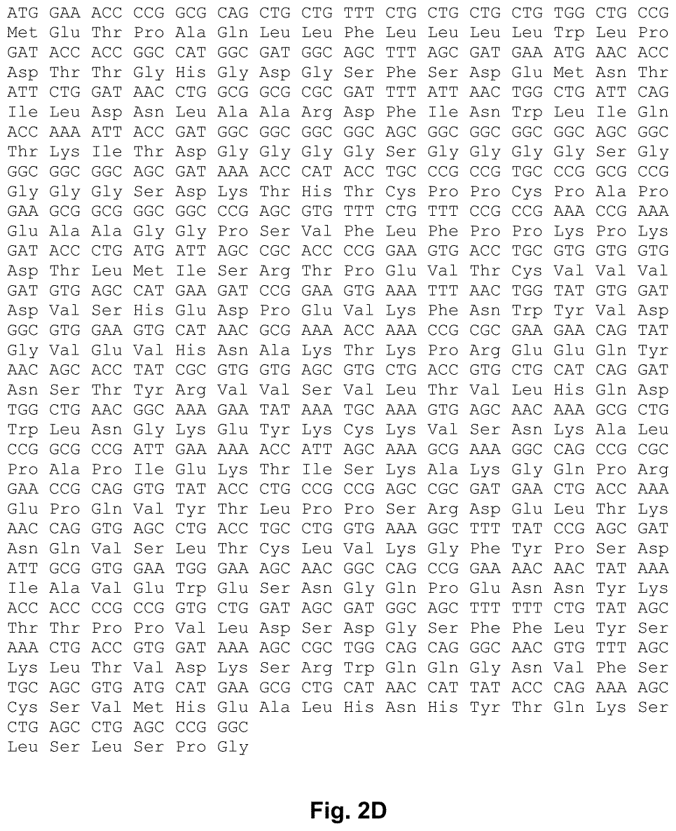

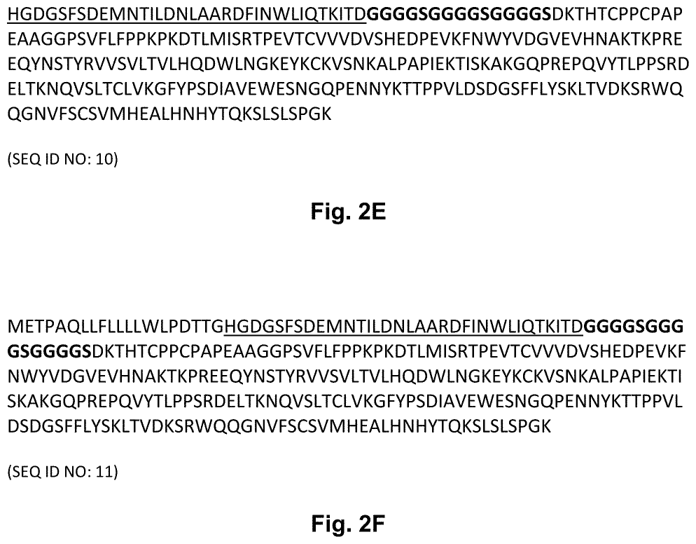

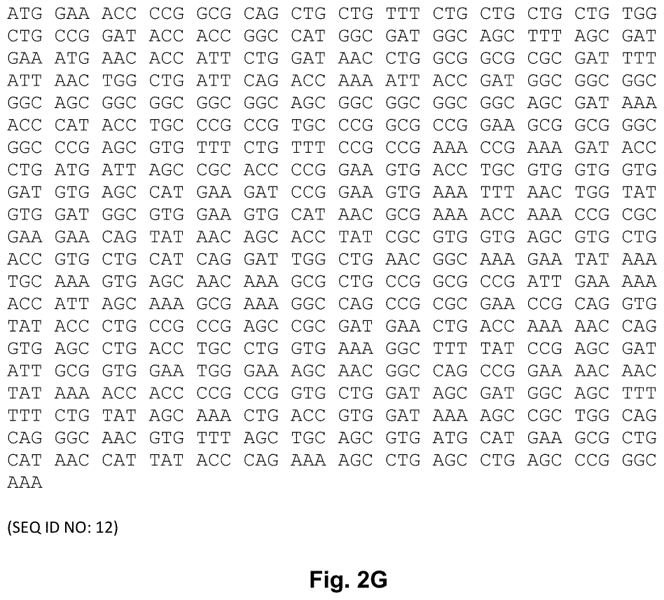

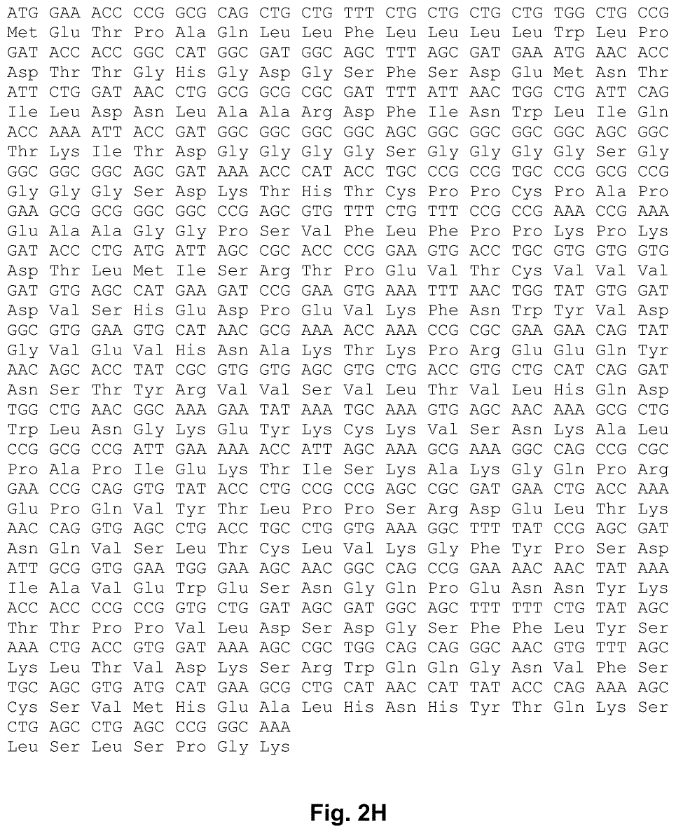

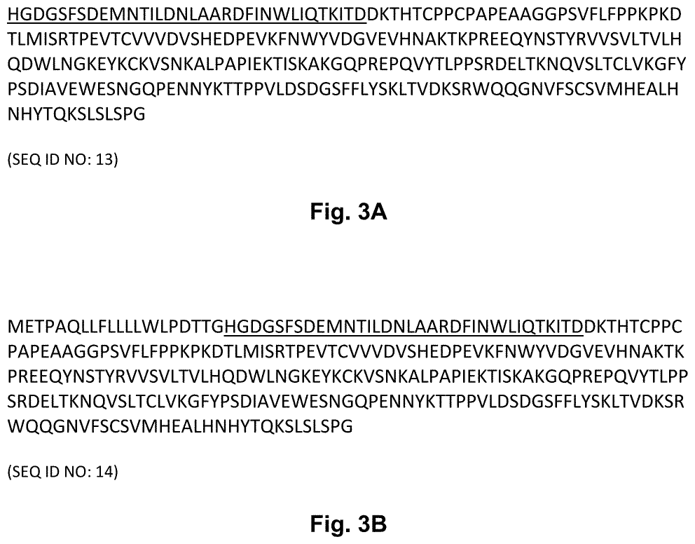

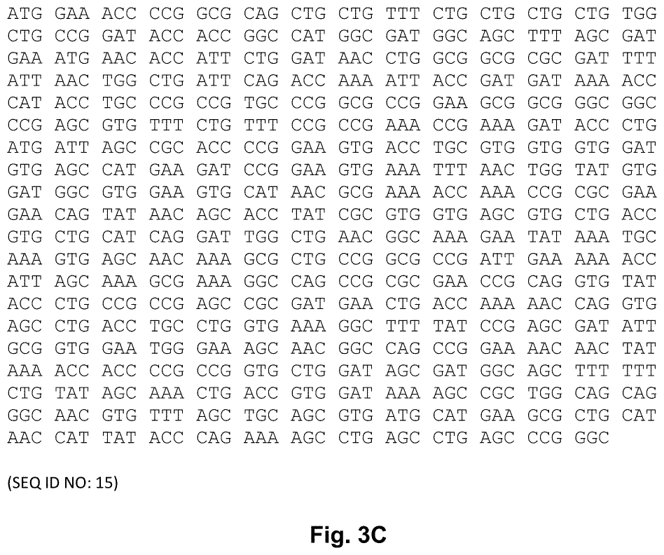

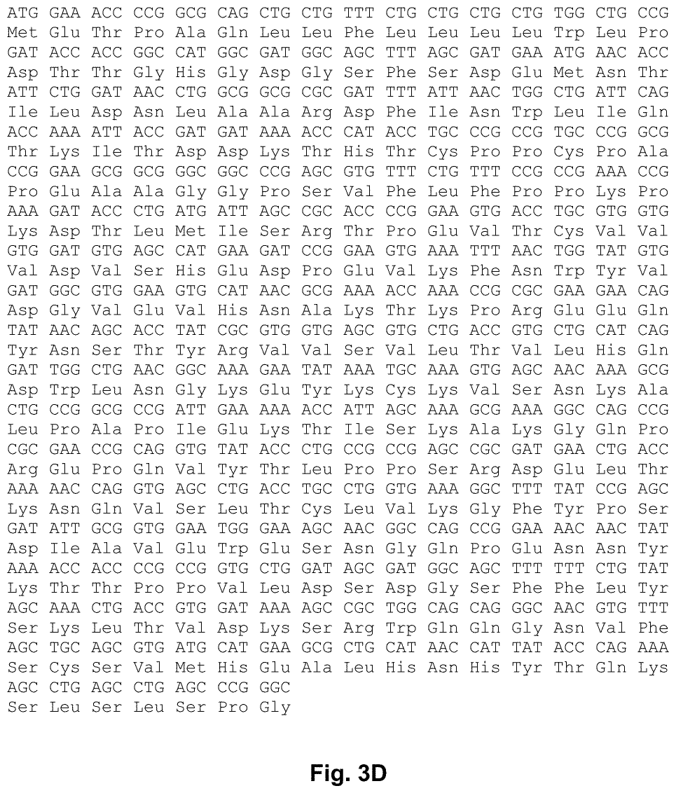

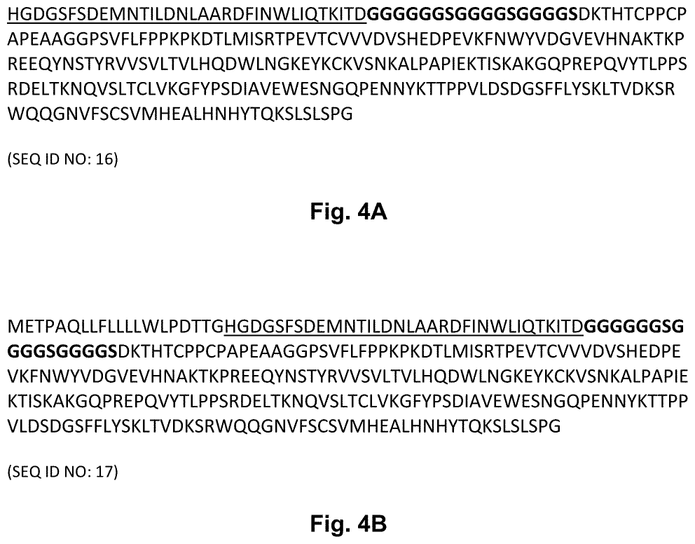

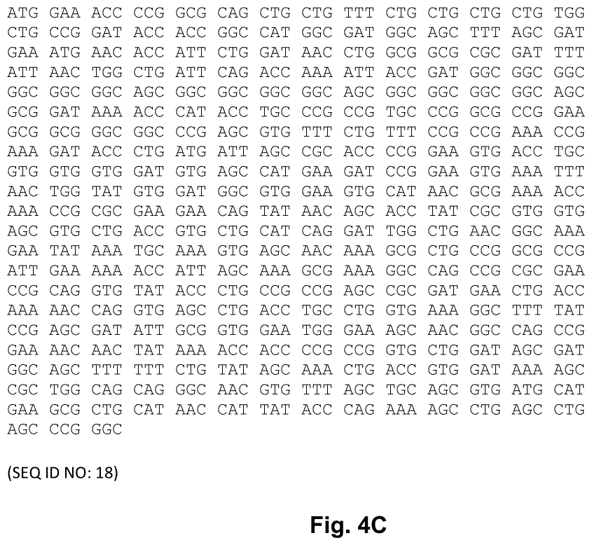

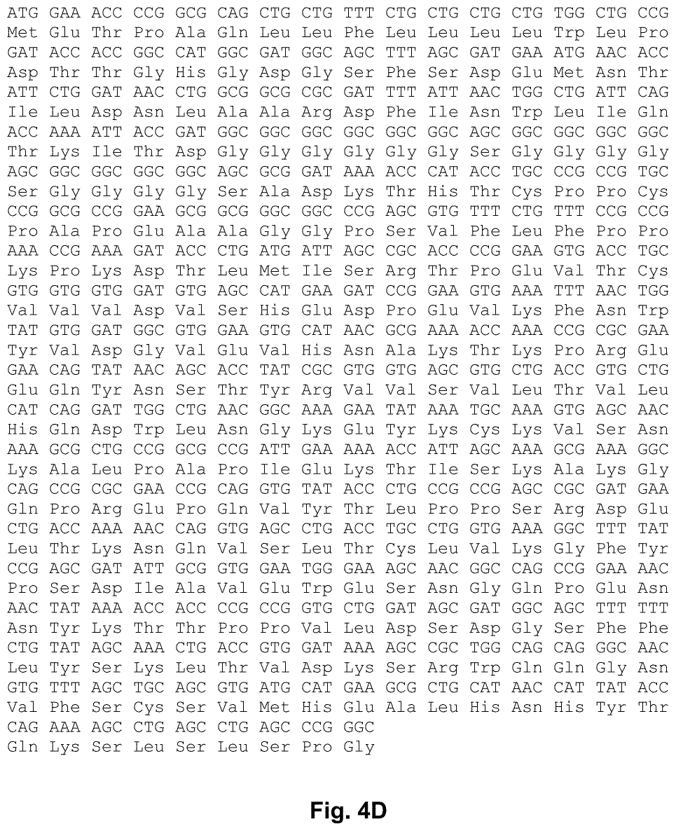

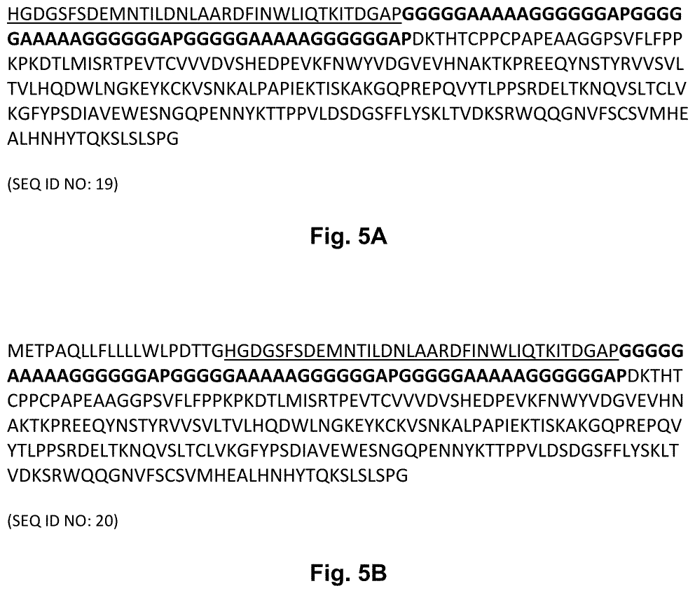

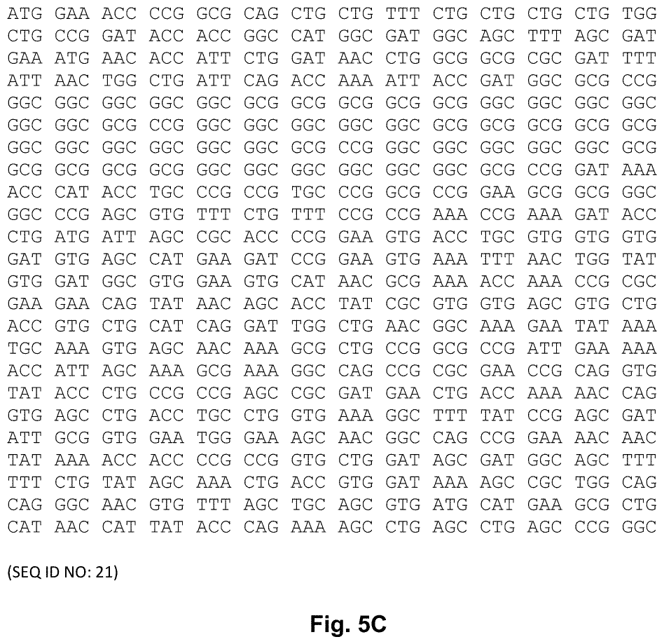

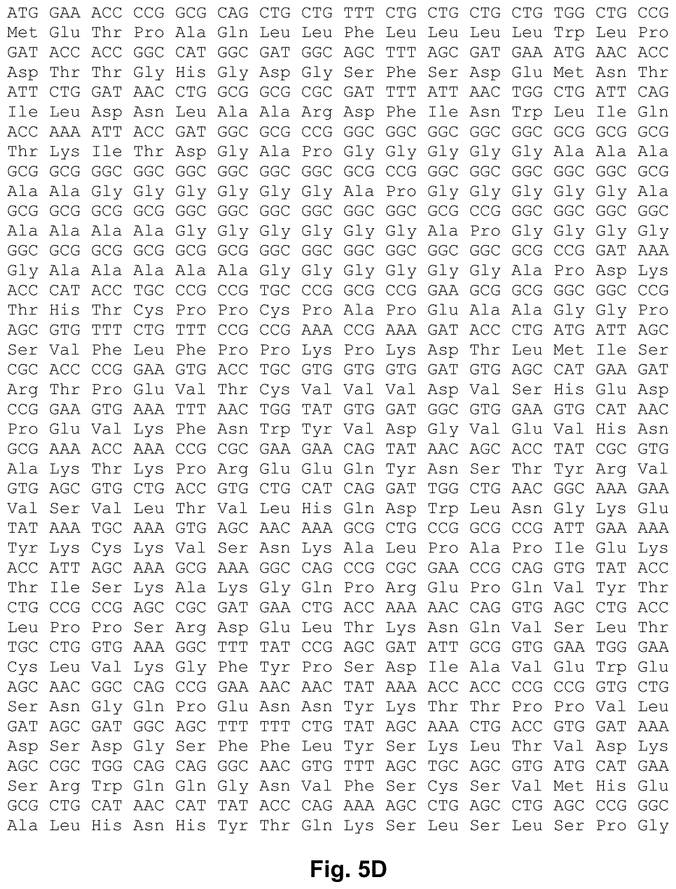

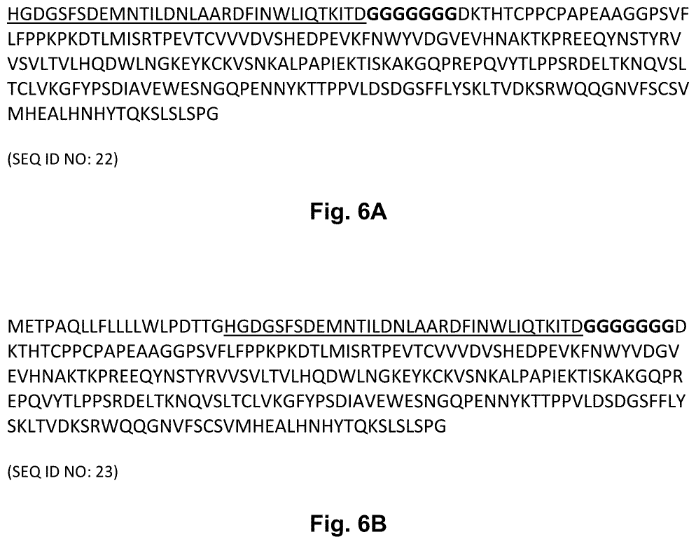

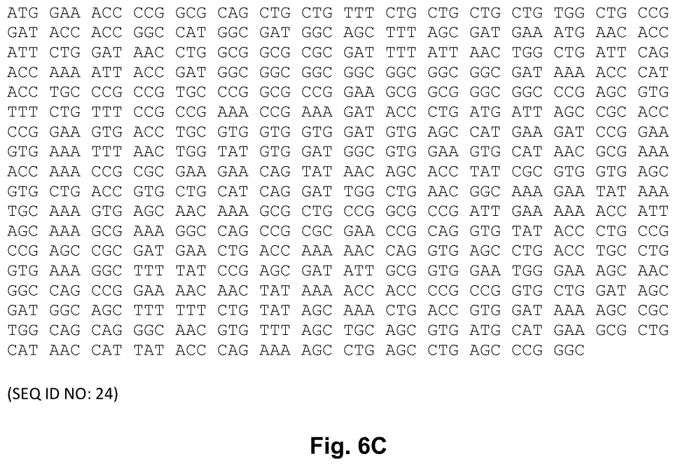

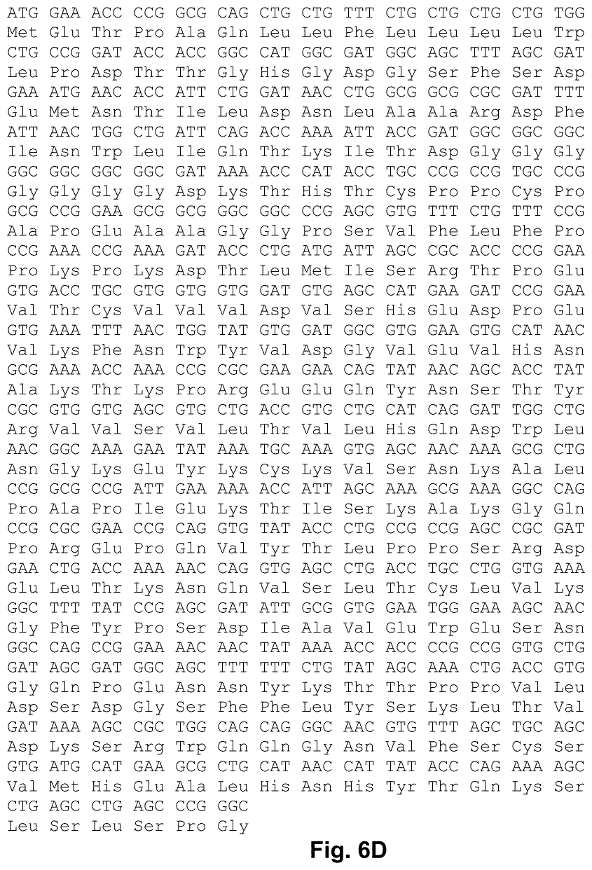

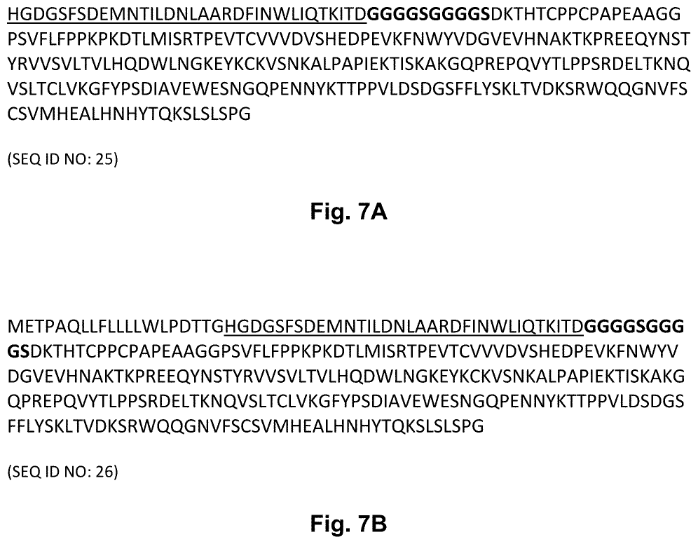

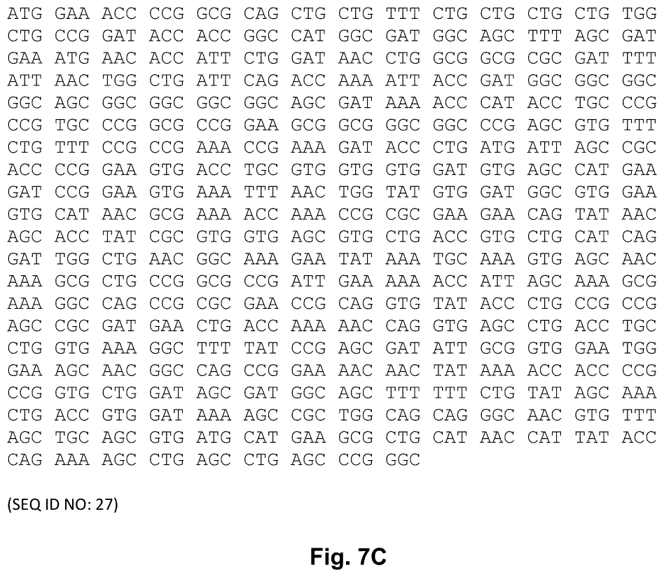

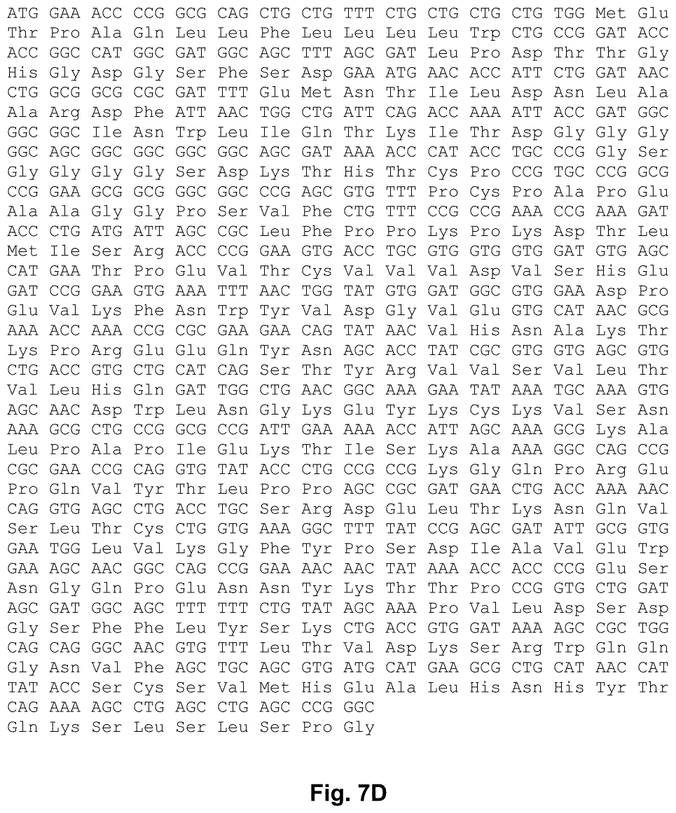

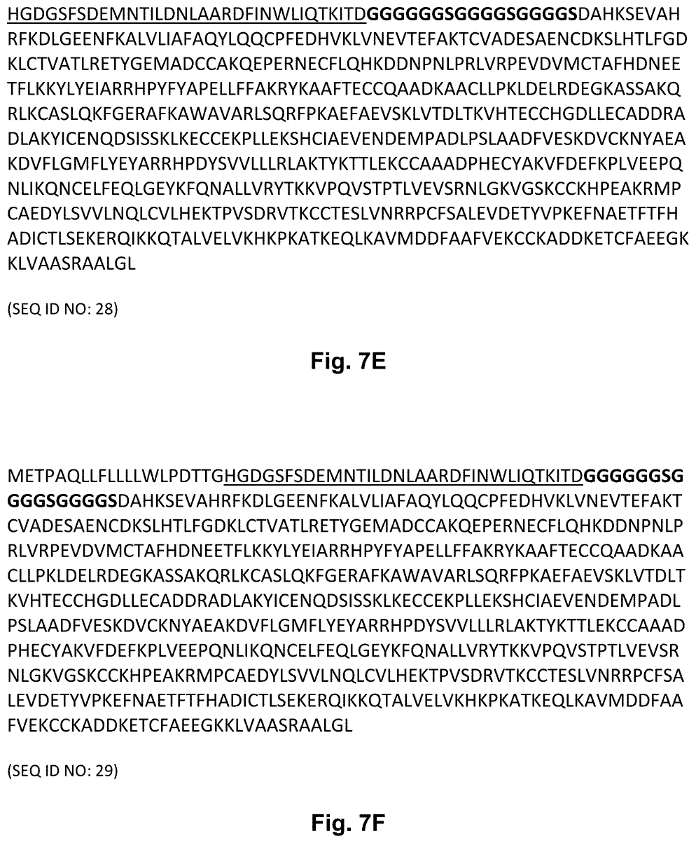

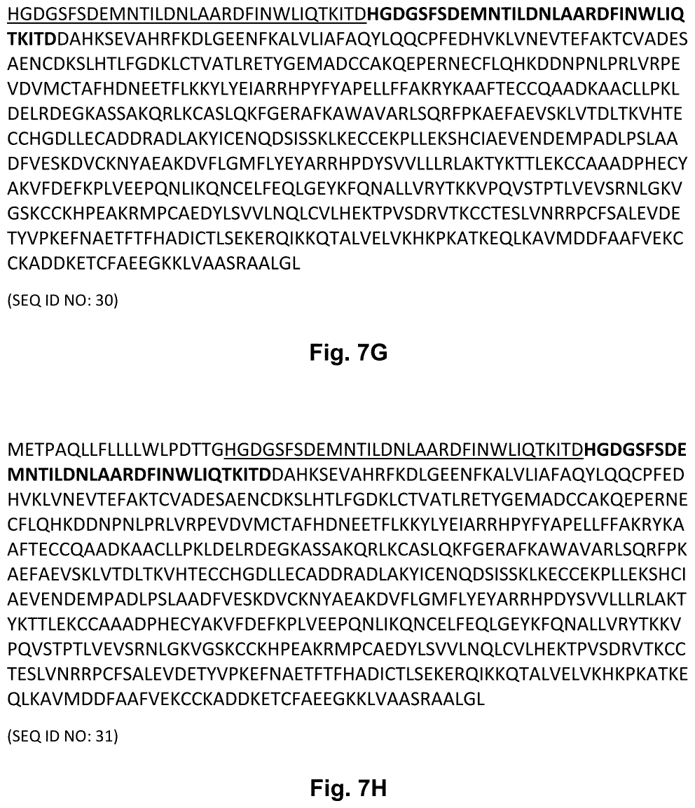

### DEFINITIONS

Unless defined otherwise, technical and scientific terms used herein have the same meaning as commonly understood by one of ordinary skill in the art to which this invention belongs. Additional definitions for the following terms and other terms are set forth throughout the specification.

The terms “a,” “an,” and “the” do not denote a limitation of quantity, but rather denote the presence of “at least one” of the referenced item.

As used in this application, the terms “about” and “approximately” are used as equivalents. Any numerals used in this application with or without about/approximately are meant to cover any normal fluctuations appreciated by one of ordinary skill in the relevant art. As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the terms “carrier” and “diluent” refers to a pharmaceutically acceptable (e.g., safe and non-toxic for administration to a human) carrier or diluting substance useful for the preparation of a pharmaceutical formulation. Exemplary diluents include sterile water, bacteriostatic water for injection (BWFI), a pH buffered solution (e.g. phosphate-buffered saline), sterile saline solution, Ringer's solution or dextrose solution.

As used herein, the term “fusion protein” or “chimeric protein” refers to a protein created through the joining of two or more originally separate proteins, or portions thereof. In some embodiments, a linker or spacer will be present between each protein.

As used herein, the term “half-life” is the time required for a quantity such as protein concentration or activity to fall to half of its value as measured at the beginning of a time period.

A “GLP-2 peptibody,” “GLP-2 peptibody portion,” or “GLP-2 peptibody fragment” and/or “GLP-2 peptibody variant” and the like can have, mimic or simulate at least one biological activity, such as but not limited to ligand binding, in vitro, in situ and/or preferably in vivo, of at least one GLP-2 peptide. For example, a suitable GU-2 peptibody, specified portion, or variant can also modulate, increase, modify, activate, at least one GLP-2 receptor signaling or other measurable or detectable activity. GLP-2 peptibodies may have suitable affinity-binding to protein ligands, for example, GLP-2 receptors, and optionally have low toxicity. The GLP-2 peptibodies can be used to treat patients for extended periods with good to excellent alleviation of symptoms and low toxicity.

As used herein, the terms “improve,” “increase” or “reduce,” or grammatical equivalents, indicate values that are relative to a baseline measurement, such as a measurement in the same individual prior to initiation of the treatment described herein, or a measurement in a control subject (or multiple control subject) in the absence of the treatment described herein. A “control subject” is a subject afflicted with the same form of disease as the subject being treated, who is about the same age as the subject being treated.

As used herein, the term “in vitro” refers to events that occur in an artificial environment, e.g., in a test tube or reaction vessel, in cell culture, etc., rather than within a multi-cellular organism.

As used herein, the term “in vivo” refers to events that occur within a multi-cellular organism, such as a human and a non-human animal. In the context of cell-based systems, the term may be used to refer to events that occur within a living cell (as opposed to, for example, in vitro systems).

As used herein, the term “linker” refers to, in a fusion protein, an amino acid sequence other than that appearing at a particular position in the natural protein and is generally designed to be flexible or to interpose a structure, such as an α-helix, between two protein moieties. A linker is also referred to as a spacer. A linker or a spacer typically does not have biological function on its own.

As used herein, the phrase “pharmaceutically acceptable” refers to molecular entities and compositions that are generally regarded as physiologically tolerable.

The term “polypeptide” as used herein refers to a sequential chain of amino acids linked together via peptide bonds. The term is used to refer to an amino acid chain of any length, but one of ordinary skill in the art will understand that the term is not limited to lengthy chains and can refer to a minimal chain comprising two amino acids linked together via a peptide bond. As is known to those skilled in the art, polypeptides may be processed and/or modified. As used herein, the terms “polypeptide” and “peptide” are used inter-changeably. The term “polypeptide” can also refer to proteins.

As used herein, the term “prevent” or “prevention”, when used in connection with the occurrence of a disease, disorder, and/or condition, refers to reducing the risk of developing the disease, disorder and/or condition. See the definition of “risk.”

As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### DETAILED DESCRIPTION OF THE INVENTION

Various aspects of the invention are described in detail in the following sections. The use of sections is not meant to limit the invention. Each section can apply to any aspect of the invention.

Various GLP-2 peptibodies described herein comprise a linker between the GLP-2 sequence and the Fc, or Fc variant, sequence. Alternatively, an albumin sequence may be used instead of an Fc or Fc variant sequence. A linker provides structural flexibility by allowing the peptibody to have alternative orientations and binding properties. The linker is preferably made up of amino acids linked together by peptide bonds. Some of these amino acids may be glycosylated, as is well understood by those in the art. The amino acids may be selected from glycine, alanine, serine, proline, asparagine, glutamine, and lysine. Even more preferably, a linker is made up of a majority of amino acids that are sterically unhindered, such as glycine, serine and alanine.

The GLP-2 sequence may be directly or indirectly linked to an Fc domain, or an albumin domain. In one embodiment, the linker has the sequence GGGGG (e.g., in a GLP-2 peptibody comprising sequence of SEQ ID NO: 1).

In another embodiment, the linker has the sequence GGGGSGGGGSGGGGS (e.g., in GLP-2 peptibody comprising sequence of SEQ ID NO: 7).

In another embodiment, the linker has the sequence GGGGGGSGGGGSGGGGSA (e.g., in GLP-2 peptibody comprising sequence of SEQ ID NO: 16).

In another embodiment, the linker has the sequence GAPGGGGGAAAAAGGGGGGAPGGGGGAAAAAGGGGGGAPGGGGGAAAAAGGGGG GAP (e.g., in GLP-2 peptibody comprising sequence of SEQ ID NO: 19).

In another embodiment, the linker has the sequence GGGGGGG (e.g., in GLP-2 peptibody comprising sequence of SEQ ID NO: 22).

In another embodiment, the linker has the sequence GGGGSGGGGS (e.g., in GLP-2 peptibody comprising sequence of SEQ ID NO: 25).

Suitable linkers or spacers also include those having an amino acid sequence at least 50%, 55%, 60%, 65%, 70%, 75%, 80%, 85%, 90%, 91%, 92%, 93%, 94%, 95%, 96%, 97%, 98%, 99% or more homologous or identical to the above exemplary linkers. Additional linkers suitable for use with some embodiments may be found in US2012/0232021, filed on Mar. 2, 2012, the disclosure of which is hereby incorporated by reference in its entirety.

In various embodiments, the GLP-2[A2G] sequence is used for GLP-2. In the GLP-2[A2G] sequence, there is a glycine at position 2 instead of an alanine.

In one embodiment, the GLP-2 peptibody has the following formula:

GLP-2[A2G]-linker-albumin(25-609)

The linker has the sequence GGGGGGSGGGGSGGGGSA (e.g., in GLP-2 peptibody comprising sequence of SEQ ID NO: 28).

In another embodiment, the GLP-2 peptibody has the following formula:

(GLP-2[A2G])₂-albumin(25-609)

In one aspect is provided a glucagon-like peptide (GLP-2) peptibody selected from:
a) a GLP-2 peptibody comprising the amino acid sequence of

(SEQ ID NO: 1)
HGDGSFSDEMNTILDNLAARDFINWLIQTKITDGGGGGDKTHTCPPCPA
PEAAGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSV
MHEALHNHYTQKSLSLSPG,

b) a GLP-2 peptibody comprising the amino acid sequence of

(SEQ ID NO: 7)
HGDGSFSDEMNTILDNLAARDFINWLIQTKITDGGGGSGGGGSGGGGSD
KTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRW
QQGNVFSCSVMHEALHNHYTQKSLSLSPG,

c) a GLP-2 peptibody comprising the amino acid sequence of

(SEQ ID NO: 13)
HGDGSFSDEMNTILDNLAARDFINWLIQTKITDDKTHTCPPCPAPEAAG
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEAL
HNHYTQKSLSLSPG,

d) a GLP-2 peptibody comprising the amino acid sequence of

(SEQ ID NO: 16)
HGDGSFSDEMNTILDNLAARDFINWLIQTKITDGGGGGGSGGGGSGGGG
SDKTHTCPPCPAPEAAGGPSVFLFPPKPKDTLMISRTPEVTCVVVDVSH
EDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKS
RWQQGNVFSCSVMHEALHNHYTQKSLSLSPG,

e) a GLP-2 peptibody comprising the amino acid sequence of

(SEQ ID NO: 19)
HGDGSFSDEMNTILDNLAARDFINWLIQTKITDGAPGGGGGAAAAAGGG
GGGAPGGGGGAAAAAGGGGGGAPGGGGGAAAAAGGGGGGAPDKTHTCPP
CPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFS
CSVMHEALHNHYTQKSLSLSPG,

f) a GLP-2 peptibody comprising the amino acid sequence of

(SEQ ID NO: 22)
HGDGSFSDEMNTILDNLAARDFINWLIQTKITDGGGGGGGDKTHTCPPC
PAPEAAGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSC
SVMHEALHNHYTQKSLSLSPG,

g) a GLP-2 peptibody comprising the amino acid sequence of

(SEQ ID NO: 25)
HGDGSFSDEMNTILDNLAARDFINWLIQTKITDGGGGSGGGGSDKTHTC
PPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNV
FSCSVMHEALHNHYTQKSLSLSPG,

h) a GLP-2 peptibody comprising the amino acid sequence of

(SEQ ID NO: 28)
HGDGSFSDEMNTILDNLAARDFINWLIQTKITDGGGGGGSGGGGSGGGG
SDAHKSEVAHRFKDLGEENFKALVLIAFAQYLQQCPFEDHVKLVNEVTE
FAKTCVADESAENCDKSLHTLFGDKLCTVATLRETYGEMADCCAKQEPE
RNECFLQHKDDNPNLPRLVRPEVDVMCTAFHDNEETFLKKYLYEIARRH
PYFYAPELLFFAKRYKAAFTECCQAADKAACLLPKLDELRDEGKASSAK
QRLKCASLQKFGERAFKAWAVARLSQRFPKAEFAEVSKLVTDLTKVHTE
CCHGDLLECADDRADLAKYICENQDSISSKLKECCEKPLLEKSHCIAEV
ENDEMPADLPSLAADFVESKDVCKNYAEAKDVFLGMFLYEYARRHPDYS
VVLLLRLAKTYKTTLEKCCAAADPHECYAKVFDEFKPLVEEPQNLIKQN
CELFEQLGEYKFQNALLVRYTKKVPQVSTPTLVEVSRNLGKVGSKCCKH
PEAKRMPCAEDYLSVVLNQLCVLHEKTPVSDRVTKCCTESLVNRRPCFS
ALEVDETYVPKEFNAETFTFHADICTLSEKERQIKKQTALVELVKHKPK
ATKEQLKAVMDDFAAFVEKCCKADDKETCFAEEGKKLVAASRAALGL,

and
k) a GLP-2 peptibody comprising the amino acid sequence of

(SEQ ID NO: 30)
HGDGSFSDEMNTILDNLAARDFINWLIQTKITDHGDGSFSDEMNTILDN
LAARDFINWLIQTKITDDAHKSEVAHRFKDLGEENFKALVLIAFAQYLQ
QCPFEDHVKLVNEVTEFAKTCVADESAENCDKSLHTLFGDKLCTVATLR
ETYGEMADCCAKQEPERNECFLQHKDDNPNLPRLVRPEVDVMCTAFHDN
EETFLKKYLYEIARRHPYFYAPELLFFAKRYKAAFTECCQAADKAACLL
PKLDELRDEGKASSAKQRLKCASLQKFGERAFKAWAVARLSQRFPKAEF
AEVSKLVTDLTKVHTECCHGDLLECADDRADLAKYICENQDSISSKLKE
CCEKPLLEKSHCIAEVENDEMPADLPSLAADFVESKDVCKNYAEAKDVF
LGMFLYEYARRHPDYSVVLLLRLAKTYKTTLEKCCAAADPHECYAKVFD
EFKPLVEEPQNLIKQNCELFEQLGEYKFQNALLVRYTKKVPQVSTPTLV
EVSRNLGKVGSKCCKHPEAKRMPCAEDYLSVVLNQLCVLHEKTPVSDRV
TKCCTESLVNRRPCFSALEVDETYVPKEFNAETFTFHADICTLSEKERQ
IKKQTALVELVKHKPKATKEQLKAVMDDFAAFVEKCCKADDKETCFAEE
GKKLVAASRAALGL;

In some embodiments, the GLP-2 peptibody comprises the amino acid sequence of

(SEQ ID NO: 1)
HGDGSFSDEMNTILDNLAARDFINWLIQTKITDGGGGGDKTHTCPPCPA
PEAAGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSV
MHEALHNHYTQKSLSLSPG,

or a pharmaceutically acceptable salt thereof.

In some embodiments, the GLP-2 peptibody comprises the amino acid sequence of

(SEQ ID NO: 7)
HGDGSFSDEMNTILDNLAARDFINWLIQTKITDGGGGSGGGGSGGGGSD
KTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRW
QQGNVFSCSVMHEALHNHYTQKSLSLSPG,

or a pharmaceutically acceptable salt thereof.

It is contemplated that improved binding between Fc domain and the FcRn receptor results in prolonged serum half-life. Thus, in some embodiments, a suitable Fc domain comprises one or more amino acid mutations that lead to improved binding to FcRn. Various mutations within the Fc domain that effect improved binding to FcRn are known in the art and can be adapted to practice the present invention. In some embodiments, a suitable Fc domain comprises one or more mutations at one or more positions corresponding to Thr 250, Met 252, Ser 254, Thr 256, Thr 307, Glu 380, Met 428, His 433, and/or Asn 434 of human IgG1.

GLP-2 peptibodies of the present invention can provide at least one suitable property as compared to known proteins, such as, but not limited to, at least one of increased half-life, increased activity, more specific activity, increased avidity, increased or decreased off rate, a selected or more suitable subset of activities, less immunogenicity, increased quality or duration of at least one desired therapeutic effect, less side effects, and the like.

Typically, a suitable GLP-2 peptibody, e.g., a GLP-2 peptibody comprising the amino acid sequence of SEQ ID NO: 1 or SEQ ID NO: 7, has an in vivo half-life of or greater than about 2 hours, 3 hours, 4 hours, 6 hours, 8 hours, 10 hours, 12 hours, 14 hours, 16 hours, 18 hours, 20 hours, 22 hours, 24 hours, 26 hours, 28 hours, 30 hours, 32 hours, 34 hours, 36 hours, 38 hours, 40 hours, 42 hours, 44 hours, 46 hours, or 48 hours. In some embodiments, a recombinant GLP-2 peptibody has an in vivo half-life of between 2 and 48 hours, between 2 and 44 hours, between 2 and 40 hours, between 3 and 36 hours, between 3 and 32 hours, between 3 and 28 hours, between 4 and 24 hours, between 4 and 20 hours, between 6 and 18 hours, between 6 and 15 hours, and between 6 and 12 hours.

The GLP-2 peptibodies or specified portion or variants thereof may be produced by at least one cell line, mixed cell line, immortalized cell or clonal population of immortalized and/or cultured cells. Immortalized protein producing cells can be produced using suitable methods. Preferably, the at least one GLP-2 peptibody or specified portion or variant is generated by providing nucleic acid or vectors comprising DNA derived or having a substantially similar sequence to, at least one human immunoglobulin locus that is functionally rearranged, or which can undergo functional rearrangement, and which further comprises a peptibody structure as described herein.

The GLP-2 peptibodies can bind human protein ligands with a wide range of affinities (K_{D}). In a preferred embodiment, at least one human GLP-2 peptibody of the present invention can optionally bind at least one protein ligand with high affinity. For example, at least one GLP-2 peptibody of the present invention can bind at least one protein ligand with a K_{D} equal to or less than about 10⁻⁷ M or, more preferably, with a K_{D} equal to or less than about 0.1-9.9 (or any range or value therein)×10⁻⁷, 10⁻⁸, 10⁻⁹, 10⁻¹⁰, 10⁻¹¹, 10⁻¹², or 10⁻¹³ M, or any range or value therein.

The affinity or avidity of a GLP-2 peptibody for at least one protein ligand can be determined experimentally using any suitable method, e.g., as used for determining antibody-antigen binding affinity or avidity. (See, for example, Kuby, Janis Immunology, W. H. Freeman and Company: New York, N.Y. (1992)). The measured affinity of a particular GLP-2 peptibody-ligand interaction can vary if measured under different conditions, e.g., salt concentration and pH. Thus, measurements of affinity and other ligand-binding parameters (e.g., K_{D}, Kₐ, K_{d}) are preferably made with standardized solutions of GLP-2 peptibody and ligand, and a standardized buffer, such as the buffer described herein or known in the art.

There may or may not be a lysine (K) at the C-terminus. The GLP-2 peptibodies comprising polypeptide sequence of SEQ ID NOS: 1, 7, 13, 16, 19, 22 and 25 lack the C-terminal lysine. In particular, the amino acid sequences of SEQ ID NO: 1 and SEQ ID NO: 7 lack the C-terminal lysine. At the same time, in any of the embodiments or aspects described herein, lysine can be added to C-terminus. For instance, the amino acid sequences of SEQ ID NO: 4 and SEQ ID NO: 10 have lysine at the C-terminus.

In any embodiment or aspect described herein, the GLP-2 peptibody is processed from a GLP-2 precursor polypeptide that comprises a signal peptide directly linked with GLP-2, with a linker between GLP-2 and an Fc region of any of IgG1, IgG2, IgG3 and IgG4. The Fc region may be IgG1 with the LALA mutation. The GLP-2 precursor polypeptide may have the following formula:

Signal peptide-GLP-2[A2G]-linker-IgG1(LALA)

LALA refers to the L234A and L235A (EU numbering) mutations in an antibody. The LALA mutations are present in the following polypeptide sequences disclosed herein, e.g. SEQ ID NOS: 1, 4, 7, 10, 13, 16, 19, 22 and 25. The LALA mutations can greatly reduce binding to Fc gamma-Rs and in turn prevent the GLP-2 peptibodies from causing unwanted antibody effector functions. See Leabman, M. K. et al., “Effects of altered Fc gammaR binding on antibody pharmacokinetics in cynomolgus monkeys” mAbs 5(6):2013.

A GLP-2 peptibody, or specified portion or variant thereof, that partially or preferably substantially provides at least one GLP-2 biological activity, can bind the GLP-2 ligand and thereby provide at least one activity that is otherwise mediated through the binding of GLP-2 to at least one ligand, such as a GLP-2 receptor, or through other protein-dependent or mediated mechanisms. As used herein, the term “GLP-2 peptibody activity” refers to a GLP-2 peptibody that can modulate or cause at least one GLP-2 dependent activity by about 20-10,000% as compared to wildtype GLP-2 peptide or a GLP-2[A2G] peptide, preferably by at least about 60, 70, 80, 90, 91, 92, 93, 94, 95, 96, 97, 98, 99, 100, 110, 120, 130, 140, 150, 160, 170, 180, 190, 200, 250, 300, 350, 400, 450, 500, 550, 600, 700, 800, 900, 1000, 2000, 3000, 4000, 5000, 6000, 7000, 8000, 9000% or more as compared to a wildtype GLP-2 peptide or a GLP-2[A2G] peptide, depending on the assay.

The capacity of a GLP-2 peptibody or specified portion or variant to provide at least one protein-dependent activity is preferably assessed by at least one suitable protein biological assay, as described herein and/or as known in the art. A human GLP-2 peptibody or specified portion or variant of the invention can be similar to any class (IgG, IgA, IgM, etc.) or isotype and can comprise at least a portion of a kappa or lambda light chain. In one embodiment, the human GLP-2 peptibody or specified portion or variant comprises IgG heavy chain CH2 and CH3 of, at least one of subclass, e.g., IgG1, IgG2, IgG3 or IgG4.

At least one GLP-2 peptibody or specified portion or variant of the invention binds at least one ligand, subunit, fragment, portion or any combination thereof. The at least one GLP-2 peptide, variant or derivative of at least one GLP-2 peptibody, specified portion or variant of the present invention can optionally bind at least one specified epitope of the ligand. The binding epitope can comprise any combination of at least one amino acid sequence of at least 1-3 amino acids to the entire specified portion of contiguous amino acids of the sequences of a protein ligand, such as a GLP-2 receptor or portion thereof.

The invention also relates to peptibodies, ligand-binding fragments and immunoglobulin chains comprising amino acids in a sequence that is substantially the same as an amino acid sequence described herein. Preferably, such peptibodies or ligand-binding fragments thereof can bind human GLP-2 ligands, such as receptors, with high affinity (e.g., K_{D} less than or equal to about 10⁻⁷ M). Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T.

As those of skill will appreciate, the present invention includes at least one biologically active GLP-2 peptibody or specified portion or variant of the present invention. In some embodiments, biologically active GLP-2 peptibodies or specified portions or variants have a specific activity at least 2%, 3%, 4%, 5%, 6%, 7%, 8%, 9%, 10%, 12%, or 15%, of that of the native (non-synthetic), endogenous or related and known inserted or fused protein or specified portion or variant.

### Nucleic Acids

In another aspect is provided a polynucleotide comprising a sequence encoding the GLP-2 peptibodies described herein. The sequence may have 70%, 75%, 80%, 85%, 90%, 91%, 92%, 93%, 94%, 95%, 96%, 97%, 98%, 99% or 100% identity to any of SEQ ID NOS: 3, 9, 15, 18, 21, 24 or 27. In some embodiments, the polynucleotide may comprise further noncoding sequence. The polynucleotides may further comprise specified fragments, variants or consensus sequences thereof, or a deposited vector comprising at least one of these sequences. The nucleic acid molecules can be in the formed of RNA, such as mRNA, hnRNA, tRNA or any other form, or in the form of DNA, including, but not limited to, cDNA and genomic DNA obtained by cloning or produced synthetically, or any combination thereof. The DNA can be triple-stranded, double-stranded or single-stranded, or any combination thereof. Any portion of at least one strand of the DNA or RNA can be the coding strand, also known as the sense strand, or it can be the noncoding strand, also referred to as the antisense strand.

In some embodiments, the nucleic acid encoding a transgene may be modified to provide increased expression of the encoded GLP-2 peptibody, which is also referred to as codon optimization. For example, the nucleic acid encoding a transgene can be modified by altering the open reading frame for the coding sequence. As used herein, the term “open reading frame” is synonymous with “ORF” and means any nucleotide sequence that is potentially able to encode a protein, or a portion of a protein. An open reading frame usually begins with a start codon (represented as, e.g. AUG for an RNA molecule and ATG in a DNA molecule in the standard code) and is read in codon-triplets until the frame ends with a STOP codon (represented as, e.g. UAA, UGA or UAG for an RNA molecule and TAA, TGA or TAG in a DNA molecule in the standard code). As used herein, the term “codon” means a sequence of three nucleotides in a nucleic acid molecule that specifies a particular amino acid during protein synthesis; also called a triplet or codon-triplet. For example, of the 64 possible codons in the standard genetic code, two codons, GAA and GAG encode the amino acid glutamine whereas the codons AAA and AAG specify the amino acid lysine. In the standard genetic code three codons are stop codons, which do not specify an amino acid. As used herein, the term “synonymous codon” means any and all of the codons that code for a single amino acid. Except for methionine and tryptophan, amino acids are coded by two to six synonymous codons. For example, in the standard genetic code the four synonymous codons that code for the amino acid alanine are GCA, GCC, GCG and GCU, the two synonymous codons that specify glutamine are GAA and GAG and the two synonymous codons that encode lysine are AAA and AAG.

A nucleic acid encoding the open reading frame of a GLP-2 peptibody may be modified using standard codon optimization methods. Various commercial algorithms for codon optimization are available and can be used to practice the present invention. Typically, codon optimization does not alter the encoded amino acid sequences.

A nucleotide change may alter a synonymous codon within the open reading frame in order to agree with the endogenous codon usage found in a particular heterologous cell selected to express a GLP-2 peptibody. Alternatively or additionally, a nucleotide change may alter the G+C content within the open reading frame to better match the average G+C content of open reading frames found in endogenous nucleic acid sequence present in the heterologous host cell. A nucleotide change may also alter a polymononucleotide region or an internal regulatory or structural site found within a GLP-2 peptibody sequence. Thus, a variety of modified or optimized nucleotide sequences are envisioned including, without limitation, nucleic acid sequences providing increased expression of GLP-2 peptibodies in a prokaryotic cell, yeast cell, insect cell, and in a mammalian cell.

As indicated herein, polynucleotides may further include additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example—ribosome binding and stability of mRNA); an additional coding sequence that codes for additional amino acids, such as those that provide additional functionalities. Thus, the sequence encoding a GLP-2 peptibody or specified portion or variant can be fused to a marker sequence, such as a sequence encoding a peptide that facilitates purification of the fused GLP-2 peptibody or specified portion or variant comprising a GLP-2 peptibody fragment or portion.

The nucleic acids may further comprise sequences in addition to a polynucleotide of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the present invention. For example, a hexa-histidine marker sequence provides a convenient means to purify the proteins of the present invention. The nucleic acid of the present invention—excluding the coding sequence—is optionally a vector, adapter, or linker for cloning and/or expression of a polynucleotide of the present invention.

The coding region of a transgene may include one or more silent mutations to optimize codon usage for a particular cell type. For example, the codons of a GLP-2 peptibody may be optimized for expression in a vertebrate cell. In some embodiments, the codons of a GLP-2 peptibody may be optimized for expression in a mammalian cell. In some embodiments, the codons of a GLP-2 peptibody may be optimized for expression in a human cell. In some embodiments, the codons of a GLP-2 peptibody may be optimized for expression in a CHO cell.

A nucleic acid sequence encoding a GLP-2 peptibody as described in the present application, can be molecularly cloned (inserted) into a suitable vector for propagation or expression in a host cell. For example, the GLP-2 peptibody sequences comprising a signal peptide effective to secrete the GLP-2 peptibody from the host cell are inserted into the suitable vector, such as sequences selected from SEQ ID NOS: 2, 5, 8, 11, 14, 17, 20, 23, 26, 29 and 31. A wide variety of expression vectors can be used to practice the present invention, including, without limitation, a prokaryotic expression vector; a yeast expression vector; an insect expression vector and a mammalian expression vector. Exemplary vectors suitable for the present invention include, but are not limited to, viral based vectors (e.g., AAV based vectors, retrovirus based vectors, plasmid based vectors). In some embodiments, a nucleic acid sequence encoding a GLP-2 peptibody can be inserted into a suitable vector. In some embodiments, a nucleic acid sequence encoding a GLP-2 peptibody can be inserted into a suitable vector. Typically, a nucleic acid encoding a GLP-2 peptibody is operably linked to various regulatory sequences or elements.

Various regulatory sequences or elements may be incorporated in an expression vector suitable for the present invention. Exemplary regulatory sequences or elements include, but are not limited to, promoters, enhancers, repressors or suppressors, 5′ untranslated (or non-coding) sequences, introns, 3′ untranslated (or non-coding) sequences.

As used herein, a “promoter” or “promoter sequence” is a DNA regulatory region capable of binding an RNA polymerase in a cell (e.g., directly or through other promoter bound proteins or substances) and initiating transcription of a coding sequence. A promoter sequence is, in general, bound at its 3′ terminus by the transcription initiation site and extends upstream (5′ direction) to include the minimum number of bases or elements necessary to initiate transcription at any level. The promoter may be operably associated with or operably linked to the expression control sequences, including enhancer and repressor sequences or with a nucleic acid to be expressed. In some embodiments, the promoter may be inducible. In some embodiments, the inducible promoter may be unidirectional or bi-directional. In some embodiments, the promoter may be a constitutive promoter. In some embodiments, the promoter can be a hybrid promoter, in which the sequence containing the transcriptional regulatory region is obtained from one source and the sequence containing the transcription initiation region is obtained from a second source. Systems for linking control elements to coding sequence within a transgene are well known in the art (general molecular biological and recombinant DNA techniques are described in Sambrook, Fritsch, and Maniatis, Molecular Cloning: A Laboratory Manual, Second Edition, Cold Spring Harbor Laboratory Press, Cold Spring Harbor, N. Y., 1989, which is incorporated herein by reference). Commercial vectors suitable for inserting a transgene for expression in various host cells under a variety of growth and induction conditions are also well known in the art.

In some embodiments, a specific promoter may be used to control expression of the transgene in a mammalian host cell such as, but are not limited to, SRa-promoter (Takebe et al., Molec. and Cell. Bio. 8:466-472 (1988)), the human CMV immediate early promoter (Boshart et al., Cell 41:521-530 (1985); Foecking et al., Gene 45:101-105 (1986)), human CMV promoter, the human CMVS promoter, the murine CMV immediate early promoter, the EF1-α-promoter, a hybrid CMV promoter for liver specific expression (e.g., made by conjugating CMV immediate early promoter with the transcriptional promoter elements of either human α-1-antitrypsin (HAT) or albumin (HAL) promoter), or promoters for hepatoma specific expression (e.g., wherein the transcriptional promoter elements of either human albumin (HAL; about 1000 bp) or human α-1-antitrypsin (HAT, about 2000 bp) are combined with a 145 long enhancer element of human α-1-microglobulin and bikunin precursor gene (AMBP); HAL-AMBP and HAT-AMBP); the SV40 early promoter region (Benoist at al., Nature 290:304-310 (1981)), the Orgyia pseudotsugata immediate early promoter, the herpes thymidine kinase promoter (Wagner at al., Proc. Natl. Acad. Sci. USA 78:1441-1445 (1981)); or the regulatory sequences of the metallothionein gene (Brinster et al., Nature 296:39-42 (1982)). In some embodiments, the mammalian promoter is a is a constitutive promoter such as, but not limited to, the hypoxanthine phosphoribosyl transferase (HPTR) promoter, the adenosine deaminase promoter, the pyruvate kinase promoter, the beta-actin promoter as well as other constitutive promoters known to those of ordinary skill in the art.

In some embodiments, a specific promoter may be used to control expression of a transgene in a prokaryotic host cell such as, but are not limited to, the β-lactamase promoter (Villa-Komaroff et al., Proc. Natl. Acad. Sci. USA 75:3727-3731 (1978)); the tac promoter (DeBoer et al., Proc. Natl. Acad. Sci. USA 80:21-25 (1983)); the T7 promoter, the T3 promoter, the M13 promoter or the M16 promoter; in a yeast host cell such as, but are not limited to, the GAL1, GAL4 or GAL10 promoter, the ADH (alcohol dehydrogenase) promoter, PGK (phosphoglycerol kinase) promoter, alkaline phosphatase promoter, glyceraldehyde-3-phosphate dehydrogenase III (TDH3) promoter, glyceraldehyde-3-phosphate dehydrogenase II (TDH2) promoter, glyceraldehyde-3-phosphate dehydrogenase I (TDH1) promoter, pyruvate kinase (PYK), enolase (ENO), or triose phosphate isomerase (TPI).

In some embodiments, the promoter may be a viral promoter, many of which are able to regulate expression of a transgene in several host cell types, including mammalian cells. Viral promoters that have been shown to drive constitutive expression of coding sequences in eukaryotic cells include, for example, simian virus promoters, herpes simplex virus promoters, papilloma virus promoters, adenovirus promoters, human immunodeficiency virus (HIV) promoters, Rous sarcoma virus promoters, cytomegalovirus (CMV) promoters, the long terminal repeats (LTRs) of Moloney murine leukemia virus and other retroviruses, the thymidine kinase promoter of herpes simplex virus as well as other viral promoters known to those of ordinary skill in the art.

In some embodiments, the gene control elements of an expression vector may also include 5′ non-transcribing and 5′ non-translating sequences involved with the initiation of transcription and translation, respectively, such as a TATA box, capping sequence, CAAT sequence, Kozak sequence and the like. Enhancer elements can optionally be used to increase expression levels of a polypeptide or protein to be expressed. Examples of enhancer elements that have been shown to function in mammalian cells include the SV40 early gene enhancer, as described in Dijkema et al., EMBO J. (1985) 4: 761 and the enhancer/promoter derived from the long terminal repeat (LTR) of the Rous Sarcoma Virus (RSV), as described in Gorman et al., Proc. Natl. Acad. Sci. USA (1982b) 79:6777 and human cytomegalovirus, as described in Boshart et al., Cell (1985) 41:521. Genetic control elements of an expression vector will also include 3′ non-transcribing and 3′ non-translating sequences involved with the termination of transcription and translation. Respectively, such as a poly polyadenylation (polyA) signal for stabilization and processing of the 3′ end of an mRNA transcribed from the promoter. Exemplary polyA signals include, for example, the rabbit beta globin polyA signal, bovine growth hormone polyA signal, chicken beta globin terminator/polyA signal, and SV40 late polyA region.

Expression vectors will preferably but optionally include at least one selectable marker. In some embodiments, the selectable maker is a nucleic acid sequence encoding a resistance gene operably linked to one or more genetic regulatory elements, to bestow upon the host cell the ability to maintain viability when grown in the presence of a cytotoxic chemical and/or drug. In some embodiments, a selectable agent may be used to maintain retention of the expression vector within the host cell. In some embodiments, the selectable agent is may be used to prevent modification (i.e. methylation) and/or silencing of the transgene sequence within the expression vector. In some embodiments, a selectable agent is used to maintain episomal expression of the vector within the host cell. In some embodiments, the selectable agent is used to promote stable integration of the transgene sequence into the host cell genome. In some embodiments, an agent and/or resistance gene may include, but is not limited to, methotrexate (MTX), dihydrofolate reductase (DHFR, U.S. Pat. Nos. 4,399,216; 4,634,665; 4,656,134; 4,956,288; 5,149,636; 5,179,017, ampicillin, neomycin (G418), zeomycin, mycophenolic acid, or glutamine synthetase (GS, U.S. Pat. Nos. 5,122,464; 5,770,359; 5,827,739) for eukaryotic host cell; tetracycline, ampicillin, kanamycin or chlorampenichol for a prokaryotic host cell; and URA3, LEU2, HIS3, LYS2, HIS4, ADE8, CUP1 or TRP1 for a yeast host cell.

Expression vectors may be transfected, transformed or transduced into a host cell. As used herein, the terms “transfection,” “transformation” and “transduction” all refer to the introduction of an exogenous nucleic acid sequence into a host cell. In some embodiments, expression vectors containing nucleic acid sequences encoding for a GLP-2 peptibody are transfected, transformed or transduced into a host cell at the same time. In some embodiments, expression vectors containing nucleic acid sequences encoding for a GLP-2 peptibody are transfected, transformed or transduced into a host cell sequentially.

Examples of transformation, transfection and transduction methods, which are well known in the art, include liposome delivery, i.e., Lipofectamine™ (Gibco BRL) Method of Hawley-Nelson, Focus 15:73 (1193), electroporation, CaPO₄ delivery method of Graham and van der Erb, Virology, 52:456-457 (1978), DEAE-Dextran medicated delivery, microinjection, biolistic particle delivery, polybrene mediated delivery, cationic mediated lipid delivery, transduction, and viral infection, such as, e.g., retrovirus, lentivirus, adenovirus adeno-associated virus and Baculovirus (Insect cells).

Once introduced inside cells, expression vectors may be integrated stably in the genome or exist as extra-chromosomal constructs. Vectors may also be amplified and multiple copies may exist or be integrated in the genome. In some embodiments, cells of the invention may contain 1, 2, 3, 4, 5, 6, 7, 8, 9, 10, 15, 20 or more copies of nucleic acids encoding a GLP-2 peptibody. In some embodiments, cells of the invention may contain 1, 2, 3, 4, 5, 6, 7, 8, 9, 10, 15, 20 or more copies of nucleic acids encoding a GLP-2 peptibody.

### Host Cells

In another aspect is provided a host cell comprising the polynucleotides described herein, e.g., those that allow for expression of a GLP-2 peptibody in the host cell. The host cell may be a Chinese hamster ovary cell. Alternatively, the host cell can be a mammalian cell, with non-limiting examples including a BALB/c mouse myeloma line (NSO/1, ECACC No: 85110503); human retinoblasts (PER.C6, CruCell, Leiden, The Netherlands); a monkey kidney CV1 line transformed by SV40 (COS-7, ATCC CRL 1651); a human embryonic kidney line (HEK293 or 293 cells subcloned for growth in suspension culture, Graham et al., J. Gen Virol., 36:59, 1977); a human fibrosarcoma cell line (e.g., HT1080); baby hamster kidney cells (BHK21, ATCC CCL 10); Chinese hamster ovary cells (CHO, Urlaub and Chasin, Proc. Natl. Acad. Sci. USA, 77:4216, 1980), including CHO EBNA (Daramola O. et al., Biotechnol. Prog., 2014, 30(1):132-41) and CHO GS (Fan L. et al., Biotechnol. Bioeng. 2012, 109(4):1007-15; mouse sertoli cells (TM4, Mather, Biol. Reprod., 23:243-251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TM cells (Mather et al., Annals N.Y. Acad. Sci., 383:44-68, 1982); MRC 5 cells; FS4 cells; and a human hepatoma line (Hep G2).

The polynucleotide may in an expression plasmid. The expression plasmid may have any number of origins of replication known to those of ordinary skill in the art. The polynucleotide or expression plasmid may be introduced into the host cell by any number of ways known to those of ordinary skill in the art. For example, a flow electroporation system, such as the MaxCyte GT®, MaxCyte VLX®, or MaxCyte STX® transfection systems, can be used to introduce the polynucleotide or expression plasmid into the host cell.

In various embodiments, the host cell expresses the polynucleotide. The host cell may express GLP-2 peptibody at a level sufficient for fed-batch cell culture scale or other large scale. Alternative methods to produce recombinant GLP-2 peptibodies at a large scale include roller bottle cultures and bioreactor batch cultures. In some embodiments, recombinant GLP-2 peptibody protein is produced by cells cultured in suspense. In some embodiments, recombinant GLP-2 peptibody protein is produced by adherent cells.

### Production

A recombinant GLP-2 peptibody may be produced by any available means. For example, a recombinant GLP-2 peptibody may be recombinantly produced by utilizing a host cell system engineered to express a recombinant GLP-2 peptibody-encoding nucleic acid. Alternatively or additionally, a recombinant GLP-2 peptibody may be produced by activating endogenous genes. Alternatively or additionally, a recombinant GLP-2 peptibody may be partially or fully prepared by chemical synthesis. Alternatively, a recombinant GLP-2 peptibody can be produced in vivo by mRNA therapeutics.

In some embodiments, recombinant GLP-2 peptibodies are produced in mammalian cells. Non-limiting examples of mammalian cells that may be used in accordance with the present invention include BALB/c mouse myeloma line (NSO/1, ECACC No: 85110503); human retinoblasts (PER.C6, CruCell, Leiden, The Netherlands); monkey kidney CV1 line transformed by SV40 (COS-7, ATCC CRL 1651); human embryonic kidney line (HEK293 or 293 cells subcloned for growth in suspension culture, Graham et al., J. Gen Virol., 36:59, 1977); human fibrosarcoma cell line (e.g., HT1080); baby hamster kidney cells (BHK21, ATCC CCL 10); Chinese hamster ovary cells +/−DHFR (CHO, Urlaub and Chasin, Proc. Natl. Acad. Sci. USA, 77:4216, 1980), including CHO EBNA (Daramola O. et al., Biotechnol. Prog., 2014, 30(1):132-41) and CHO GS (Fan L. et al., Biotechnol. Bioeng. 2012, 109(4):1007-15; mouse sertoli cells (TM4, Mather, Biol. Reprod., 23:243-251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TM cells (Mather et al., Annals N.Y. Acad. Sci., 383:44-68, 1982); MRC 5 cells; FS4 cells; and a human hepatoma line (Hep G2).

In some embodiments, recombinant GLP-2 peptibodies are produced from human cells. In some embodiments, recombinant GLP-2 peptibodies are produced from CHO cells or HT1080 cells.

In certain embodiments, a host cell is selected for generating a cell line based on certain preferable attributes or growth under particular conditions chosen for culturing cells. It will be appreciated by one skilled in the art, such attributes may be ascertained based on known characteristic and/or traits of an established line (i.e. a characterized commercially available cell line) or though empirical evaluation. In some embodiments, a cell line may be selected for its ability to grow on a feeder layer of cells. In some embodiments, a cell line may be selected for its ability to grow in suspension. In some embodiments, a cell line may be selected for its ability to grow as an adherent monolayer of cells. In some embodiments, such cells can be used with any tissue culture vessel or any vessel treated with a suitable adhesion substrate. In some embodiments, a suitable adhesion substrate is selected from the group consisting of collagen (e.g. collagen I, II, II, or IV), gelatin, fibronectin, laminin, vitronectin, fibrinogen, BD Matrigel™, basement membrane matrix, dermatan sulfate proteoglycan, Poly-D-Lysine and/or combinations thereof. In some embodiments, an adherent host cell may be selected and modified under specific growth conditions to grow in suspension. Such methods of modifying an adherent cell to grown in suspension are known in the art. For example, a cell may be conditioned to grow in suspension culture, by gradually removing animal serum from the growth media over time.

Typically, cells that are engineered to express a recombinant GLP-2 peptibody may comprise a transgene that encodes a recombinant GLP-2 peptibody described herein. It should be appreciated that the nucleic acids encoding recombinant GLP-2 peptibodies may contain regulatory sequences, gene control sequences, promoters, non-coding sequences and/or other appropriate sequences for expressing the recombinant GLP-2 peptibody. Typically, the coding region is operably linked with one or more of these nucleic acid components.

In some embodiments, a recombinant GLP-2 peptibody is produced in vivo by mRNA therapeutics. An mRNA encoding for a GLP-2 peptibody is prepared and administered to a patient in need of the GLP-2 peptibody. The mRNA can comprise a sequence corresponding to the DNA sequences of SEQ ID NOS: 3, 6, 9, 12, 15, 18, 21, 24, 27 and 30. Various routes of administration may be used, such as injection, nebulization in the lungs, and electroporation under the skin. The mRNA may be encapsulated in a viral vector or a nonviral vector. Exemplary nonviral vectors include liposomes, cationic polymers and cubosomes.

### Recovery and Purification

Various means for purifying the GLP-2 peptibodies from the cells may be used. Various methods may be used to purify or isolate GLP-2 peptibodies produced according to various methods described herein. In some embodiments, the expressed enzyme is secreted into the medium and thus cells and other solids may be removed, as by centrifugation or filtering for example, as a first step in the purification process. Alternatively or additionally, the expressed enzyme is bound to the surface of the host cell. In this embodiment, the host cells expressing the polypeptide or protein are lysed for purification. Lysis of mammalian host cells can be achieved by any number of means well known to those of ordinary skill in the art, including physical disruption by glass beads and exposure to high pH conditions.

The GLP-2 peptibodies may be isolated and purified by standard methods including, but not limited to, chromatography (e.g., ion exchange, affinity, size exclusion, and hydroxyapatite chromatography), gel filtration, centrifugation, or differential solubility, ethanol precipitation or by any other available technique for the purification of proteins. See, e.g., Scopes, Protein Purification Principles and Practice 2nd Edition, Springer-Verlag, New York, 1987; Higgins, S. J. and Hames, B. D. (eds.), Protein Expression: A Practical Approach, Oxford Univ Press, 1999; and Deutscher, M. P., Simon, M. I., Abelson, J. N. (eds.), Guide to Protein Purification: Methods in Enzymology (Methods in Enzymology Series, Vol 182), Academic Press, 1997, all incorporated herein by reference. For immunoaffinity chromatography in particular, the protein may be isolated by binding it to an affinity column comprising antibodies that were raised against that protein and were affixed to a stationary support. Alternatively, affinity tags such as an influenza coat sequence, poly-histidine, or glutathione-S-transferase can be attached to the protein by standard recombinant techniques to allow for easy purification by passage over the appropriate affinity column. Protease inhibitors such as phenyl methyl sulfonyl fluoride (PMSF), leupeptin, pepstatin or aprotinin may be added at any or all stages in order to reduce or eliminate degradation of the polypeptide or protein during the purification process. Protease inhibitors are particularly desired when cells must be lysed in order to isolate and purify the expressed polypeptide or protein.

A GLP-2 peptibody or specified portion or variant can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, mixed mode chromatography (e.g., MEP Hypercel™), hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, N.Y. (1997-2003).

Peptibodies or specified portions or variants of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the GLP-2 peptibody or specified portion or variant of the present invention can be glycosylated or can be non-glycosylated, with glycosylated preferred.

### Formulations

In some embodiments, the pharmaceutical compositions described herein further comprise a carrier. Suitable acceptable carriers include but are not limited to water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents (e.g., diluents, buffers, lipophilic solvents, preservatives, adjuvants, lubricants, preservatives, stabilizers, wetting agents, emulsifiers, salts for influencing osmotic pressure, buffers, coloring, flavoring and/or aromatic substances and the like) which do not deleteriously react with the active compounds or interference with their activity. In some embodiments, a water-soluble carrier suitable for intravenous administration is used.

Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18^{th} Edition, Mack Publishing Co. (Easton, Pa.) 1990. Pharmaceutically acceptable carriers can be routinely selected that are suitable for the mode of administration, solubility and/or stability of the GLP-2 peptibody composition as well known in the art or as described herein. For example, sterile saline and phosphate-buffered saline at slightly acidic or physiological pH may be used. pH buffering agents may be phosphate, citrate, acetate, tris/hydroxymethyl)aminomethane (TRIS), N-Tris(hydroxymethyl)methyl-3-aminopropanesulphonic acid (TAPS), ammonium bicarbonate, diethanolamine, histidine, which is a preferred buffer, arginine, lysine, or acetate or mixtures thereof. Preferred buffer ranges are pH 4-8, pH 6.5-8, more preferably pH 7-7.5. Preservatives, such as para, meta, and ortho-cresol, methyl- and propylparaben, phenol, benzyl alcohol, sodium benzoate, benzoic acid, benzyl-benzoate, sorbic acid, propanoic acid, esters of p-hydroxybenzoic acid may be provided in the pharmaceutical composition. Stabilizers, preventing oxidation, deamidation, isomerisation, racemisation, cyclisation, peptide hydrolysis, such as, e.g., ascorbic acid, methionine, tryptophane, EDTA, asparagine, lysine, arginine, glutamine and glycine may be provided in the pharmaceutical composition. Stabilizers, preventing aggregation, fibrillation, and precipitation, such as sodium dodecyl sulfate, polyethylene glycol, carboxymethyl cellulose, cyclodextrine may be provided in the pharmaceutical composition. Organic modifiers for solubilization or preventing aggregation, such as ethanol, acetic acid or acetate and salts thereof may be provided in the pharmaceutical composition. Isotonicity makers, such as salts, e.g., sodium chloride or most preferred carbohydrates, e.g., dextrose, mannitol, lactose, trehalose, sucrose or mixtures thereof may be provided in the pharmaceutical composition.

Pharmaceutical excipients and additives useful in the present composition include but are not limited to proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/GLP-2 peptibody or specified portion or variant components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients may be used, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like.

GLP-2 peptibody compositions can also include a buffer or a pH adjusting agent; typically, the buffer is a salt prepared from an organic acid or base. Exemplary buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers.

Additionally, the GLP-2 peptibody or specified portion or variant compositions of the invention can include polymeric excipients/additives such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the GLP-2 peptibody compositions according to the invention are known in the art, e.g., as listed in “Remington: The Science & Practice of Pharmacy”, 21^{st} ed., Williams & Williams, (2005), and in the “Physician's Desk Reference”, 71^{st} ed., Medical Economics, Montvale, N.J. (2017), the disclosures of which are entirely incorporated herein by reference. Preferred carrier or excipient materials are carbohydrates (e.g., saccharides and alditols) and buffers (e.g., citrate) or polymeric agents.

The pharmaceutical compositions may be formulated as a liquid suitable for administration by intravenous or subcutaneous injection or infusion. The liquid may comprise one or more solvents. Exemplary solvents include, but are not limited to water; alcohols such as ethanol and isopropyl alcohol; vegetable oil; polyethylene glycol; propylene glycol; and glycerin or mixing and combination thereof. A water-soluble carrier suitable for intravenous administration may be used. For example, in some embodiments, a composition for intravenous administration typically is a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

As noted above, formulations can preferably include a suitable buffer with saline or a chosen salt, as well as optional preserved solutions and formulations containing a preservative as well as multi-use preserved formulations suitable for pharmaceutical or veterinary use, comprising at least one GLP-2 peptibody or specified portion or variant in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4., 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3, 0.4, 0.5, 0.9, 1.0%), 0.1-3% benzyl alcohol (e.g., 0.5, 0.9, 1.1., 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

The GLP-2 peptibodies may be formulated for parenteral administration and can contain as common excipients sterile water or saline, polyalkylene glycols such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent such as aqueous solution or a sterile injectable solution or suspension in a solvent. As the usable vehicle or solvent, water, Ringer's solution, isotonic saline, etc. are allowed; as an ordinary solvent, or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446.

The pharmaceutical compositions may be an extended release formulation. The pharmaceutical compositions may also be formulated for sustained release, extended release, delayed release or slow release of the GLP-2 peptibody, e.g., comprising the amino acid sequence of SEQ ID NO: 1 or SEQ ID NO: 7. Extended release, also known as controlled release and sustained release, can be provided to injectable formulations. Microspheres, nanospheres, implants, depots, and polymers may be used in combination with any of the compounds, methods, and formulations described herein to provide an extended release profile.

The GLP-2 peptibody, e.g., comprising the amino acid sequence of SEQ ID NO: 1 or SEQ ID NO: 7, may be formulated in a concentration of 10 to 100 mg/mL. The concentration may be about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 28 mg/mL, about 30 mg/mL, about 32 mg/mL, about 34 mg/mL, about 36 mg/mL, about 38 mg/mL, about 40 mg/mL, about 42 mg/mL, about 44 mg/mL, about 46 mg/mL, about 48 mg/mL, about 50 mg/mL, about 55 mg/mL, about 60 mg/mL, about 65 mg/mL, about 70 mg/mL, about 75 mg/mL, about 80 mg/mL, about 85 mg/mL, about 90 mg/mL, about 95 mg/mL, about 99 mg/mL, with “about” meaning from 0.5 mg/mL below to 0.5 mg/mL above the referred to value. The concentration may be from 10 to 15 mg/mL, 11 to 16 mg/mL, 12 to 17 mg/mL, 13 to 18 mg/mL, 14 to 19 mg/mL, 15 to 20 mg/mL, 16 to 21 mg/mL, 17 to 22 mg/mL, 18 to 23 mg/mL, 19 to 24 mg/mL, 20 to 25 mg/mL, 25 to 30 mg/mL, 30 to 35 mg/mL, 35 to 40 mg/mL, 40 to 45 mg/mL, 45 to 50 mg/mL, 50 to 55 mg/mL, 55 to 60 mg/mL, 60 to 65 mg/mL, 65 to 70 mg/mL, 70 to 75 mg/mL, 75 to 80 mg/mL, 80 to 85 mg/mL, 85 to 90 mg/mL, or 90 to 100 mg/mL. The concentration may be from 12 to 18 mg/mL, 13 to 17 mg/mL, 14 to 16 mg/mL or from 14.5 to 15.5 mg/mL, or 15 mg/mL.

Formulations and compositions comprising the GLP-2 peptibody can optionally further comprise an effective amount of at least one compound or protein selected from at least one of a diabetes or insulin metabolism related drug, an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplactic, an immunomodulation drug, an ophthalmic, otic or nasal drug, a topical drug, a nutritional drug or the like. Such drugs are well known in the art, including formulations, indications, dosing and administration for each presented herein (see e.g., Nursing 2001 Handbook of Drugs, 21^{st} edition, Springhouse Corp., Springhouse, Pa., 2001; Health Professional's Drug Guide 2001, ed., Shannon, Wilson, Stang, Prentice-Hall, Inc, Upper Saddle River, N.J.; Pharmacotherapy Handbook, Wells et al., ed., Appleton & Lange, Stamford, Conn., each entirely incorporated herein by reference).

GLP-2 peptibodies may also be formulated as a slow release implantation device for extended or sustained administration of the GLP-2 peptibody. Such sustained release formulations may be in the form of a patch positioned externally on the body. Examples of sustained release formulations include composites of biocompatible polymers, such as poly(lactic acid), poly(lactic-co-glycolic acid), methylcellulose, hyaluronic acid, sialic acid, silicate, collagen, liposomes and the like. Sustained release formulations may be of particular interest when it is desirable to provide a high local concentration of a GLP-2 peptibody.

GLP-2 peptibody compositions and formulations can be provided to patients as clear solutions or as dual vials comprising a vial of lyophilized at least one GLP-2 peptibody (e.g., comprising the amino acid sequence of SEQ ID NO: 1 or SEQ ID NO: 7) or specified portion or variant that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available.

GLP-2 peptibody compositions and formulations can be provided indirectly to patients by providing to pharmacies, clinics, or other such institutions and facilities, clear solutions or dual vials comprising a vial of lyophilized at least one GLP-2 peptibody (e.g., comprising the amino acid sequence of SEQ ID NO: 1 or SEQ ID NO: 7) or specified portion or variant that is reconstituted with a second vial containing the aqueous diluent. The clear solution in this case can be up to one liter or even larger in size, providing a large reservoir from which smaller portions of a GLP-2 peptibody (e.g., comprising the amino acid sequence of SEQ ID NO: 1 or SEQ ID NO: 7) or specified portion or variant solution can be retrieved one or multiple times for transfer into smaller vials and provided by the pharmacy or clinic to their customers and/or patients. Such products can include packaging material. The packaging material can provide, in addition to the information required by the regulatory agencies, the conditions under which the product can be used. The packaging material can provide instructions to the patient to reconstitute a GLP-2 peptibody (e.g., comprising the amino acid sequence of SEQ ID NO: 1 or SEQ ID NO: 7) or specified portion or variant in the aqueous diluent to form a solution and to use the solution over a period of 2-24 hours or greater for the two vial, wet/dry product.

### Treatment

In another aspect is provided a method for treating a patient with enterocutaneous fistula (ECF) comprising treating the patient with a GLP-2 peptibody comprising the amino acid sequence of SEQ ID NO: 1 or SEQ ID NO: 7 using a dosing regimen effective to promote closure, healing, and/or repair of the ECF. The GLP-2 peptibodies may be particularly effective to treat ECF because they have a longer half-life than GLP-2 or teduglutide. The longer half-life provides for less frequent dosing and a lower peak-to-trough ratio.

High mortality and morbidity arise from ECF. Further, ECF can occur from having an intra-abdominal procedure. Damage to the bowel wall carries the greatest risk of an ECF. See Galie, K. L. et al., “Postoperative Enterocutaneous Fistula: When to Reoperate and How to Succeed” Clin. Colon Rectal Surg., 2006, 19:237-246; Arebi, N. et al., “High-Output Fistula” Clinics in Colon and Rectal Surgery, 2004, 17(2):89-98. Without wishing to be bound by theory, ECF is an opening between the gastrointestinal tract and the skin. Substantial amounts of fluid, nutrients, and gastrointestinal fluid can leave the gastrointestinal tract without adequate absorption by the small intestine. Reduction of gastric secretions and improvement of absorption of nutrients can improve the prognosis of ECF.

In some embodiments, the method is effective to enhance intestinal absorption by the patient. In some embodiments, the method is effective to enhance intestinal absorption of nutrients, e.g., polypeptides, carbohydrates, fatty acids, vitamins, minerals, and water. In some embodiments, the method is effective to reduce the volume of gastric secretions in the patient. The GLP-2 peptibody may be effective to reduce the amount of gastrointestinal secretions that reach the skin, such as by migrating through the fistula. Activation of the GLP-2 for a longer period of time could reduce gastric secretion and output of fluid through the fistula, thereby more quickly promoting recovery and allowing the fistula to heal more quickly. Also, increased collagen expression and decreased metalloprotease expression has been observed after teduglutide treatment. See Costa, B. P. et al., “Teduglutide effects on gene regulation of fibrogenesis on an animal model of intestinal anastomosis” Journal of Surgical Research, August 2017 (216); 87-98. In some embodiments, the method is effective to increase villus height in the small intestine of the patient. In some embodiments, the method is effective to increase the crypt depth in the small intestine of the patient.

The GLP-2 peptibody, e.g., comprising the amino acid sequence of SEQ ID NO: 1 or SEQ ID NO: 7, may be administered subcutaneously or intravenously. In various embodiments, multiple administrations are performed according to a dosing regimen. As used herein, the term “Q2D” means administration every two days, “Q3D” means administration every three days, etc. “QW” means administration every week. “BID” means administration twice a day. Dosing can be undertaken BID, once per day (QD), Q2D, Q3D, Q4D, Q5D, Q6D, QW, once every 8 days, once every 9 days, once every 10 days, once every 11 days, once every 12 days, once every 13 days, once every two weeks, once every 15 days, once every 16 days, or once every 17 days, once every three weeks, or once every month, for example. The GLP-2 peptibody (e.g., comprising the amino acid sequence of SEQ ID NO: 1 or SEQ ID NO: 7) may be administered subcutaneously according to a dosage regimen of between 0.02 to 3.0 mg/kg, 0.02 to 0.5 mg/kg, 0.04 to 0.45 mg/kg, 0.08 to 0.4 mg/kg, 0.10 to 0.35 mg/kg, 0.20 to 0.30 mg/kg, 0.02 to 0.05 mg/kg, 0.03 to 0.04 mg/kg, 0.05 to 0.10 mg/kg, 0.10 to 0.15 mg/kg, 0.2 to 0.3 mg/kg, 0.3 to 0.4 mg/kg, 0.4 to 0.5 mg/kg, 0.5 to 0.8 mg/kg, 0.7 to 1.0 mg/kg, 0.9 to 1.2 mg/kg, 1.0 to 1.5 mg/kg, 1.2 to 1.8 mg/kg, 1.5 to 2.0 mg/kg, 1.7 to 2.5 mg/kg, or 2.0 to 3.0 mg/kg, once every 2-14 days, every 5-8 days, or every week (QW). The GLP-2 peptibody (e.g., comprising the amino acid sequence of SEQ ID NO: 7) may be administered subcutaneously according to a dosage regimen of between 0.2 to 1.4 mg/kg, 0.3 to 1.0 mg/kg, 0.4 to 0.9 mg/kg, 0.5 to 0.8 mg/kg, 0.3 to 0.7 mg/kg, 0.6 to 1.0 mg/kg, 0.2 to 0.4 mg/kg, 0.3 to 0.5 mg/kg, 0.4 to 0.6 mg/kg, 0.5 to 0.7 mg/kg, 0.6 to 0.8 mg/kg, 0.7 to 0.9 mg/kg, 0.8 to 1.0 mg/kg, 0.9 to 1.1 mg/kg, 1.0 to 1.2 mg/kg, 1.1 to 1.3 mg/kg, and 1.2 to 1.4 mg/kg, every week (QW) or every two weeks.

Alternatively, the GLP-2 peptibody could be administered every three weeks or once a month, such as for maintenance purposes. The GLP-2 peptibody (e.g., comprising the amino acid sequence of SEQ ID NO: 7) may be administered subcutaneously according to a dosage regimen of between 0.2 to 1.4 mg/kg, 0.3 to 1.0 mg/kg, 0.4 to 0.9 mg/kg, 0.5 to 0.8 mg/kg, 0.3 to 0.7 mg/kg, 0.6 to 1.0 mg/kg, 0.2 to 0.4 mg/kg, 0.3 to 0.5 mg/kg, 0.4 to 0.6 mg/kg, 0.5 to 0.7 mg/kg, 0.6 to 0.8 mg/kg, 0.7 to 0.9 mg/kg, 0.8 to 1.0 mg/kg, 0.9 to 1.1 mg/kg, 1.0 to 1.2 mg/kg, 1.1 to 1.3 mg/kg, and 1.2 to 1.4 mg/kg, every three weeks or once a month.

As an alternative, GLP-2 peptibody (e.g., comprising the amino acid sequence of SEQ ID NO: 1 or SEQ ID NO: 7) may be administered subcutaneously according to a dosage regimen of between 0.02 to 0.5 mg/kg, 0.04 to 0.45 mg/kg, 0.08 to 0.4 mg/kg, 0.10 to 0.35 mg/kg, 0.20 to 0.30 mg/kg every 5-8 days, or every week (QW) for maintenance purposes. The GLP-2 peptibody comprising the amino acid sequence of SEQ ID NO: 1 or SEQ ID NO: 7 may be administered in a concentration of 10 to 100 mg/mL, 10 to 90 mg/mL, 20 to 80 mg/mL, 25 to 75 mg/mL, 30 to 70 mg/mL, 50 to 100 mg/mL, 60 to 90 mg/mL, about 75 mg/mL, 75 mg/mL, 10 to 20 mg/mL, 15 to 25 mg/mL, 12 to 18 mg/mL, 13-17 mg/mL, 14-16 mg/mL, about 15 mg/mL or 15 mg/mL.

The above dosing regimens may be conducted over six months to one year to treat ECF. GLP-2 peptibodies can be administered once a month after the initial dosage regimen for maintenance and to prevent relapse.

As used herein, the term “subcutaneous tissue”, is defined as a layer of loose, irregular connective tissue immediately beneath the skin. For example, the subcutaneous administration may be performed by injecting a composition into areas including, but not limited to, the thigh region, abdominal region, gluteal region, or scapular region. For such purposes, the formulation may be injected using a syringe. However, other devices for administration of the formulation are available such as injection devices (e.g., the Inject-ease™ and Genject™ devices); injector pens (such as the GenPen™); needleless devices (e.g., MediJector™ and BioJector™); and subcutaneous patch delivery systems. In some embodiments, a GLP-2 peptibody, e.g., comprising the amino acid sequence of SEQ ID NO: 1 or SEQ ID NO: 7, or a pharmaceutical composition containing the same is administered intravenously.

In various embodiments, the above methods of treating ECF are used in conjunction with known methods treat ECF. Exemplary known methods include parenteral nutrition, antibiotic administration to prevent sepsis, ostomy appliances attached to exterior opening of the fistula, sump drains, fistuloclysis, vitamin supplementation, mineral supplementation, use of H2 blockers or proton pump inhibitors to suppress acid, administration of histoacryl glue and administration of fibrin glue.

In another aspect is provided a method for treating a patient with obstructive jaundice comprising treating the patient with a GLP-2 peptibody, e.g., comprising the amino acid sequence of SEQ ID NO: 1 or SEQ ID NO: 7, using a dosing regimen effective to treat the obstructive jaundice. Obstructive jaundice occurs when the flow of bile to the intestine is blocked and remains in the bloodstream. Gallstones can cause obstructive jaundice. Intestinal barrier function may be damaged or reduced in patients with obstructive jaundice, which can result in bacterial translocation across the small intestine. GLP-2 peptibodies described herein may prevent damage to intestinal barrier function during an episode of obstructive jaundice.

A dosing regimen may be used that is effective to treat the obstructive jaundice. The GLP-2 peptibody, e.g., comprising the amino acid sequence of SEQ ID NO: 1 or SEQ ID NO: 7, may be administered subcutaneously or intravenously. In various embodiments, multiple administrations are performed according to a dosing regimen. As used herein, the term “Q2D” means administration every two days, “Q3D” means administration every three days, etc. “QW” means administration every week. “BID” means administration twice a day. Dosing can be undertaken BID, once per day (QD), Q2D, Q3D, Q4D, Q5D, Q6D, QW, once every 8 days, once every 9 days, once every 10 days, once every 11 days, once every 12 days, once every 13 days, once every two weeks, once every 15 days, once every 16 days, or once every 17 days, once every three weeks, or once every month, for example. The GLP-2 peptibody (e.g., comprising the amino acid sequence of SEQ ID NO: 1 or SEQ ID NO: 7) may be administered subcutaneously according to a dosage regimen of between 0.02 to 3.0 mg/kg, 0.02 to 0.5 mg/kg, 0.04 to 0.45 mg/kg, 0.08 to 0.4 mg/kg, 0.10 to 0.35 mg/kg, 0.20 to 0.30 mg/kg, 0.02 to 0.05 mg/kg, 0.03 to 0.04 mg/kg, 0.05 to 0.10 mg/kg, 0.10 to 0.15 mg/kg, 0.2 to 0.3 mg/kg, 0.3 to 0.4 mg/kg, 0.4 to 0.5 mg/kg, 0.5 to 0.8 mg/kg, 0.7 to 1.0 mg/kg, 0.9 to 1.2 mg/kg, 1.0 to 1.5 mg/kg, 1.2 to 1.8 mg/kg, 1.5 to 2.0 mg/kg, 1.7 to 2.5 mg/kg, or 2.0 to 3.0 mg/kg once every 2-14 days, every 5-8 days, or every week (QW). The GLP-2 peptibody (e.g., comprising the amino acid sequence of SEQ ID NO: 7) may be administered subcutaneously according to a dosage regimen of between 0.2 to 1.4 mg/kg, 0.3 to 1.0 mg/kg, 0.4 to 0.9 mg/kg, 0.5 to 0.8 mg/kg, 0.3 to 0.7 mg/kg, 0.6 to 1.0 mg/kg, 0.2 to 0.4 mg/kg, 0.3 to 0.5 mg/kg, 0.4 to 0.6 mg/kg, 0.5 to 0.7 mg/kg, 0.6 to 0.8 mg/kg, 0.7 to 0.9 mg/kg, 0.8 to 1.0 mg/kg, 0.9 to 1.1 mg/kg, 1.0 to 1.2 mg/kg, 1.1 to 1.3 mg/kg, and 1.2 to 1.4 mg/kg, every week (QW) or every two weeks.

Alternatively, the GLP-2 peptibody could be administered every three weeks or once a month, such as for maintenance purposes. The GLP-2 peptibody (e.g., comprising the amino acid sequence of SEQ ID NO: 1 or SEQ ID NO: 7) may be administered subcutaneously according to a dosage regimen of between 0.02 to 0.5 mg/kg, 0.04 to 0.45 mg/kg, 0.08 to 0.4 mg/kg, 0.10 to 0.35 mg/kg, 0.20 to 0.30 mg/kg every 5-8 days or every week (QW) for maintenance purposes. The GLP-2 peptibody (e.g., comprising the amino acid sequence of SEQ ID NO: 1 or SEQ ID NO: 7) may be administered in a concentration of 10 to 100 mg/mL, 10 to 90 mg/mL, 20 to 80 mg/mL, 25 to 75 mg/mL, 30 to 70 mg/mL, 50 to 100 mg/mL, 60 to 90 mg/mL, about 75 mg/mL, 75 mg/mL, 10 to 20 mg/mL, 15 to 25 mg/mL, 12 to 18 mg/mL, 13-17 mg/mL, 14-16 mg/mL, about 15 mg/mL or 15 mg/mL.

For example, the subcutaneous administration may be performed by injecting a composition into areas including, but not limited to, the thigh region, abdominal region, gluteal region, or scapular region. For such purposes, the formulation may be injected using a syringe. However, other devices for administration of the formulation are available such as injection devices (e.g., the Inject-ease™ and Genject™ devices); injector pens (such as the GenPen™); needleless devices (e.g., MediJector™ and BioJector™); and subcutaneous patch delivery systems. In some embodiments, a GLP-2 peptibody (e.g., comprising the amino acid sequence of SEQ ID NO: 1 or SEQ ID NO: 7), or a pharmaceutical composition containing the same is administered intravenously.

In some embodiments, the level of serum bilirubin is reduced as compared to the level of serum bilirubin before said treatment. Serum bilirubin reflects the extent of jaundice and is the source of the yellow color in skin and eyes seen in patients with obstructive jaundice. In some embodiments, the method is effective to enhance intestinal absorption in the patient. In some embodiments, the method is effective to enhance intestinal absorption of nutrients, e.g., polypeptides, carbohydrates, fatty acids, vitamins, minerals, and water. In some embodiments, the method is effective to increase villus height in small intestine of the patient. In some embodiments, the method is effective to increase crypt depth in small intestine of the patient. In some embodiments, the method is effective to increase crypt organization in small intestine of the patient. In some embodiments, the method is effective to improve intestinal barrier function in the patient and to reduce the rate of bacteria translocation across the small intestine of the patient.

In another aspect, the present invention provides a method for treating, ameliorating or protecting against radiation damage, and/or the effects thereof, to the gastrointestinal tract, comprising administering a GLP-2 peptibody that, for example, comprises the amino acid sequence of SEQ ID NO: 1 or SEQ ID NO: 7. A dosing regimen effective to treat or prevent radiation damage to the gastrointestinal tract of the patient may be used. The radiation damage may be in the small intestine. In some embodiments, the method is effective to reduce apoptosis in cells of the gastrointestinal tract.

Radiation damage to the small intestine may result in cell damage that is sufficient to cause one or more of the following effects: decreased intestinal barrier function, reduced absorption of water and other nutrients by the small intestine, increased dependency on parenteral nutrition. A GLP-2 peptibody having a substantially greater half-life than GLP-2 or teduglutide could reverse these effects. Without wishing to be bound by theory, GLP-2 may prevent cells in the small intestine from undergoing apoptosis by promoting Akt phosphorylation in such cells, e.g., CCD-18Co cells. Alternatively, a GLP-2 peptibody may, via its GLP-2 activity, decrease levels of caspase-3. Caspase 3 is a factor that is triggered by radiation. A GLP-2 peptibody may also inhibit Bcl-2 degradation, also triggered by radiation.

The GLP-2 peptibody may be administered before, or while, the patient is treated with radiation or radiotherapy. The GLP-2 peptibody may be administered after the patient is treated with radiation or radiotherapy. The GLP-2 peptibody, for example, comprising the amino acid sequence of SEQ ID NO: 1 or SEQ ID NO: 7, may be administered subcutaneously or intravenously. In various embodiments, multiple administrations are performed according to a dosing regimen. As used herein, the term “Q2D” means administration every two days, “Q3D” means administration every three days, etc. “QW” means administration every week. “BID” means administration twice a day. Dosing can be undertaken BID, once per day (QD), Q2D, Q3D, Q4D, Q5D, Q6D, QW, once every 8 days, once every 9 days, once every 10 days, once every 11 days, once every 12 days, once every 13 days, once every two weeks, once every 15 days, once every 16 days, or once every 17 days, once every three weeks, or once every month, for example. The GLP-2 peptibody (e.g., comprising the amino acid sequence of SEQ ID NO: 1 or SEQ ID NO: 7) may be administered subcutaneously according to a dosage regimen of between 0.02 to 3.0 mg/kg, 0.02 to 0.5 mg/kg, 0.04 to 0.45 mg/kg, 0.08 to 0.4 mg/kg, 0.10 to 0.35 mg/kg, 0.20 to 0.30 mg/kg, 0.02 to 0.05 mg/kg, 0.03 to 0.04 mg/kg, 0.05 to 0.10 mg/kg, 0.10 to 0.15 mg/kg, 0.2 to 0.3 mg/kg, 0.3 to 0.4 mg/kg, 0.4 to 0.5 mg/kg, 0.5 to 0.8 mg/kg, 0.7 to 1.0 mg/kg, 0.9 to 1.2 mg/kg, 1.0 to 1.5 mg/kg, 1.2 to 1.8 mg/kg, 1.5 to 2.0 mg/kg, 1.7 to 2.5 mg/kg, or 2.0 to 3.0 mg/kg once every 2-10 days, every 5-8 days, or every week (QW). The GLP-2 peptibody (e.g., comprising the amino acid sequence of SEQ ID NO: 7) may be administered subcutaneously according to a dosage regimen of between 0.2 to 1.4 mg/kg, 0.3 to 1.0 mg/kg, 0.4 to 0.9 mg/kg, 0.5 to 0.8 mg/kg, 0.3 to 0.7 mg/kg, 0.6 to 1.0 mg/kg, 0.2 to 0.4 mg/kg, 0.3 to 0.5 mg/kg, 0.4 to 0.6 mg/kg, 0.5 to 0.7 mg/kg, 0.6 to 0.8 mg/kg, 0.7 to 0.9 mg/kg, 0.8 to 1.0 mg/kg, 0.9 to 1.1 mg/kg, 1.0 to 1.2 mg/kg, 1.1 to 1.3 mg/kg, and 1.2 to 1.4 mg/kg, every week (QW) or every two weeks (Q2W).

Alternatively, the GLP-2 peptibody could be administered every three weeks or once a month, such as for maintenance purposes. The GLP-2 peptibody (e.g., comprising the amino acid sequence of SEQ ID NO: 7) may be administered subcutaneously according to a dosage regimen of between 0.2 to 1.4 mg/kg, 0.3 to 1.0 mg/kg, 0.4 to 0.9 mg/kg, 0.5 to 0.8 mg/kg, 0.3 to 0.7 mg/kg, 0.6 to 1.0 mg/kg, 0.2 to 0.4 mg/kg, 0.3 to 0.5 mg/kg, 0.4 to 0.6 mg/kg, 0.5 to 0.7 mg/kg, 0.6 to 0.8 mg/kg, 0.7 to 0.9 mg/kg, 0.8 to 1.0 mg/kg, 0.9 to 1.1 mg/kg, 1.0 to 1.2 mg/kg, 1.1 to 1.3 mg/kg, and 1.2 to 1.4 mg/kg, every three weeks or once a month.

The GLP-2 peptibody (e.g., comprising the amino acid sequence of SEQ ID NO: 1 or SEQ ID NO: 7) may be administered subcutaneously according to a dosage regimen of between 0.02 to 0.5 mg/kg, 0.04 to 0.45 mg/kg, 0.08 to 0.4 mg/kg, 0.10 to 0.35 mg/kg, 0.20 to 0.30 mg/kg every 5-8 days or every week (QW) for maintenance purposes. The GLP-2 peptibody comprising the amino acid sequence of SEQ ID NO: 1 or SEQ ID NO: 7 may be administered in a concentration of 10 to 100 mg/mL, 10 to 90 mg/mL, 20 to 80 mg/mL, 25 to 75 mg/mL, 30 to 70 mg/mL, 50 to 100 mg/mL, 60 to 90 mg/mL, about 75 mg/mL, 75 mg/mL, 10 to 20 mg/mL, 15 to 25 mg/mL, 12 to 18 mg/mL, 13-17 mg/mL, 14-16 mg/mL, about 15 mg/mL or 15 mg/mL.

The above dosing regimens may be conducted over six months to one year. GLP-2 peptibodies can be administered once a month after the initial dosage regimen for maintenance.

For example, the subcutaneous administration may be performed by injecting a composition into areas including, but not limited to, the thigh region, abdominal region, gluteal region, or scapular region. For such purposes, the formulation may be injected using a syringe. However, other devices for administration of the formulation are available such as injection devices (e.g., the Inject-ease™ and Genject™ devices); injector pens (such as the GenPen™); needleless devices (e.g., MediJector™ and BioJector™); and subcutaneous patch delivery systems. In some embodiments, a GLP-2 peptibody, (e.g., comprising the amino acid sequence of SEQ ID NO: 1 or SEQ ID NO: 7), or a pharmaceutical composition containing the same is administered intravenously.

In some embodiments, the method is effective to enhance intestinal absorption in the patient. In some embodiments, the method is effective to enhance intestinal absorption of nutrients, e.g., polypeptides, carbohydrates, fatty acids, vitamins, minerals, and water. In some embodiments, the method is effective to increase villus height in small intestine of the patient. In some embodiments, the method is effective to increase crypt depth in small intestine of the patient. In some embodiments, the method is effective to increase crypt organization in small intestine of the patient. In some embodiments, the method is effective to improve intestinal barrier function in the patient. These effects all may compensate for any radiation-induced cell damage that occurs in the small intestine and bowel.

In another aspect, the present invention provides a method for treating, ameliorating or preventing radiation-induced enteritis, and/or the effects thereof, to the gastrointestinal tract, comprising administering a GLP-2 peptibody, e.g., comprising the amino acid sequence of SEQ ID NO: 1 or SEQ ID NO: 7. A dosing regimen effective to treat or prevent radiation-induced enteritis in the patient may be used.

Radiation-induced enteritis may be reversed by GLP-2 peptibodies for similar reasons as discussed above with respect to radiation-induced damage to the gastrointestinal tract.

The GLP-2 peptibody, e.g., comprising the amino acid sequence of SEQ ID NO: 1 or SEQ ID NO: 7, may be administered subcutaneously or intravenously. The GLP-2 peptibody (e.g., comprising the amino acid sequence of SEQ ID NO: 1 or SEQ ID NO: 7) may be administered subcutaneously according to a dosage regimen of between 0.02 to 3.0 mg/kg, 0.02 to 0.5 mg/kg, 0.04 to 0.45 mg/kg, 0.08 to 0.4 mg/kg, 0.10 to 0.35 mg/kg, 0.20 to 0.30 mg/kg, 0.02 to 0.05 mg/kg, 0.03 to 0.04 mg/kg, 0.05 to 0.10 mg/kg, 0.10 to 0.15 mg/kg, 0.2 to 0.3 mg/kg, 0.3 to 0.4 mg/kg, 0.4 to 0.5 mg/kg, 0.5 to 0.8 mg/kg, 0.7 to 1.0 mg/kg, 0.9 to 1.2 mg/kg, 1.0 to 1.5 mg/kg, 1.2 to 1.8 mg/kg, 1.5 to 2.0 mg/kg, 1.7 to 2.5 mg/kg, or 2.0 to 3.0 mg/kg once every 2-14 days, every 5-8 days, or every week (QW). The GLP-2 peptibody (e.g., comprising the amino acid sequence of SEQ ID NO: 7) may be administered subcutaneously according to a dosage regimen of between 0.2 to 1.4 mg/kg, 0.3 to 1.0 mg/kg, 0.4 to 0.9 mg/kg, 0.5 to 0.8 mg/kg, 0.3 to 0.7 mg/kg, 0.6 to 1.0 mg/kg, 0.2 to 0.4 mg/kg, 0.3 to 0.5 mg/kg, 0.4 to 0.6 mg/kg, 0.5 to 0.7 mg/kg, 0.6 to 0.8 mg/kg, 0.7 to 0.9 mg/kg, 0.8 to 1.0 mg/kg, 0.9 to 1.1 mg/kg, 1.0 to 1.2 mg/kg, 1.1 to 1.3 mg/kg, and 1.2 to 1.4 mg/kg, every week (QW) or every two weeks (Q2W).

Alternatively, the GLP-2 peptibody could be administered every three weeks or once a month, such as for maintenance purposes. The GLP-2 peptibody (e.g., comprising the amino acid sequence of SEQ ID NO: 7) may be administered subcutaneously according to a dosage regimen of between 0.2 to 1.4 mg/kg, 0.3 to 1.0 mg/kg, 0.4 to 0.9 mg/kg, 0.5 to 0.8 mg/kg, 0.3 to 0.7 mg/kg, 0.6 to 1.0 mg/kg, 0.2 to 0.4 mg/kg, 0.3 to 0.5 mg/kg, 0.4 to 0.6 mg/kg, 0.5 to 0.7 mg/kg, 0.6 to 0.8 mg/kg, 0.7 to 0.9 mg/kg, 0.8 to 1.0 mg/kg, 0.9 to 1.1 mg/kg, 1.0 to 1.2 mg/kg, 1.1 to 1.3 mg/kg, and 1.2 to 1.4 mg/kg, every three weeks or once a month.

The GLP-2 peptibody (e.g., comprising the amino acid sequence of SEQ ID NO: 1 or SEQ ID NO: 7) may be administered subcutaneously according to a dosage regimen of between 0.02 to 0.5 mg/kg, 0.04 to 0.45 mg/kg, 0.08 to 0.4 mg/kg, 0.10 to 0.35 mg/kg, 0.20 to 0.30 mg/kg every 5-8 days or every week (QW) for maintenance purposes. The GLP-2 peptibody (e.g., comprising the amino acid sequence of SEQ ID NO: 1 or SEQ ID NO: 7) may be administered in a concentration of 10 to 100 mg/mL, 10 to 90 mg/mL, 20 to 80 mg/mL, 25 to 75 mg/mL, 30 to 70 mg/mL, 50 to 100 mg/mL, 60 to 90 mg/mL, about 75 mg/mL, 75 mg/mL, 10 to 20 mg/mL, 15 to 25 mg/mL, 12 to 18 mg/mL, 13-17 mg/mL, 14-16 mg/mL, about 15 mg/mL or 15 mg/mL.

For example, the subcutaneous administration may be performed by injecting a composition into areas including, but not limited to, the thigh region, abdominal region, gluteal region, or scapular region. For such purposes, the formulation may be injected using a syringe. However, other devices for administration of the formulation are available such as injection devices (e.g., the Inject-ease™ and Genject™ devices); injector pens (such as the GenPen™); needleless devices (e.g., MediJector™ and BioJector™); and subcutaneous patch delivery systems. In some embodiments, a GLP-2 peptibody, e.g., comprising the amino acid sequence of SEQ ID NO: 1 or SEQ ID NO: 7, or a pharmaceutical composition containing the same is administered intravenously.

In some embodiments, the method is effective to enhance intestinal absorption in the patient. In some embodiments, the method is effective to enhance intestinal absorption of nutrients, e.g., polypeptides, carbohydrates, fatty acids, vitamins, minerals, and water. In some embodiments, the method is effective to increase villus height in small intestine of the patient. In some embodiments, the method is effective to increase crypt depth in small intestine of the patient. In some embodiments, the method is effective to increase crypt organization in small intestine of the patient. In some embodiments, the method is effective to improve intestinal barrier function in the patient.

In another aspect is provided a method for treating a patient with short bowel syndrome presenting with colon in continuity with remnant small intestine comprising treating the patient with GLP-2 peptibody, e.g., comprising the amino acid sequence of SEQ ID NO: 1 or SEQ ID NO: 7, using a dosing regimen effective to treat the short bowel syndrome. In some embodiments, the GLP-2 peptibody is administered as a medicament for enhancing intestinal absorption in short bowel syndrome patients presenting with at least about 25% colon-in-continuity with remnant small intestine. In some embodiments, the remnant small intestine has a length of at least 25 cm, at least 50 cm, at least 75 cm, at least 100 cm, or at least 125 cm. In some embodiments, the method is effective to enhance intestinal absorption in the patient. In some embodiments, the method is effective to enhance intestinal absorption of nutrients, e.g., polypeptides, carbohydrates, fatty acids, vitamins, minerals, and water. In some embodiments, the method is effective to increase villus height in the small intestine of the patient. In some embodiments, the method is effective to increase crypt depth in the small intestine of the patient. In some embodiments, the patient is dependent on parenteral nutrition. The method may be effective to decrease fecal wet weight, increase urine wet weight, increase energy absorption across the small intestine (e.g., absorption of one of more of polypeptides, carbohydrates, fatty acids), increase water absorption across the small intestine, reduce parenteral nutrition support, or eliminate the need for parenteral nutrition.

A dosing regimen may be used that is effective to treat short bowel syndrome with colon-in-continuity. The GLP-2 peptibody, comprising the amino acid sequence of SEQ ID NO: 1 or SEQ ID NO: 7, may be administered subcutaneously or intravenously. In various embodiments, multiple administrations are performed according to a dosing regimen. As used herein, the term “Q2D” means administration every two days, “Q3D” means administration every three days, etc. “QW” means administration every week. “BID” means administration twice a day. Dosing can be undertaken BID, once per day (QD), Q2D, Q3D, Q4D, Q5D, Q6D, QW, once every 8 days, once every 9 days, once every 10 days, once every 11 days, once every 12 days, once every 13 days, once every two weeks, once every 15 days, once every 16 days, or once every 17 days, once every three weeks, or once every month, for example. The GLP-2 peptibody (comprising the amino acid sequence of SEQ ID NO: 1 or SEQ ID NO: 7, for example) may be administered subcutaneously according to a dosage regimen of between 0.02 to 3.0 mg/kg, 0.02 to 0.5 mg/kg, 0.04 to 0.45 mg/kg, 0.08 to 0.4 mg/kg, 0.10 to 0.35 mg/kg, 0.20 to 0.30 mg/kg, 0.02 to 0.05 mg/kg, 0.03 to 0.04 mg/kg, 0.05 to 0.10 mg/kg, 0.10 to 0.15 mg/kg, 0.2 to 0.3 mg/kg, 0.3 to 0.4 mg/kg, 0.4 to 0.5 mg/kg, 0.5 to 0.8 mg/kg, 0.7 to 1.0 mg/kg, 0.9 to 1.2 mg/kg, 1.0 to 1.5 mg/kg, 1.2 to 1.8 mg/kg, 1.5 to 2.0 mg/kg, 1.7 to 2.5 mg/kg, or 2.0 to 3.0 mg/kg once every 2-14 days, every 5-8 days, or every week (QW). The GLP-2 peptibody (e.g., comprising the amino acid sequence of SEQ ID NO: 7) may be administered subcutaneously according to a dosage regimen of between 0.2 to 1.4 mg/kg, 0.3 to 1.0 mg/kg, 0.4 to 0.9 mg/kg, 0.5 to 0.8 mg/kg, 0.3 to 0.7 mg/kg, 0.6 to 1.0 mg/kg, 0.2 to 0.4 mg/kg, 0.3 to 0.5 mg/kg, 0.4 to 0.6 mg/kg, 0.5 to 0.7 mg/kg, 0.6 to 0.8 mg/kg, 0.7 to 0.9 mg/kg, 0.8 to 1.0 mg/kg, 0.9 to 1.1 mg/kg, 1.0 to 1.2 mg/kg, 1.1 to 1.3 mg/kg, and 1.2 to 1.4 mg/kg, every week (QW) or every two weeks (Q2W).

Alternatively, the GLP-2 peptibody could be administered every three weeks or once a month, such as for maintenance purposes. The GLP-2 peptibody (e.g., comprising the amino acid sequence of SEQ ID NO: 7) may be administered subcutaneously according to a dosage regimen of between 0.2 to 1.4 mg/kg, 0.3 to 1.0 mg/kg, 0.4 to 0.9 mg/kg, 0.5 to 0.8 mg/kg, 0.3 to 0.7 mg/kg, 0.6 to 1.0 mg/kg, 0.2 to 0.4 mg/kg, 0.3 to 0.5 mg/kg, 0.4 to 0.6 mg/kg, 0.5 to 0.7 mg/kg, 0.6 to 0.8 mg/kg, 0.7 to 0.9 mg/kg, 0.8 to 1.0 mg/kg, 0.9 to 1.1 mg/kg, 1.0 to 1.2 mg/kg, 1.1 to 1.3 mg/kg, and 1.2 to 1.4 mg/kg, every three weeks or once a month.

The GLP-2 peptibody (e.g., comprising the amino acid sequence of SEQ ID NO: 1 or SEQ ID NO: 7) may be administered subcutaneously according to a dosage regimen of between 0.02 to 0.5 mg/kg, 0.04 to 0.45 mg/kg, 0.08 to 0.4 mg/kg, 0.10 to 0.35 mg/kg, 0.20 to 0.30 mg/kg every 5-8 days or every week (QW) for maintenance purposes. The GLP-2 peptibody (e.g., comprising the amino acid sequence of SEQ ID NO: 1 or SEQ ID NO: 7) may be administered in a concentration of 10 to 100 mg/mL, 10 to 90 mg/mL, 20 to 80 mg/mL, 25 to 75 mg/mL, 30 to 70 mg/mL, 50 to 100 mg/mL, 60 to 90 mg/mL, about 75 mg/mL, 75 mg/mL, 10 to 20 mg/mL, 15 to 25 mg/mL, 12 to 18 mg/mL, 13-17 mg/mL, 14-16 mg/mL, about 15 mg/mL or 15 mg/mL.

In some embodiments, a GLP-2 peptibody, e.g., comprising the amino acid sequence of SEQ ID NO: 1 or SEQ ID NO: 7, or a pharmaceutical composition containing the same is administered subcutaneously. For example, the subcutaneous administration may be performed by injecting a composition into areas including, but not limited to, the thigh region, abdominal region, gluteal region, or scapular region. In some embodiments, a GLP-2 peptibody, (comprising the amino acid sequence of SEQ ID NO: 1 or SEQ ID NO: 7, for example), or a pharmaceutical composition containing the same is administered intravenously.

Similar to above, GLP-2 peptibodies may be used to treat an individual suffering from gastro-intestinal disorders, including the upper gastrointestinal tract of the esophagus by administering an effective amount of a GLP-2 analogue, or a salt thereof as described herein. The stomach and intestinal-related disorders include ulcers of any etiology (e.g., peptic ulcers, drug-induced ulcers, ulcers related to infections or other pathogens), digestion disorders, malabsorption syndromes, short-bowel syndrome, cul-de-sac syndrome, inflammatory bowel disease, celiac sprue (for example, arising from gluten induced enteropathy or celiac disease), tropical sprue, hypogammaglobulinemic sprue, enteritis, ulcerative colitis, small intestine damage and chemotherapy induced diarrhea/mucositis (CID). Individuals who would benefit from increased small intestinal mass and consequent and/or maintenance of normal small intestine mucosal structure and function are candidates for treatment with GLP-2 peptibodies. Particular conditions that may be treated with GLP-2 peptibodies include the various forms of sprue including celiac sprue, which results from a toxic reaction to alpha-gliadin from heat, may be a result of gluten-induced enteropathy or celiac disease, and is marked by a significant loss of villae of the small bowel; tropical sprue, which results from infection and is marked by partial flattening of the villae; hypogammaglobulinemic sprue, which is observed commonly in patients with common variable immunodeficiency or hypogammaglobulinemia and is marked by significant decrease in villus height. The therapeutic efficacy of the GLP-2 peptibody treatment may be monitored by enteric biopsy to examine the villus morphology, by biochemical assessment of nutrient absorption, by patient weight gain, or by amelioration of the symptoms associated with these conditions.

GLP-2 peptibodies may also be administered to prevent or treat damage to the gastrointestinal tract during chemotherapy. Chemotherapy-induced damage to the small intestinal mucosa is clinically often referred to as gastrointestinal mucositis and is characterized by absorptive and barrier impairments of the small intestine. Gastrointestinal mucositis after cancer chemotherapy is an increasing problem that is essentially untreatable once established, although it gradually remits. Studies conducted with the commonly used cytostatic cancer drugs 5-FU and irinotecan have demonstrated that effective chemotherapy with these drugs predominantly affects structural integrity and function of the small intestine. Administration of GLP-2 peptibodies may reverse damage to the small intestine and preserve its structural integrity and function.

In various embodiments of the above treatment methods, particular doses or amounts to be administered may vary, for example, depending on the nature and/or extent of the desired outcome, on particulars of route and/or timing of administration, and/or on one or more characteristics (e.g., weight, age, personal history, genetic characteristic, lifestyle parameter, severity of cardiac defect and/or level of risk of cardiac defect, etc., or combinations thereof). Such doses or amounts can be determined by those of ordinary skill. In some embodiments, an appropriate dose or amount is determined in accordance with standard clinical techniques. Alternatively or additionally, in some embodiments, an appropriate dose or amount is determined through use of one or more in vitro or in vivo assays to help identify desirable or optimal dosage ranges or amounts to be administered.

In various embodiments of the above treatment methods, GLP-2 peptibody is administered at a therapeutically effective amount. Generally, a therapeutically effective amount is sufficient to achieve a meaningful benefit to the subject (e.g., prophylaxis, treating, modulating, curing, preventing and/or ameliorating the underlying disease or condition). Generally, the amount of a therapeutic agent (e.g., a GLP-2 peptibody) administered to a subject in need thereof will depend upon the characteristics of the subject. Such characteristics include the condition, disease severity, general health, age, sex and body weight of the subject. One of ordinary skill in the art will be readily able to determine appropriate dosages depending on these and other related factors. In addition, both objective and subjective assays may optionally be employed to identify optimal dosage ranges. In some particular embodiments, appropriate doses or amounts to be administered may be extrapolated from dose-response curves derived from in vitro or animal model test systems.

In various embodiments of the above treatment methods, a therapeutically effective amount is commonly administered in a dosing regimen that may comprise multiple unit doses. For any particular therapeutic protein, a therapeutically effective amount (and/or an appropriate unit dose within an effective dosing regimen) may vary, for example, depending on route of administration, on combination with other pharmaceutical agents. Also, the specific therapeutically effective amount (and/or unit dose) for any particular patient may depend upon a variety of factors including the disorder being treated and the severity of the disorder; the activity of the specific pharmaceutical agent employed; the specific composition employed; the age, body weight, general health, sex and diet of the patient; the time of administration, route of administration, and/or rate of excretion or metabolism of the specific fusion protein employed; the duration of the treatment; and like factors as is well known in the medical arts.

In various embodiments of the above treatment methods, a GLP-2 peptibody is administered in combination with one or more known therapeutic agents. In some embodiments, the known therapeutic agent(s) is/are administered according to its standard or approved dosing regimen and/or schedule. In some embodiments, the known therapeutic agent(s) is/are administered according to a regimen that is altered as compared with its standard or approved dosing regimen and/or schedule. In some embodiments, such an altered regimen differs from the standard or approved dosing regimen in that one or more unit doses is altered (e.g., reduced or increased) in amount, and/or in that dosing is altered in frequency (e.g., in that one or more intervals between unit doses is expanded, resulting in lower frequency, or is reduced, resulting in higher frequency).

For ECF, exemplary therapeutic agents that may be administered in combination with GLP-2 peptibodies include corticosteroids, antibiotics and acid reducers. For obstructive jaundice, exemplary therapeutic agents that may be administered in combination with GLP-2 peptibodies include corticosteroids and antibiotics.

In various embodiments of the above treatment methods, multiple different GLP-2 peptibodies may be administered together. Further, GLP-2 peptibodies may be concurrently administered with Gattex, teduglutide or GLP-2 peptide.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1: Molecular Weight and FcRn Binding of GLP-2 Peptibodies

Binding to the Fc neonatal receptor (FcRN) allows for recycling of the molecules and leads to an extended in vivo serum half-life of the Fc fusion proteins. Recycling occurs as the molecules are passively taken into the cells and the pH of the endosomes is lower. That leads to binding of the Fc portion of the molecule to the FcRN. When the FcRN recycles back to the surface of the cell, the pH is then neutral and the protein is released back into the serum.

Binding to the extracellular domain of the FcRN was measured by surface plasmon resonance (SPR) using a Biacore system. Direct immobilization with FcRn was achieved via amine coupling of a CM5 chip with FcRn under the following conditions:
i) hFcRn (expressed and purified in house) is diluted in Acetate buffer pH 5.0 to 5 μg/mL.
ii) Immobilize 5 μg/mL of FcRn with target of 500 RU on CM5 chip in PBS pH 7.0
iii) Final response 454 RU
iv) Running buffer: PBS-P+, pHed to 5.5

The kinetic binding study was done using the following protocol. Samples were diluted in PBS-P+ to 50, 25, 12.5, 6.25, 3.125, 1.56, 0.78, 0.39, 0 nM. The parameters were set as follows:
i) Association and Dissociation 300 s at Flow rate 30 μL/min
ii) Regeneration with 25 mM Tris, 150 mM NaCl pH 8.0 40s at 60 μL/min

A measurement of the binding of the GLP-2 peptibodies to the Fc neonatal receptor (FcRN) was undertaken at pH 5.5 and pH 7.4. GLP-2 peptibody 0, with albumin instead of Fc, has a substantially higher K_{D}. The results are shown in Table 1 below.

TABLE 1

| GLP-Peptibody | MW | FcRN K_{D} at pH 5.5 | FcRN K_{D} at pH 7.4 |
|---|---|---|---|
| A | 58.4 | 1.38 | No binding in range tested. |
| B | 48.97 | 1.70 | No binding in range tested. |
| E | 60.66 | 2.04 | No binding in range tested. |
| J | 65.75 | 2.90 | No binding in range tested. |
| K | 60.29 | 1.95 | No binding in range tested. |
| L | 59.19 | 1.72 | No binding in range tested. |
| M | 59.65 | 1.81 | No binding in range tested. |
| O | 71.36 | 1373 | No binding in range tested. |

### Example 2: Protein Stability Analysis

Each of the GLP-2 peptibodies was tested by determining melting temperature with nanodifferential scanning fluorimetry (NanoDSF). NanoDSF is a measurement of protein stability over a range of temperatures, with a temperature ramp employed. The stability of tryptophan is measured by fluorescence, as reflected in a ratio of fluorescence at 350 nm to fluorescence at 330 nm. From the assay, one or more melting temperatures are determined. Because a protein in a certain state is understood to have a melting temperature, the number of melting temperatures observed reflects the number of different states. GLP-2 peptibodies A, B, E, J, K, L, and M have two states, as shown in Table 2 below.

A SEC-MALS assay was performed to determine the primary state (main peak) and its molecular weight. As shown in Table 2 below, the GLP-2 peptibodies A, B, E, J, K, L, M, and O (Fc fusions) eluted at a molecular weight indicative of a dimer. The GLP-2 peptibody O (albumin fusion) eluted at a molecular weight indicative of a monomer.

TABLE 2

| | | SEC-MALS |
|---|---|---|
| GLP-Peptibody | NanoDSF | Zenix C-150 |
| A | 1 = 67.0° C. | Not tested |
| | 2 = 80° C. | |
| B | 1 = 67.1° C. | 85% main peak, 158,800 g/mol |
| | 2 = 79.9° C. | |
| E | 1 = 67.5° C. | Not tested |
| | 2 = 80° C. | |
| J | 1 = 68.1° C. | 98% main peak, 168,400 g/mol |
| | 2 = 82.0° C. | |
| K | 1 = 67.5° C. | 80.2% main peak, 149,900 g/mol |
| | 2 = 79.7° C. | |
| L | 1 = 67.5° C. | 87.4% main peak, 148,000 g/mol |
| | 2 = 80° C. | |
| M | 1 = 67.3° C. | 81.4% main peak, 148,500 g/mol |
| | 2 = 79.9° C. | |
| O | 1 = 57.6° C. | 89.5% main peak, 76,500 g/mol |

### Example 3: In Vitro Potency of GLP-2 Peptibodies

The EC50 of GLP-2 peptibodies was assayed in vitro using the cAMP Hunter™ eXpress GLP2R CHO-K1 GPCR assay from DiscoverX. The cAMP Hunter™ assay is based on enzyme fragment complementation (EFC). In EFC assay, the enzyme donor is fused to cAMP. Increased intracellular cAMP due to GLP2R activation competes with ED-cAMP for antibody. Unbound ED-cAMP complements the enzyme acceptor to form active beta galactosidase, which subsequently produces a luminescent signal.

The CHO-K1 cell line used is overexpressing human GLP-2R (Genbank accession number NM004246.1). The peptide GLP-2[A2G] was used as a control. Cells were treated with various dilutions of GLP-2[A2G] peptide and GLP-2 peptibodies listed in Table 3. Their activities were assayed via measurement of the concentration of cAMP in the media. Sigmoidal curve fitting was undertaken to arrive at EC50 values, as shown in Table 3 below.

TABLE 3

Peptide

| GLP-Peptibody | EC50 (nM) | R² |
|---|---|---|
| GLP-2[A2G] | 0.59 | 0.99 |
| A | 128.3 | 0.99 |
| B | 8.27 | 0.99 |
| E | 2.43 | 0.99 |
| J | 3.23 | 0.99 |
| K | 2.87 | 0.99 |
| L | 6.16 | 0.98 |
| M | 4.51 | 0.97 |
| O albumin fusion | 10.55 | 0.98 |
| P albumin fusion | 150.9 | 0.99 |

The EC50 values for the GLP-2 peptibodies were substantially greater than that of GLP-2[A2G]. However, in vitro potency is only reduced slightly for some GLP-2 peptibodies, such as GLP-2 peptibody K where the reduction of activity is only about five-fold. GLP-2 peptibody K has 20% of the in vitro activity of GLP-2[A2G]. GLP-2 peptibody E has 24% of the in vitro activity of GLP-2[A2G]. GLP-2 peptibody E has 18% of the in vitro activity of GLP-2[A2G]. GLP-2 peptibody B has 7% of the in vitro activity of GLP-2[A2G].

Pharmacokinetic studies were then performed, as discussed below, to assay for how long the GLP-2 peptibodies are active in vivo.

### Example 4: Rat Pharmacokinetic Studies—Intravenous Dosing

In the rat, four pharmacokinetic parameters were measured for Gattex® (a GLP-2 peptide having the A2G mutation): CL, Vc, Vt and Q. The same pharmacokinetic parameters were also measured for GLP-2 peptibodies A, B, E, J, K, L, M, O and P. The data is shown in Table 4. Male Sprague-Dawley rats (3 animals per group) were injected intravenously either via a jugular vein or tail vein catheter. A singles dose of test article was injected at a dose level of 1 mg/ml. The test articles were formulated in PBS pH 7.4 at a concentration of Blood samples were taken 0.083, 0.167, 0.33, 0.5, 1, 2, 6, 24, 48, 72, 120, 168, 240, and 336 hours post dose. Blood samples were collected into heparinized tubes and centrifuged for 5 minutes at 2000×g within 10 minutes of collection. 100 μL of plasma were transferred to a 1.5 ml Eppendorf tube containing 2 μL of 50 mM PMSF. After mixing, the plasma samples were frozen at −80° C. until analysis.

TABLE 4

| | Peptide | | | | | | | |
|---|---|---|---|---|---|---|---|---|
| GLP-Peptibody | CL (mL/day/kg) | | Vc (mL/kg) | | Vt (mL/kg) | | Q (mL/day/kg) | |
| Gattex | 33,391 | (10%) | 2,235 | (10%) | NA | (<0.1) | NA | (<0.1) |
| A | 57 | (7.1%) | 43 | (17.8%) | 79 | (16%) | 58 | (15%) |
| B | 48 | (11%) | 31 | (17%) | 76 | (18%) | 58 | (18%) |
| E | 72 | (31%) | 21 | (15%) | 41 | (15%) | 69 | (25%) |
| J | 57.8 | (6%) | 37.6 | (12%) | 22 | (14%) | 15.6 | (15%) |
| K | 53.7 | (4%) | 42.2 | (4%) | 46.4 | (13%) | 61 | (22%) |
| L | 67.3 | (9%) | 37.8 | (7%) | 538 | (6.1%) | 19 | (10%) |
| M | 38.3 | (71%) | 12 | (9%) | 29.4 | (7%) | 183 | (8.3%) |
| O | 130 | (18%) | 43.2 | (9%) | 54 | (14%) | 1380 | (22%) |
| P | 170 | (23%) | 38.4 | (11%) | 43.9 | (21%) | 707 | (13%) |

### Example 5: Rat Pharmacokinetic Studies—Subcutaneous Dosing

In the rat, four pharmacokinetic parameters were measured for Gattex® (a GLP-2 peptide having the A2G mutation): CL, Vc, Vt and Q. The same pharmacokinetic parameters were also measured for GLP-2 peptibodies A, B, E, J, K, L, M, O and P. The data is shown in Table 5. Male Sprague-Dawley rats (3 animals per group) were injected subcutaneously into the intra-scapular region of the animal. A singles dose of test article was injected at a dose level of 1 mg/ml. The test articles were formulated in PBS pH 7.4 at a concentration of Blood samples were taken 0.083, 0.167, 0.33, 0.5, 1, 2, 6, 24, 48, 72, 120, 168, 240, and 336 hours post dose. Blood samples were collected into heparinized tubes and centrifuged for 5 minutes at 2000×g within 10 minutes of collection. 100 μL of plasma were transferred to a 1.5 ml Eppendorf tube containing 2 μL of 50 mM PMSF. After mixing the plasma samples were frozen at −80T until analysis. Meso Scale Discovery (MSD) ELISA was undertaken to assay for the concentration of the GLP-2 peptibodies.

A sandwich immunoassay was developed using either an anti-Human IgG1 Fc antibody or an anti-human albumin antibody for capture of the peptibody and a sulfotag labeled anti GLP-2 antibody for detection.

TABLE 5

| | Peptide | | | | | | | |
|---|---|---|---|---|---|---|---|---|
| GLP-Peptibody | CL (mL/day/kg) | | Vc (mL/kg) | | Vt (mL/kg) | | Q (mL/day/kg) | |
| Gattex ® | 51,649 | (10%) | 1,794 | (10%) | NA | (<0.1) | NA | (<0.1) |
| A (0.45 ka/day, | 70.7 | (11%) | 109 | (18%) | 81 | (8%) | 78 | (18%) |
| 14%) | | | | | | | | |
| B (0.45 ka/day, | 43 | (19%) | 73 | (16%) | 68 | (13%) | 52 | (18%) |
| 16%) | | | | | | | | |
| E (0.63 ka/day, | 121 | (10%) | 205 | (19%) | NA | (<0.1) | NA | (<0.1) |
| 11%) | | | | | | | | |
| J (0.93 ka/day, | 195 | (22%) | 193 | (9.8%) | 99.5 | (<0.1) | 0.3 | (<0.1) |
| 9.6%) | | | | | | | | |
| K (0.62 ka/day, | 68 | (13%) | 114 | (19%) | NA | (<0.1) | NA | (<0.1) |
| 19%) | | | | | | | | |
| L (0.63 ka/day, | 80.6 | (21%) | 127 | (22%) | NA | (<0.1) | NA | (<0.1) |
| 19%) | | | | | | | | |
| M (0.78 ka/day, | 60.2 | (10%) | 91.5 | (17%) | NA | (<0.1) | NA | (<0.1) |
| 24%) | | | | | | | | |
| O (1.26 ka/day, | 742 | (31%) | 565 | (27%) | NA | (<0.1) | NA | (<0.1) |
| 28%) | | | | | | | | |

| | | | | |
|---|---|---|---|---|
| P | NA | NA | NA | NA |

### Example 5: Expression and Purification of GLP-2 Peptibody B264

GLP-2 peptibody B264 coding sequence was cloned into a plasmid for expression in a CHO host cell line. GLP-2 peptibody B264 was purified using a MAb Select Sure® column having a 21 cm bed and 400 mL resin. DPBS was used as both an equilibration buffer and a wash buffer. For elution, 100 mM glycine at pH 3.0 was used. The neutralization buffer was 1 M Tris-HCl at pH 9.0, with 1.45 mL used per 45 mL elution.

An Akta protein purification system was then used for purification. 5 column volumes of DPBS was used for equilibration. 6 L of sample was loaded at a rate of 35 mL per minute. The column was washed with 10 column volumes of DPBS. Elution was undertaken using 5-10 column volumes of 100 mM glycine pH 3.0, in 45 mL fractions neutralized with 1.45 mL of 1 M Tris-HCl at pH 9.0. The elution fractions were combined and dialyzed against PBS pH 7.4 Fisher (diluted from 10×PBS), at 70 mL sample per 2.5 L dPBS while stirring overnight at 4° C.

Total protein was assayed by each of Nanodrop, Bradford and BCA. The final concentration of GLP peptibody B264 was 11 mg/mL in a total volume of 170 mL. The total yield was 1.87 grams. The endotoxin level was 1.72 EU/mL or about 0.15 EU/mg.

Stability analysis was then performed using SEC-MALS and NanoDSF. For SEC-MALS, a Sepax Zenix C-150 column was used. The mobile phase buffer was 1×PBS with a final concentration of 400 mM NaCl. The flow rate was 0.8 mL per minute. 20 micrograms of total protein was injected. For NanoDSF, 10 microliters of sample was used, without normalization of the samples. The data is shown below in Table 6.

TABLE 6

| Sample of GLP-2 | | Concentration | | | Thermal Stability |
|---|---|---|---|---|---|
| Peptibody B264 | | at thaw | | SEC | (NanoDSF) |
| 11 | mg/mL | 10.91 | mg/mL | 2.1% HMW | 1 = 67.5° C. |
| | | | | 80.5% Main Peak | 2 = 74.7° C. |
| | | | | 17.4% LMW | |
| 5 | mg/mL | 5.15 | mg/mL | 2% HMW | 1 = 67.2° C. |
| | | | | 77.9% Main Peak | 2 = 75.0° C. |
| | | | | 20.1% LMW | |
| 1.5 | mg/mL | 1.36 | mg/mL | 2.1% HMW | 1 = 67.0° C. |
| | | | | 77.2% Main Peak | 2 = 74.8° C. |
| | | | | 20.8% LMW | |
| 0.5 | mg/mL | 0.31 | mg/mL | 82.8% Main Peak | 1 = 67.1° C. |
| | | | | 17.2% LMW | 2 = 75.2° C. |

### Example 6: Expression and Purification of GLP-2 Peptibody K274

GLP-2 peptibody K274 coding sequence was cloned into a plasmid for expression in a CHO host cell line. GLP-2 peptibody K274 was purified using a MAb Select Sure® column having a 17 cm bed and 300 mL resin. DPBS was used as both an equilibration buffer and a wash buffer. For elution, 100 mM glycine at pH 3.0 was used. The neutralization buffer was 1 M Tris-HCl at pH 9.0, with 1.45 mL used per 45 mL elution.

An Akta protein purification system was then used for purification. 5 column volumes of DPBS was used for equilibration. 6 L of sample was loaded at a rate of 35 mL per minute. The column was washed with 10 column volumes of DPBS. Elution was undertaken using 5-10 column volumes of 100 mM glycine pH 3.0, in 45 mL fractions neutralized with 1.45 mL of 1 M Tris-HCl at pH 9.0.

The elution fractions were combined and dialyzed against PBS pH 7.4 Fisher (diluted from 10×PBS), at 70 mL sample per 2.5 L dPBS while stirring overnight at 4° C.

Total protein was assayed by each of Nanodrop, Bradford and BCA. The final concentration of GLP peptibody B264 was 11 mg/mL in a total volume of 170 mL. The total yield was 1.87 grams.

Stability analysis was then performed using SEC-MALS and NanoDSF. For SEC-MALS, a Sepax Zenix C-150 column was used. The mobile phase buffer was 1×PBS with a final concentration of 400 mM NaCl. The flow rate was 0.8 mL per minute. 20 micrograms of total protein was injected. For NanoDSF, 10 microliters of sample was used, without normalization of the samples. The results are shown in Table 7 below.

TABLE 7

| | | |
|---|---|---|
| Sample of GLP-2 | | Thermal Stability |
| Peptibody B264 | SEC | (NanoDSF) |

| | | | |
|---|---|---|---|
| 7.5 | mg/mL | 80% Main Peak | 1 = 67.8° C. |
| | | 19.9% LMW | 2 = 80.4° C. |
| 5 | mg/mL | 79.2% Main Peak | 1 = 67.7° C. |
| | | 20.8% LMW | 2 = 80.7° C. |
| 1.5 | mg/mL | 78.6% Main Peak | 1 = 67.6° C. |
| | | 21.4% LMW | 2 = 80.2° C. |
| 0.5 | mg/mL | 2.9% HMW | 1 = 67.6° C. |
| | | 77.4% Main Peak | 2 = 80.2° C. |
| | | 19.7% LMW | |

### Example 7: Dimer/Monomer Analysis of GLP-2 Peptibody B264 and GLP-2 Peptibody K274

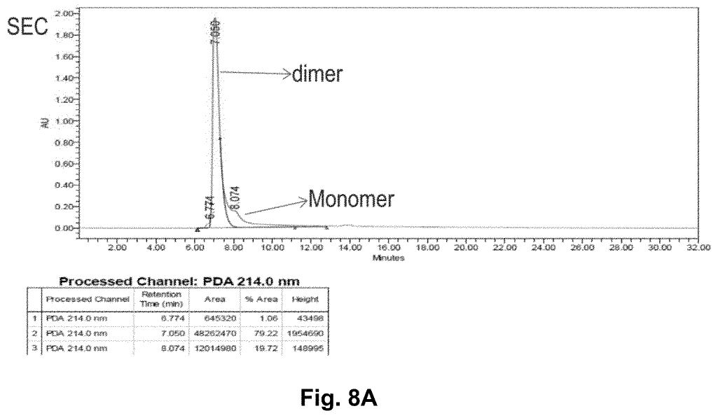

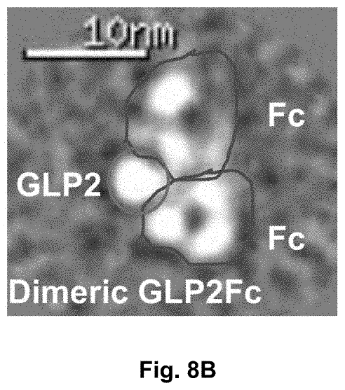

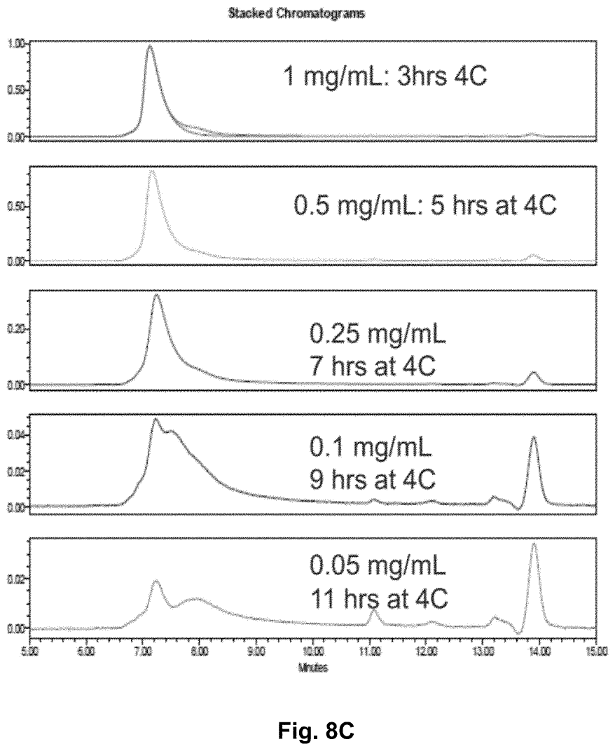

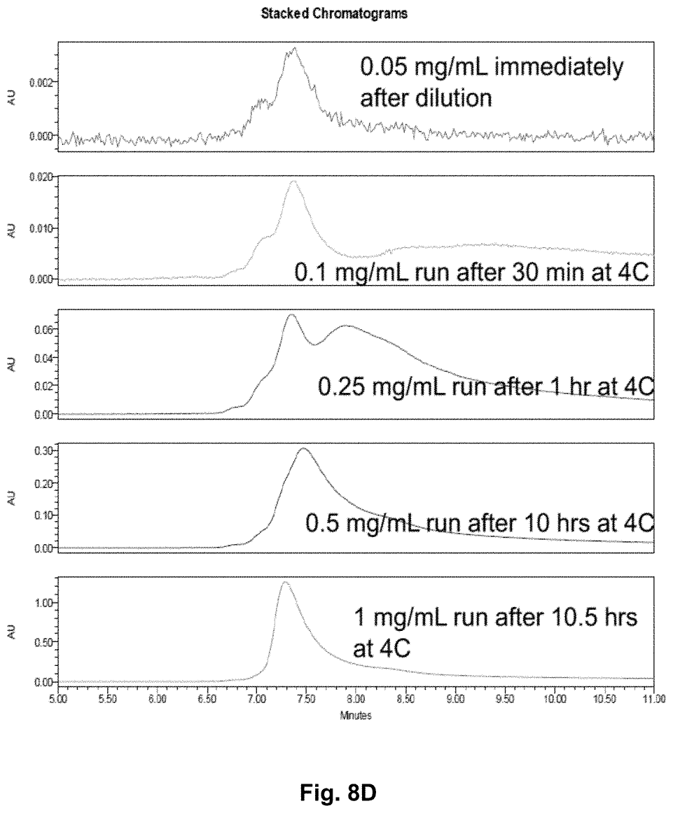

A SEC-MALS analysis of GLP-2 peptibody B264 and GLP-2 peptibody K274 showed a molecular weight of about 140,000 g/mol, which corresponds to the size of a dimer. AUC and EM analyses confirmed that a dimer was present. The expected molecular weight of a monomer of GLP-2 peptibody B264 is 58,970 and the expected molecular weight of GLP-2 peptibody K274 is 60,290. The results of the SEC-MALS analysis is shown in FIG. 8A, with a peak corresponding to the dimer appearing at about 7 minutes and a peak corresponding to the monomer appearing at about 8 minutes. A dilution effect of the SEC was observed to be in the monomer/dimer transition range.

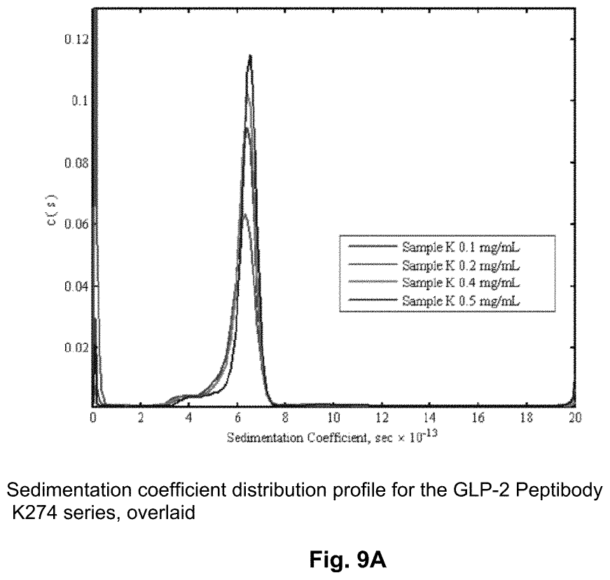

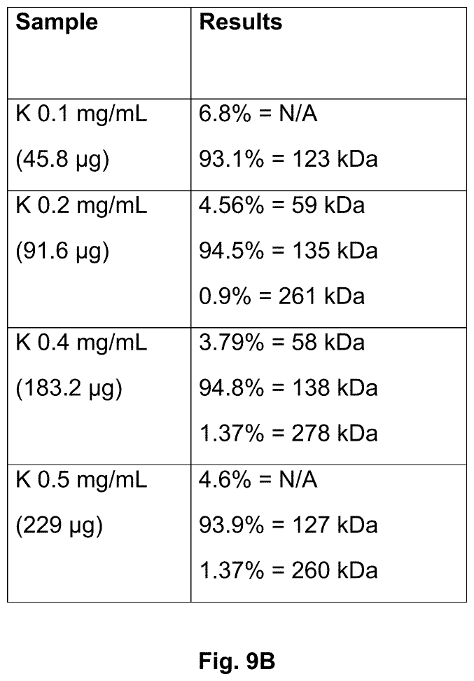

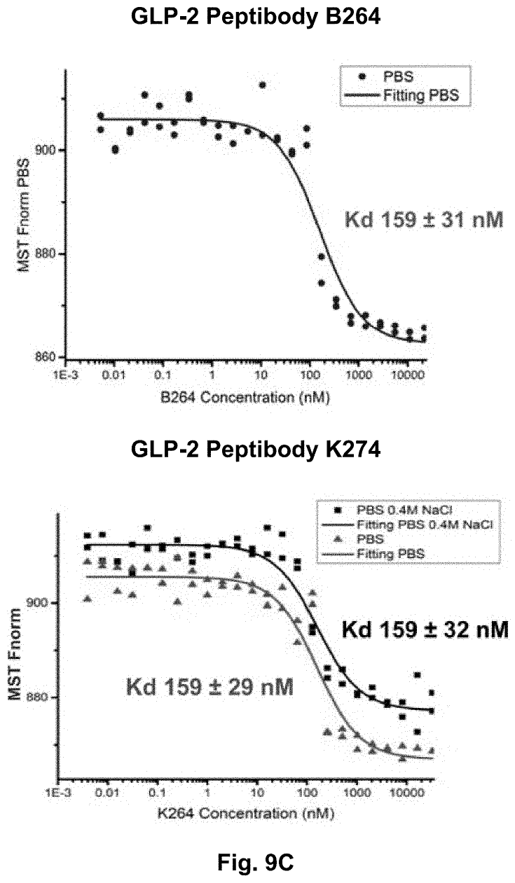

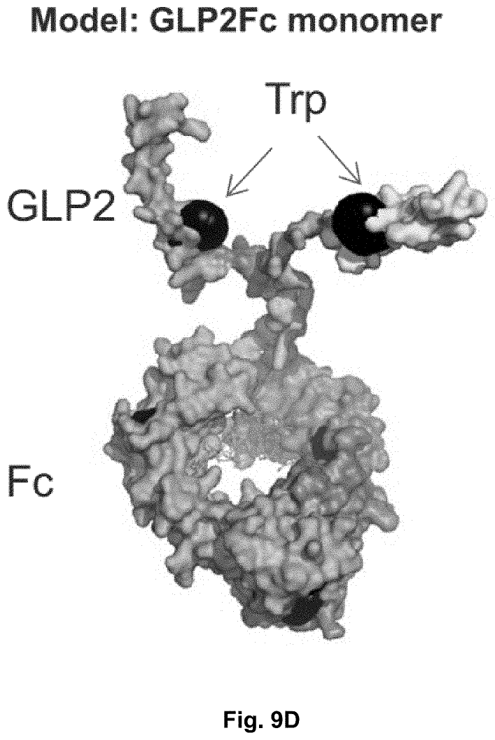

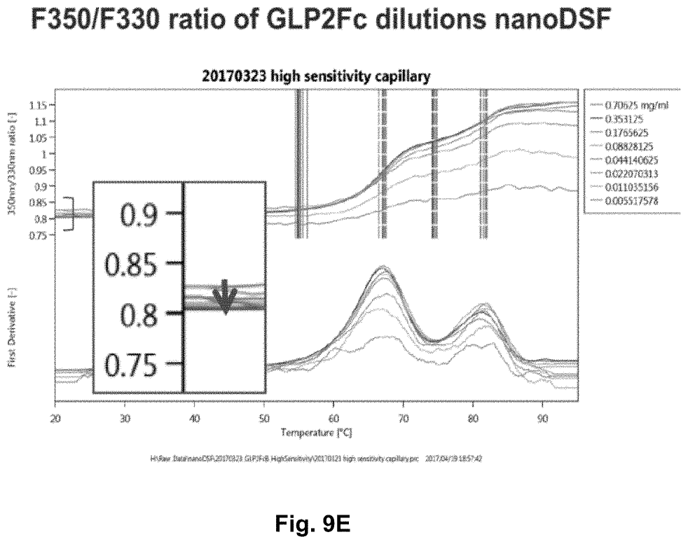

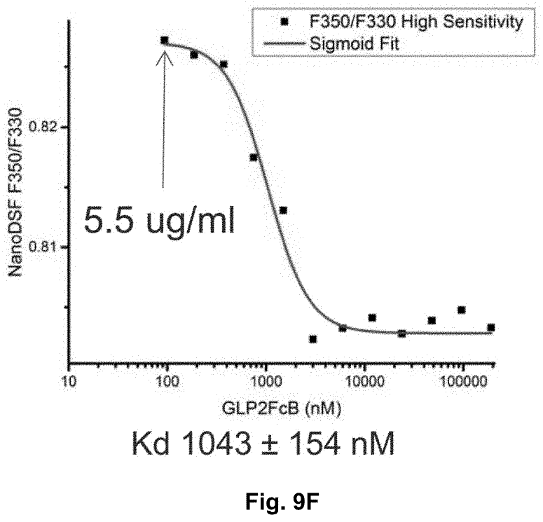

The results of the EM analysis of dimer GLP-2-Fc (GLP-2 peptibody B) is shown in FIG. 8B. More dimer appears at decreasing concentrations and increasing time at 4° C., as shown in FIGS. 8C and 8D with respect to GLP-2 peptibody K. The results of AUC and SEC analyses are shown in FIGS. 9A and 9B for GLP-2 peptibody K. FIG. 9A shows an overlay of the sedimentation coefficient (SEC) distribution profile. The samples are in the 1-8 μM range, however during the SEC analysis, the samples are diluted on the column such that they fall into the monomer-dimer transition range. In addition, 4 μL of 11.3 mg/mL of sample was injected for SEC analysis and each drop fractionated, with A280 measured on Nanodrop to show that the sample concentration on SEC falls into the monomer-dimer transition range. To summarize the above, GLP-2-Fc was observed as a dimer in the AUC and SEC-MALS assays. The monomer/dimer ratio changed based on concentration, according to SEC-MALS.

Microscale thermophoresis (MST) and nano differential scanning fluorimetry (NanoDSF) were performed to characterize the dimer-monomer transition. MST was used to determine the monomer/dimer equilibrium dissociation constant Kd. MST is based on thermodriven diffusion of molecules while NanoDSF is based on Trp fluorescence and is commonly used for thermostability Tm. MST was performed on both GLP-2 peptibody B264 and GLP-2 peptibody K274, as shown in FIG. 9C. The Kd for GLP-2 peptibody B264 was 159±31 nM. The Kd for GLP-2 peptibody K274 was 159±29 nM in PBS and 159±32 nM in PBS with 0.4 M NaCl. Also, the Kd for teduglutide is 24±3 μM with MST.

In the NanoDSF assay, room temperature is used and one tryptophan in GLP-2 is targeted that potentially undergoes conformational changes during GLP-2-Fc self-association. See FIG. 9D. Only the tryptophan fluorescence from the protein contributes to the signal. If tryptophan is buried or stable, the peak is at 330 nm and if the tryptophan is exposed or flexible, the peak is at 350 nm. For GLP-2 peptibody B, a ratio of between 0.8 to 0.85 was observed at room temperature for various dilutions of GLP-2 peptibody. The results are shown in FIG. 9E. From a sigmoid fit plot of the results shown in FIG. 9F, GLP-2 peptibody B has a Kd of 1043±154 nM. Also, the Kd for teduglutide is 77±14 μM with nanoDSF.

### Example 8: Mouse Pharmacokinetic Data for GLP-2 Peptibody K274

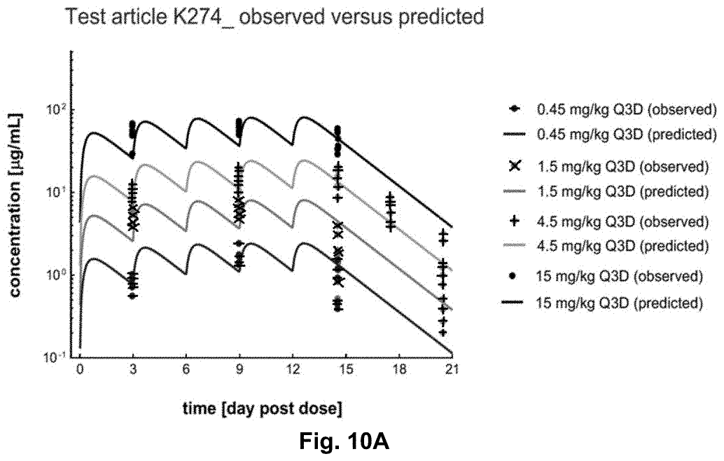

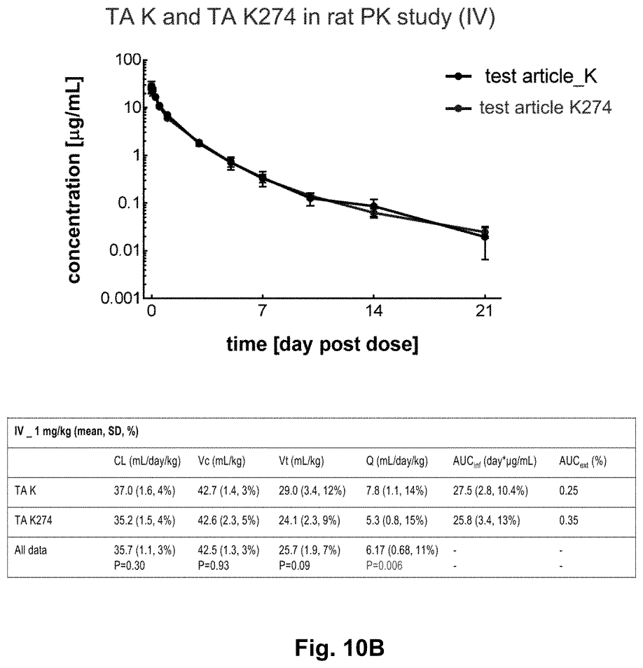

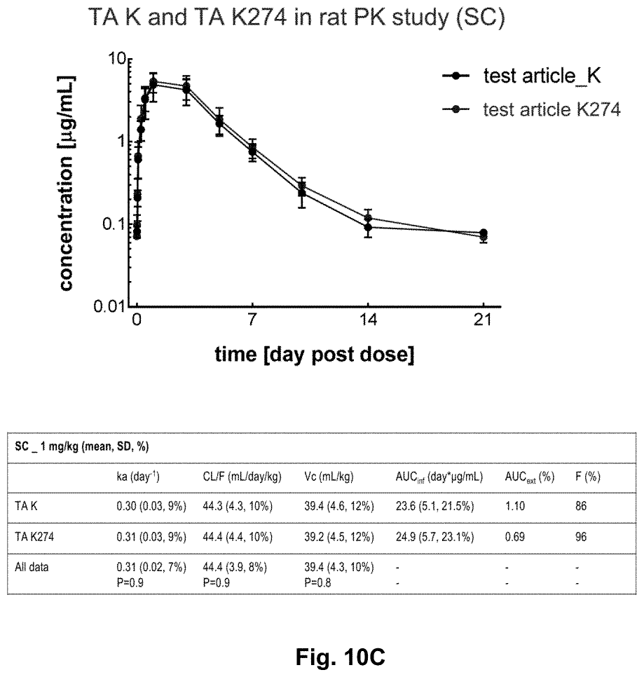

A pharmacokinetics analysis was performed in CD1 mice. The association constant (ka) is 3.04 day⁻¹, the CL/F is 81.3 mL/day/kg and the V_{c} is 213 mL/kg. Mice were divided into groups, with one group administered 0.45 mg/kg every three days (Q3D), another administered 1.5 mg/kg Q3D, another administered 4.5 mg/kg Q3D, and another administered 15 mg/kg Q3D over a 14 day period. After dosing was discontinued, concentrations were measured 3, 9, 14, and 21 days later. The results are shown in FIG. 10A.

### Example 9: Comparability of Pharmacokinetics of GLP-2 Peptibody K (with C-Terminal Lysine) and GLP-2 Peptibody K274 (without C-Terminal Lysine)

1 mg/kg of total GLP-2 peptibody K protein was administered subcutaneously to one group of six male Sprague-Dawley rats. 1 mg/kg of total GLP-2 peptibody K274 protein was administered intravenously to another group of six male Sprague-Dawley rats. 1 mg/kg of total GLP-2 peptibody B protein was administered subcutaneously to a third group of five male Sprague-Dawley rats. 1 mg/kg of total GLP-2 peptibody B264 protein was administered subcutaneously to a fourth group of five male Sprague-Dawley rats.

For all of the above groups, plasma samples were taken pre-dose, and at the following time points post-dose: 5 minutes (day 1), 10 minutes (day 1), 20 minutes (day 1), 30 minutes (day 1), 1 hour (day 1), 2 hours (day 1), 6 hours (day 1), 24 hours (day 2), 48 hours (day 3), 72 hours (day 4), 120 hours (day 6), 168 hours (day 8), 240 hours (day 11), and 336 hours (day 15).

Tables showing the pharmacokinetic data comparing intravenously administered GLP-2 peptibody K and GLP-2 peptibody K274 are in FIG. 10B. Tables showing the pharmacokinetic data comparing subcutaneously administered GLP-2 peptibody K and GL-2 peptibody K274 are in FIG. 10C. The data show that GLP-2 peptibody K and GLP-2 peptibody K274 are identical from a pharmacokinetic point of view.

### Example 10: Cynomolgus Monkey Pharmacokinetic Study with Teduglutide, GLP-2 Peptibody B, and GLP-2 Peptibody K

Pharmacokinetics studies of teduglutide, GLP-2 Peptibody B and GLP-2 Peptibody K were formed in cynomolgus monkeys with citrulline assayed as a biomarker of GLP-2 concentration. In the study, 12.5 nmol/kg teduglutide was administered subcutaneously to a group of 6 male cynomolgus monkeys at day 1. Then for one set of 2 monkeys, 25 nmol/kg GLP-2 Peptibody B was administered intravenously at day 7, day 21, day 28, day 35, and day 42. For another set of 3 monkeys, 25 nmol/kg GLP-2 Peptibody B was administered subcutaneously at day 7, day 21, day 28, day 35, and day 42. For another set of monkeys, 5 nmol/kg GLP-2 Peptibody K was administered intravenously (2 monkeys) and subcutaneously (3 monkeys) at day 7, day 21, day 28, day 35, and day 42. For another set of monkeys, 25 nmol/kg GLP-2 Peptibody K was administered subcutaneously (3 monkeys) and intraveneously (2 monkeys) at day 7, day 21, day 28, day 35, and day 42.

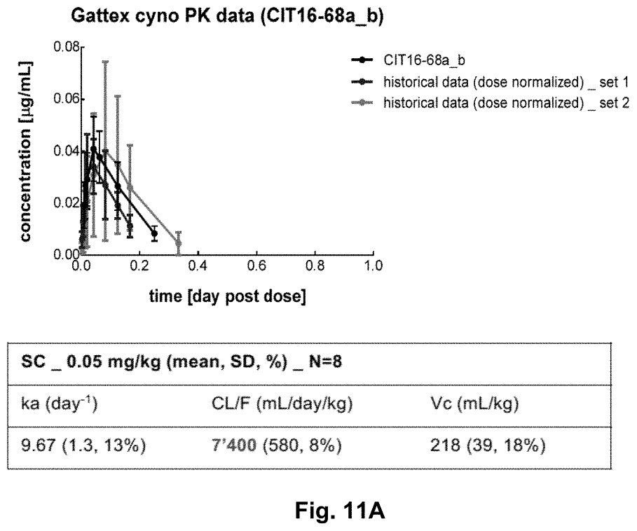

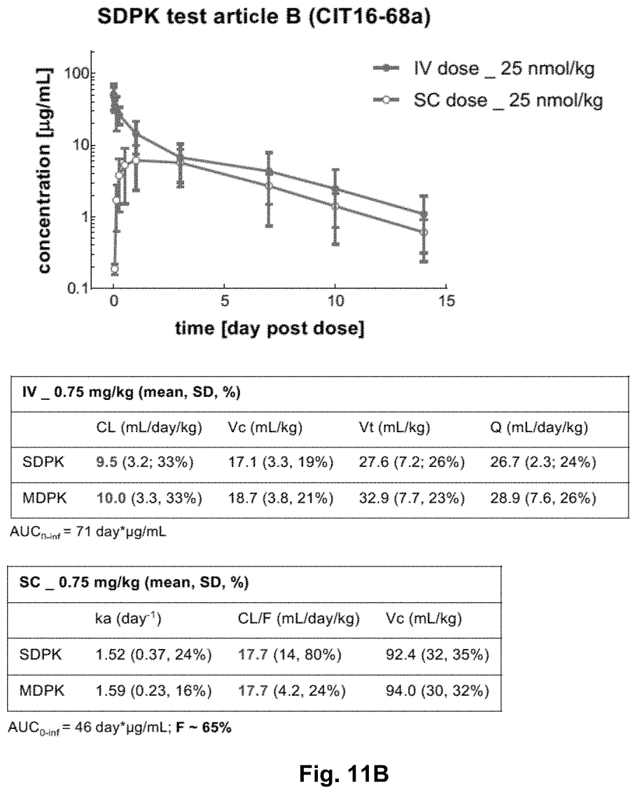

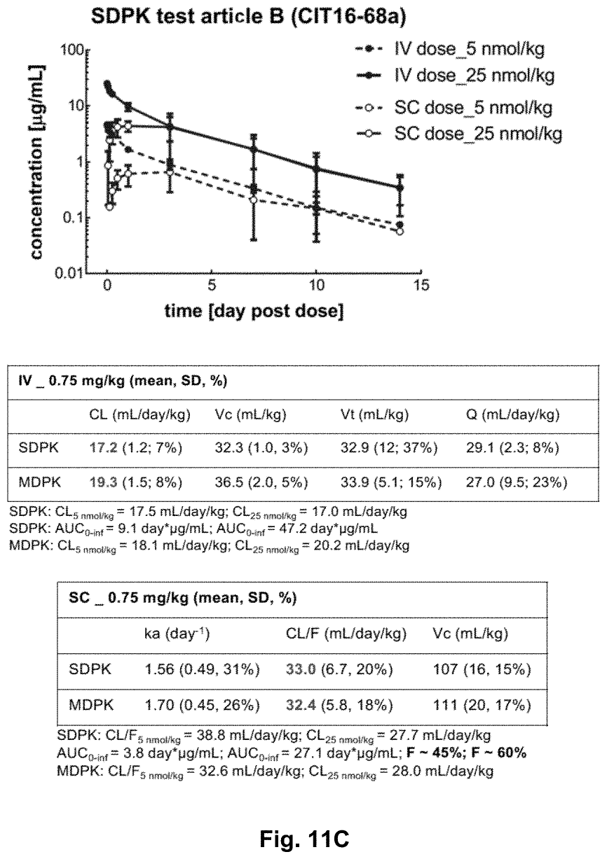

The results for subcutaneous teduglutide are shown in FIG. 11A. The association constant (ka) is 9.67 day⁻¹ (SD=1.3, 13%), the CL/F is 7,400 mL/day/kg (SD=580, 8%) and the V_{c} is 218 mL/kg (SD=39, 18%).

The results for intravenous and subcutaneous GLP-2 Peptibody B are shown in FIG. 11B. For single dose pharmacokinetics (SDPK) of an intravenous dose of 0.75 mg/kg, the CL is 9.5 mL/day/kg (SD=3.2, 33%), the V_{c} is 17.1 mL/kg (SD=3.3, 19%), the Vt is 27.6 mL/kg (SD=7.2, 26%), and the Q is 26.7 mL/day/kg (SD=2.3, 24%). For multiple dose pharmacokinetics (MDPK) of an intravenous dose of 0.75 mg/kg, the CL is 10.0 mL/day/kg (SD=3.3, 33%), the V_{c} is 18.7 mL/kg (SD=3.8, 21%), the Vₜ is 32.9 mL/kg (SD=7.7, 23%), and the Q is 28.9 mL/day/kg (SD=7.6, 26%). For SDPK (subcutaneous, 0.75 mg/kg), the association constant (ka) is 1.52 day⁻¹ (SD=0.37, 24%), the CL/F is 17.7 mL/day/kg (SD=14, 80%) and the V_{c} is 92.4 mL/kg (SD=32, 35%). For MDPK (subcutaneous, 0.75 mg/kg), the association constant (ka) is 1.59 day′ (SD=0.23, 16%), the CL/F is 17.7 mL/day/kg (SD=4.2, 24%) and the V_{c} is 94.0 mL/kg (SD=30, 32%).

The results for intravenous and subcutaneous GLP Peptibody K are shown in FIG. 11C. For SDPK (intravenous, 0.75 mg/kg), the CL is 17.2 mL/day/kg (SD=1.2, 7%), the V_{c} is 32.3 mL/kg (SD=1.0, 3%), the Vₜ is 32.9 mL/kg (SD=12, 37%), and the Q is 29.1 mL/day/kg (SD=2.3, 8%). For MDPK (intravenous, 0.75 mg/kg), the CL is 19.3 mL/day/kg (SD=1.5, 8%), the V_{c} is 36.5 mL/kg (SD=2.0, 5%), the Vₜ is 33.9 mL/kg (SD=5.1, 15%), and the Q is 27.0 mL/day/kg (SD=9.5, 23%). For SDPK (subcutaneous, 0.75 mg/kg), the association constant (ka) is 1.56 day⁻¹ (SD=0.49, 31%), the CL/F is 33.0 mL/day/kg (SD=6.7, 20%) and the V_{c} is 107 mL/kg (SD=16, 15%). For MDPK (subcutaneous, 0.75 mg/kg), the association constant (ka) is 1.70 day⁻¹ (SD=0.45, 26%), the CL/F is 32.4 mL/day/kg (SD=5.8, 18%) and the V_{c} is 111 mL/kg (SD=20, 17%).

While a dose of 30 μg/kg once weekly (QW) is projected from the cynomolgus monkey PK data, the dose should be ten times higher (300 μg/kg) to adjust for the difference in in vivo potency. The following table shows the projection for intravenous and subcutaneous parameters for humans, with the exponent on the CL equal to 0.85, the cynomolgus monkey body weight equal to 3.5 kg, and the human body weight equal to 70 kg.

TABLE 8

| | | CL | Vc | Vt | Q | |
|---|---|---|---|---|---|---|
| Com- | ka | (mL/ | (mL/ | (mL/ | (mL/ | F |
| pound | (day⁻¹) | day/kg) | kg) | kg) | day/kg) | (%) |
| GLP-2 | 2.43 | 39.2 (25.0) | 49.4 | 42.5 | 24.1 (15.4) | 98 (60) |
| Peptibody | | | | | | |
| B | | | | | | |
| GLP-2 | 1.40 | 24.2 (15.4) | 38.5 | 36.1 | 56.4 (36.0) | 86 (60) |
| Peptibody | | | | | | |
| K | | | | | | |

For a 1.5 mL subcutaneous injection, the concentration would be 15 mg/mL. For a 2.0 mL subcutaneous injection, the concentration would be 10 mg/mL.

### Example 11: Pharmacodynamic Plateau Study with GLP-2 Peptibody K274

Various doses of GLP-2 peptibody K274 were analyzed in female CD-1 mice to assess the pharmacodynamic plateau, with the primary endpoint a measurement of the small intestinal weight relative to the total body weight and a histology study of the length of villi. Eight groups of six females each were formed. In two groups, only the vehicle was administered Q3D for as a negative control. In four groups, the following doses were administered Q3D over 14 days: 0.45 mg/kg, 1.5 mg/kg, 4.5 mg/kg and 15 mg/kg. In one additional group, 4.5 mg/kg was administered Q3D for 14 days with the study ending four days later at day 18. In another additional group, 4.5 mg/kg was administered Q3D for 14 days with the study ending seven days later at day 21. The groups are summarized in Table 9 below.

TABLE 9

| | | Dose | Dose | Study |
|---|---|---|---|---|
| Group | Test agent | (mg/kg) | Frequency | Duration |
| 15 | Vehicle 1 | n/a | Q3D | 14 days |
| 16 | Vehicle 2 | n/a | Q3D | 21 days |
| 17 | GLP-2 peptibody K274 | 0.45 | Q3D | 14 days |
| 18 | GLP-2 peptibody K274 | 1.5 | Q3D | 14 days |
| 19 | GLP-2 peptibody K274 | 4.5 | Q3D | 14 days |
| 20 | GLP-2 peptibody K274 | 15 | Q3D | 14 days |
| 21 | GLP-2 peptibody K274 | 4.5 | Q3D over 14 | 18 days |
| | | | days | |
| 22 | GLP-2 peptibody K274 | 4.5 | Q3D over 14 | 21 days |
| | | | days | |

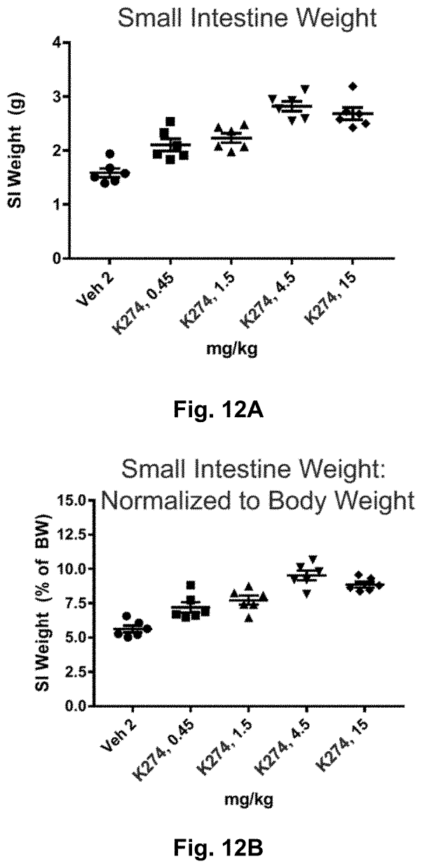

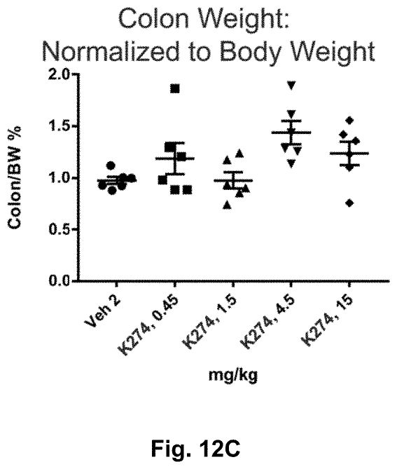

For the primary endpoint, the small intestine weight in grams is shown in FIG. 12A, the small intestine weight normalized to body weight is shown in FIG. 12B, and the colon weight normalized to body weight is shown in FIG. 12C. A dose of 4.5 mg/kg had maximum effect.

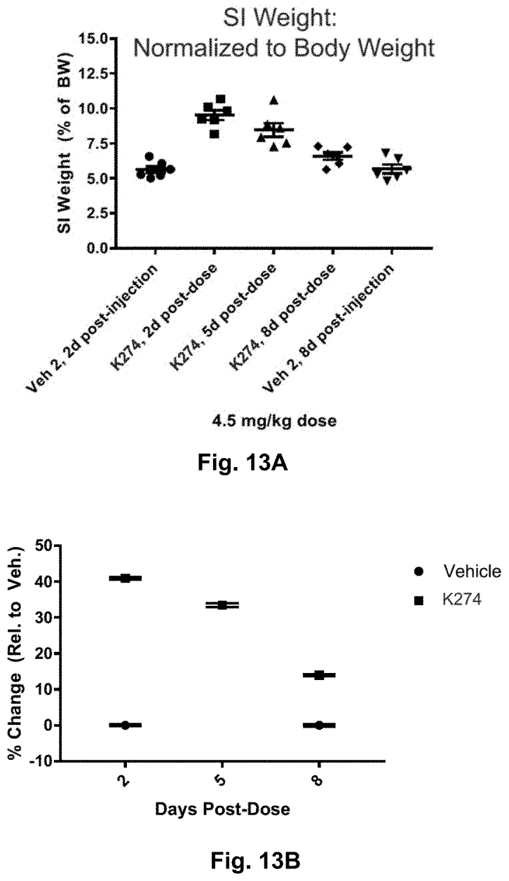

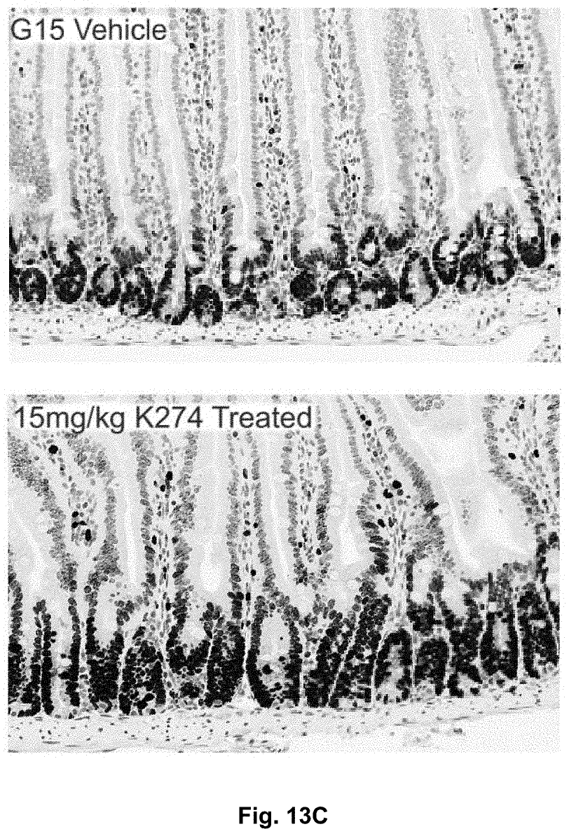

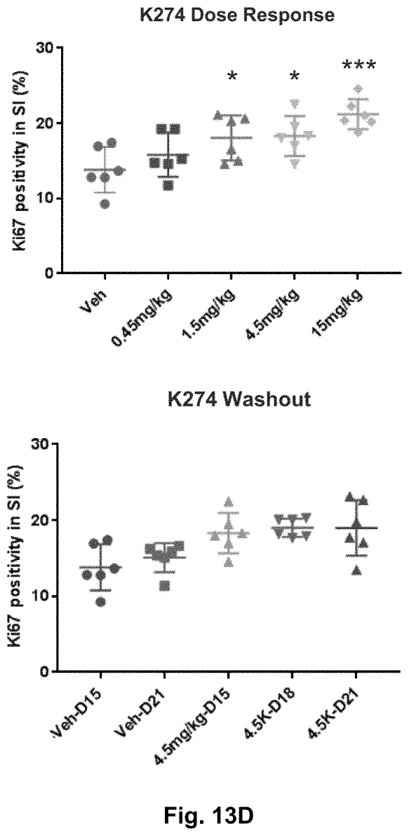

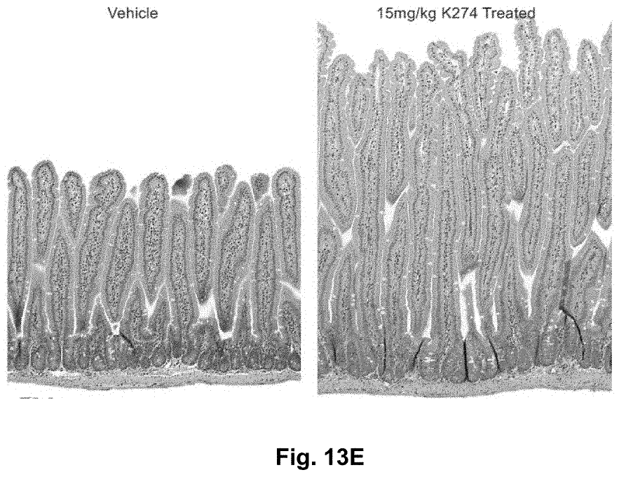

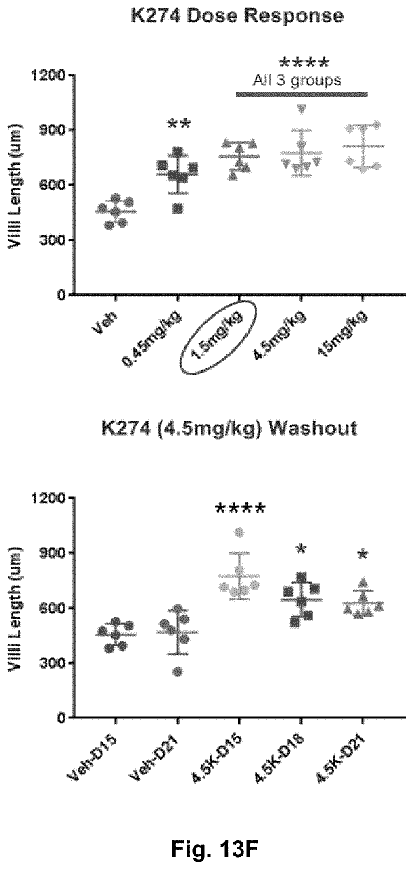

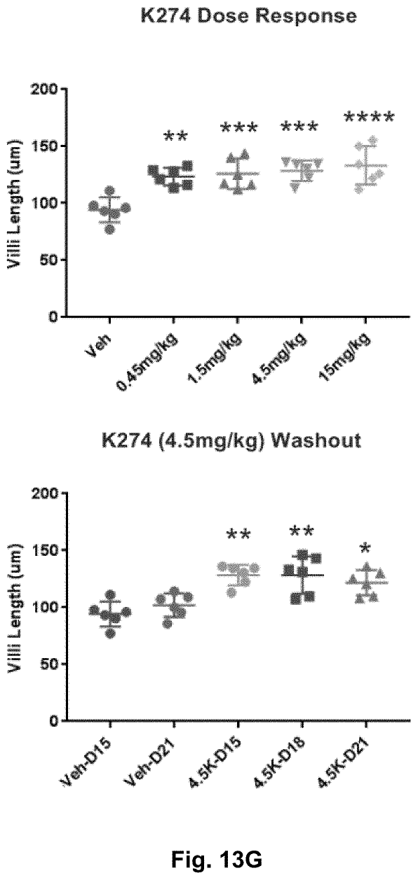

Further, an effect on increased small intestine weight normalized to body weight persisted for at least five days after dosing, as shown in FIG. 13A. FIG. 13B is a graph depicting the percentage change in small intestine weight for both vehicle and GLP-2 peptibody K274.

For the histology study, 4 micron paraffin sections were prepared for H&E and Ki67 staining. After whole slide scanning, an imagescope was used to take villi length measurements, crypt depth measurements, and Ki67 analysis. The Ki67 staining results are shown in FIG. 13C. The results of a dose-response study and a washout study with Ki67 percent positivity are shown in FIG. 13D.

A histology slide showing villi length in vehicle-treated and 15 mg/kg GLP-2 peptibody K274 treated (Q3D over 14 days) is depicted in FIG. 13E. The villi length in microns was measured for the different groups above, with results shown in FIG. 13F. The crypt depth in microns was measured for the different groups above, with results shown in FIG. 13G.

### Example 12: Pharmacodynamic Plateau Study with GLP-2[A2G]

GLP-2[A2G] peptide was analyzed in a histology study in CD-1 mice to assess the length of villi and crypt depth. The GLP-2[A2G] peptide used in this study was prepared using a peptide synthesizer. Eight groups of six females each were formed. In two groups, only the vehicle was administered twice a day (BID) for as a negative control. In six groups, the following doses were administered BID over 15 days: 0.0125 mg/kg, 0.025 mg/kg, 0.050 mg/kg, 0.100 mg/kg, 0.250 mg/kg, and 0.500 mg/kg. In one additional group, 0.500 mg/kg was administered BID for 14 days with the study ending two days later at day 16. In another additional group, 0.500 mg/kg was administered BID for 14 days with the study ending two days later at day 18. In yet another additional group, 0.500 mg/kg was administered BID for 10 days with the study ending two days later at day 21. The groups are summarized in Table 10 below.

TABLE 10

| | | Dose | Dose | Study |
|---|---|---|---|---|
| Group | Test agent | (mg/kg) | Frequency | Duration |
| 1 | Vehicle 1 | n/a | BID | 15 days |
| 2 | Vehicle 2 | n/a | BID | 21 days |
| 3 | GLP-2[A2G] | 0.0125 | BID | 15 days |
| 4 | GLP-2[A2G] | 0.025 | BID | 15 days |
| 5 | GLP-2[A2G] | 0.050 | BID | 15 days |
| 6 | GLP-2[A2G] | 0.100 | BID | 15 days |
| 7 | GLP-2[A2G] | 0.250 | BID | 15 days |
| 8 | GLP-2[A2G] | 0.500 | BID | 15 days |
| 9 | GLP-2[A2G] | 0.500 | BID over 14 | 16 days |
| | | | days | |
| 10 | GLP-2[A2G] | 0.500 | BID over 14 | 18 days |
| | | | days | |
| 11 | GLP-2[A2G] | 0.500 | BID over 14 | 21 days |
| | | | days | |

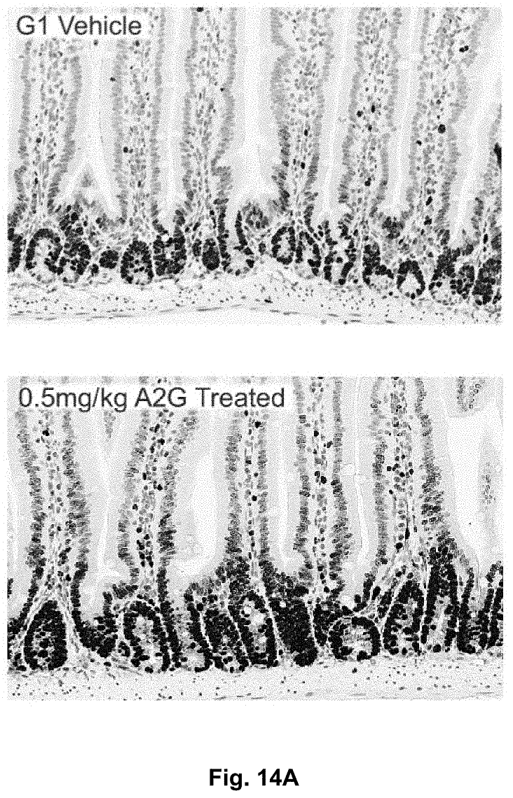

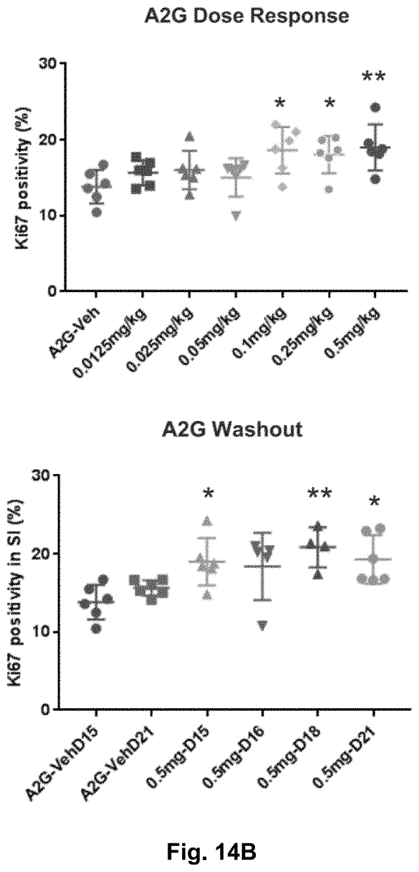

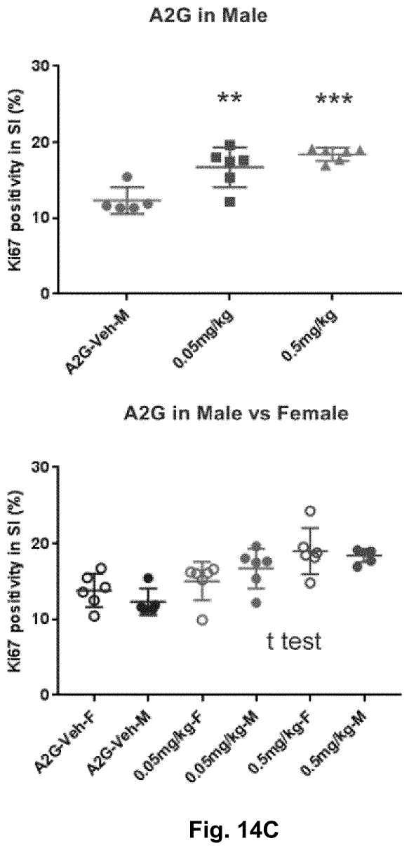

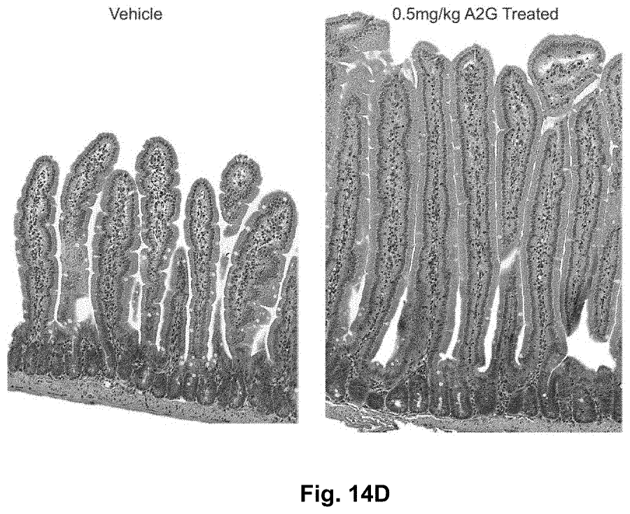

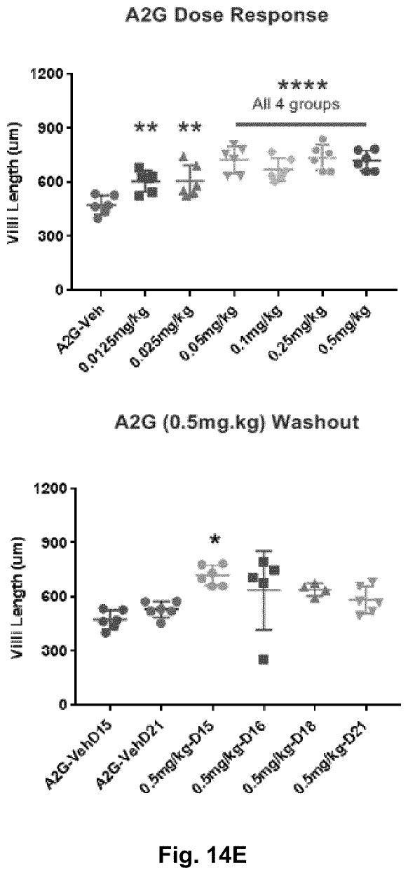

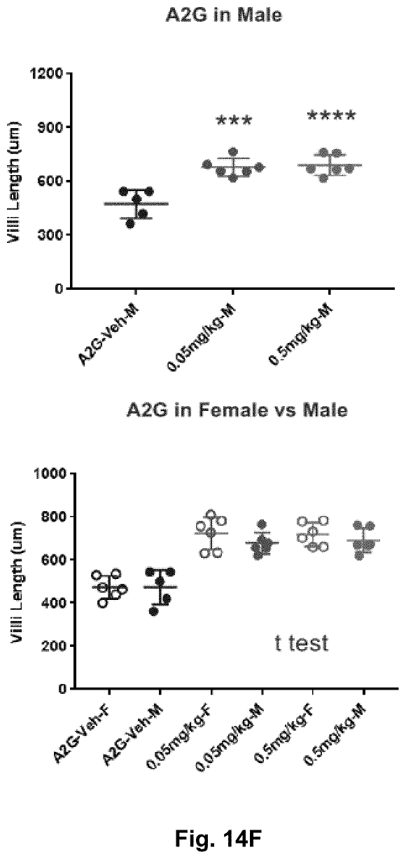

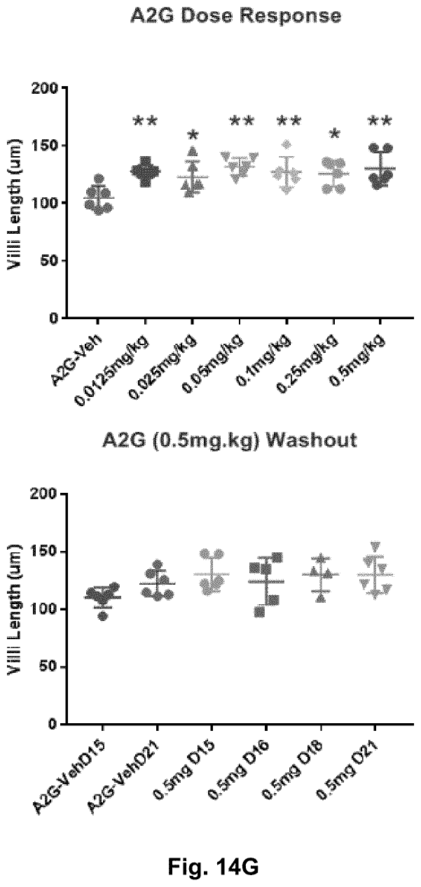

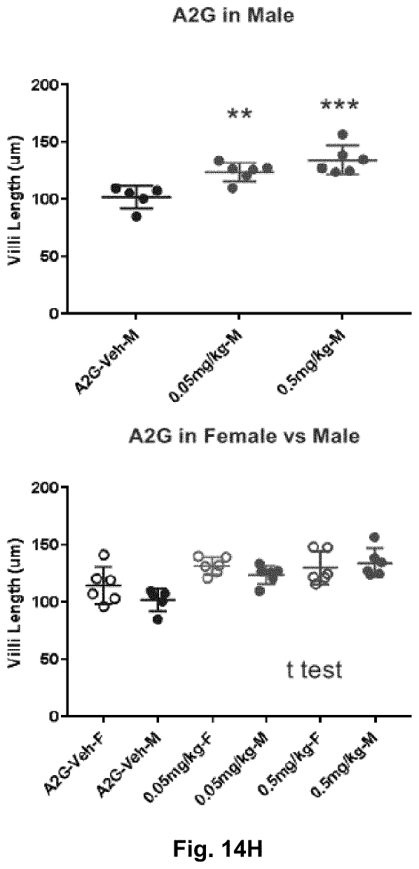

For the histology study, 4 micron paraffin sections were prepared for H&E and Ki67 staining. After whole slide scanning, an imagescope was used to take villi length measurements, crypt depth measurements, and Ki67 analysis. The results of the Ki67 staining are shown in FIG. 14A. The results of a dose-response study with Ki67 percent positivity are shown in FIG. 14B.

FIG. 14C shows the extent of Ki67 positivity in males administered doses of vehicle, 0.05 mg/kg GLP-2[A2G] and 0.5 mg/kg GLP-2[A2G] BID over 15 days, along with a comparison between males and females administered the same over 15 days.

A histology slide showing villi length in vehicle-treated and 0.5 mg/kg GLP-2[A2G] treated (BID over 14 days) is depicted in FIG. 14D. The villi length in microns was measured for the different groups above, with results shown in FIG. 14E. FIG. 14F shows the villi length in males administered doses of vehicle, 0.05 mg/kg GLP-2[A2G] and 0.5 mg/kg GLP-2[A2G] BID over 15 days, along with a comparison between males and females administered the same over 15 days.

The crypt depth in microns was measured for the different groups above, with results shown in FIG. 14G. FIG. 14H shows the crypt depth in males administered doses of vehicle, 0.05 mg/kg GLP-2[A2G] and 0.5 mg/kg GLP-2[A2G] BID over 15 days, along with a comparison between males and females administered the same over 15 days.

### Example 13: Dose-Response Study with GLP-2[A2G], GLP Peptibody B264 and GLP Peptibody K274

Various doses of GLP-2[A2G] peptide prepared using a peptide synthesizer were analyzed to assess pharmacokinetics and pharmacodynamics, with the primary endpoint a measurement of the absolute small intestinal weight, in grams, and relative small intestinal weight as a percentage of the total body weight. Three groups of six females each were formed, as shown in Table 11 below:

TABLE 11

| | | Dose | Dose | Study |
|---|---|---|---|---|
| Group | Test agent | (mg/kg/day) | Frequency | Duration |
| 1 | Vehicle 1 | n/a | BID | 14 days |
| 2 | GLP-2[A2G] | 0.050 | BID | 14 days |
| 3 | GLP-2[A2G] | 0.500 | BID | 14 days |

Various doses of GLP-2 peptibody B264 were analyzed to assess pharmacokinetics and pharmacodynamics, with the primary endpoint a measurement of the absolute small intestinal weight, in grams, and relative small intestinal weight as a percentage of the total body weight. Eight groups of six female CD-1 mice each were formed. In two groups, only the vehicle was administered every three days (Q3D) as a negative control. The study duration was 14 days for one of these groups and 21 days for the other group. In four additional groups, the following doses were administered Q3D over 14 days: 0.45 mg/kg, 1.5 mg/kg, 4.5 mg/kg, 15 mg/kg. In one more group, 4.5 mg/kg was administered Q3D for 14 days, with the study duration of 18 days. In one more group, 4.5 mg/kg was administered Q3D for 14 days, with the study duration of 21 days. All of these groups are summarized in Table 12 below.

TABLE 12

| | | Dose | Dose | Study |
|---|---|---|---|---|
| Group | Test agent | (mg/kg) | Frequency | Duration |
| 1 | Vehicle 1 | n/a | 1x every 3 days | 14 days |
| 2 | Vehicle 2 | n/a | 1x every 3 days | 21 days |
| 3 | GLP peptibody B264 | 0.45 | 1x every 3 days | 14 days |
| 4 | GLP peptibody B264 | 1.5 | 1x every 3 days | 14 days |
| 5 | GLP peptibody B264 | 4.5 | 1x every 3 days | 14 days |
| 6 | GLP peptibody B264 | 15 | 1x every 3 days | 14 days |
| 7 | GLP peptibody B264 | 4.5 | 1x every 3 days, | 18 days |
| | | | for 14 days only | |
| 8 | GLP peptibody B264 | 4.5 | 1x every 3 days, | 21 days |
| | | | for 14 days only | |

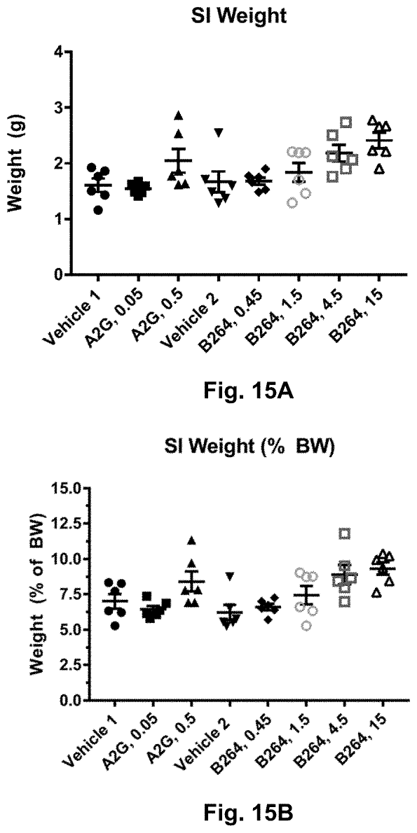

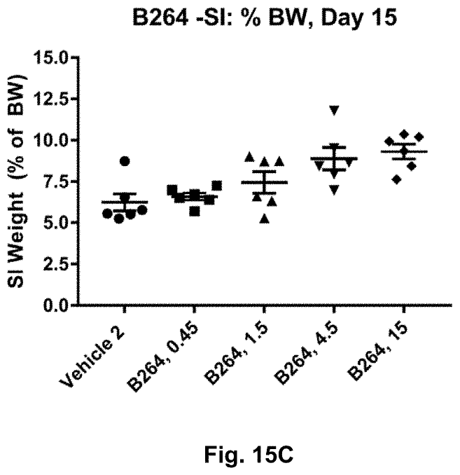

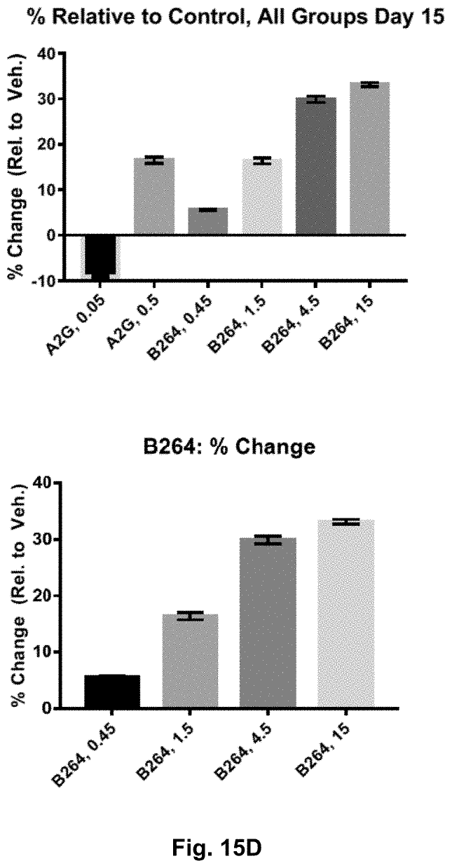

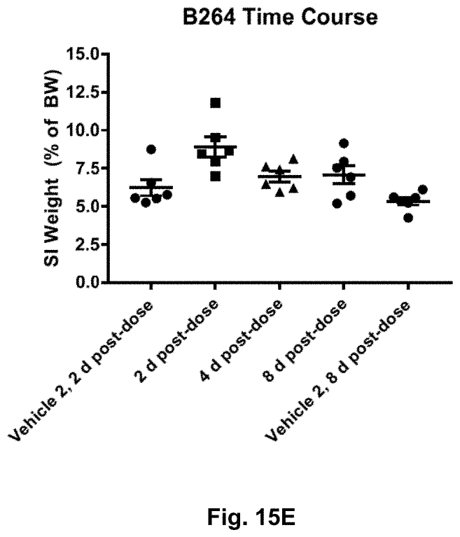

For the primary endpoint for the above GLP-2[A2G] and GLP-2 Peptibody B264 groups, the small intestine weight in grams is shown in FIG. 15A and the small intestine weight normalized to body weight is shown in FIG. 15B. At the 15 days time point, FIG. 15C shows the small intestine weight as a percentage of body weight. On the X axis, the doses are listed in mg/kg.

FIG. 15D is a graph showing the percentage change in gut weight relative to the control at day 15.

For the above groups 1, 2, 5, 7 and 8, an assay of the small intestine weight as compared to total body weight was undertaken. The results are shown in FIG. 15E. In FIG. 15E with respect to GLP-2 peptibody B264, “Vehicle 2, 2 d post-dose” corresponds to group 1 at day 14, “2 d post-dose” corresponds to group 5 at day 14, “4 d post-dose” corresponds to group 7 at day 18, “8 d post-dose” corresponds to group 8 at day 20, and “vehicle 2, 8 d post-dose” corresponds to group 2 at day 20.

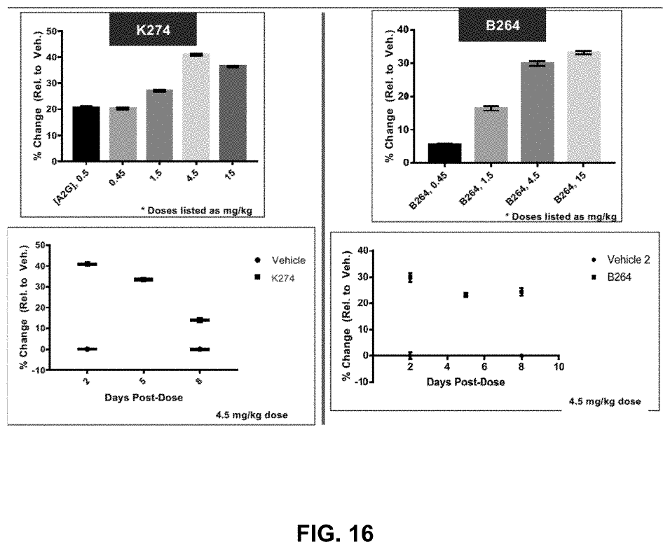

FIG. 16 summarizes the relative change in small intestinal weight for both GLP-2 peptibody K274 and GLP-2 peptibody B264, relative to control and washout.

### Example 14: Histology Study of Villi Length and Crypt Depth in GLP-2 Peptibody B264

Various doses of GLP-2 peptibody B264 were analyzed to assess the pharmacodynamic plateau, with the primary endpoint a measurement of the small intestinal weight relative to the total body weight and a histology study of the length of villi. 11 groups of six female CD-1 mice each were formed. The groups are summarized in Table 13 below.

TABLE 13

| | | Dose | Dose | Study |
|---|---|---|---|---|
| Group | Test agent | (mg/kg) | Regimen | Duration |
| 1 | Vehicle 1 | 0 | BID, 14 days | 15 days |
| 2 | GLP-2[A2G] | 0.025 | Q3D, 14 | 15 days |
| | | | days | |
| 3 | GLP-2[A2G] | 0.25 | Q3D, 14 | 15 days |
| | | | days | |
| 4 | Vehicle 2 | 0 | Q3D, 14 | 15 days |
| | | | days | |
| 5 | Vehicle 2 | 0 | Q3D, 14 | 21 days |
| | | | days | |
| 6 | GLP-2 peptibody | 0.45 | Q3D | 15 days |
| | B264 | | | |
| 7 | GLP-2 peptibody | 1.5 | Q3D over 14 | 15 days |
| | B264 | | days | |
| 8 | GLP-2 peptibody | 4.5 | Q3D over 14 | 15 days |
| | B264 | | days | |
| 9 | GLP-2 peptibody | 15 | Q3D over 14 | 15 days |
| | B264 | | days | |
| 10 | GLP-2 peptibody | 4.5 | Q3D over 14 | 18 days |
| | B264 | | days | |
| 11 | GLP-2 peptibody | 4.5 | Q3D over 14 | 21 days |
| | B264 | | days | |

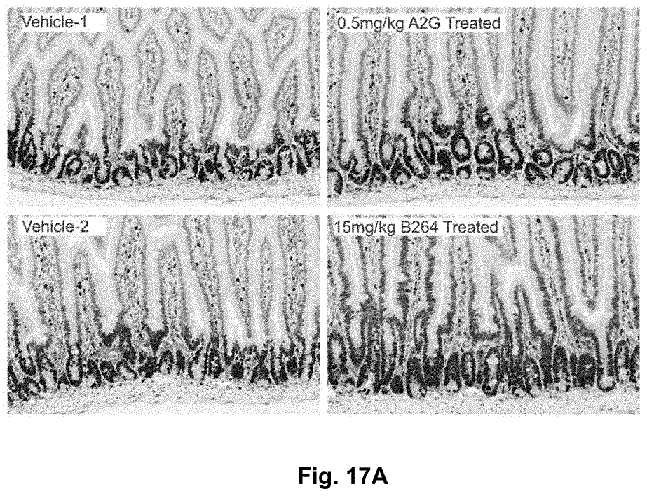

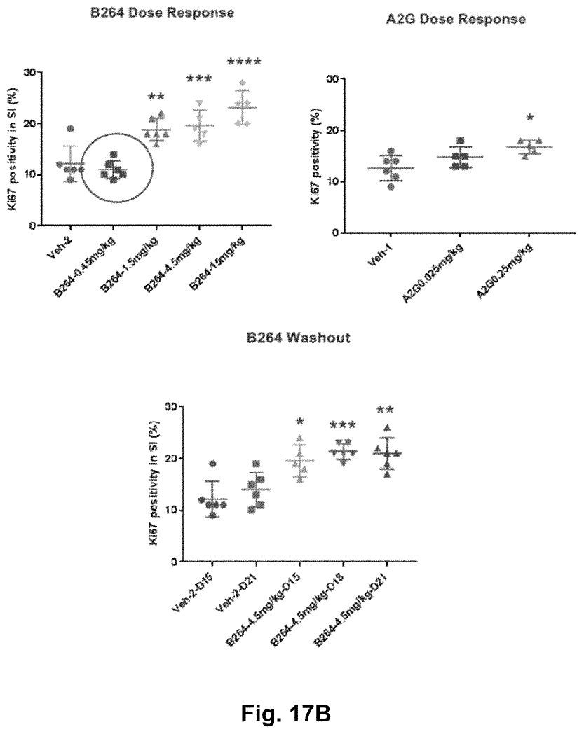

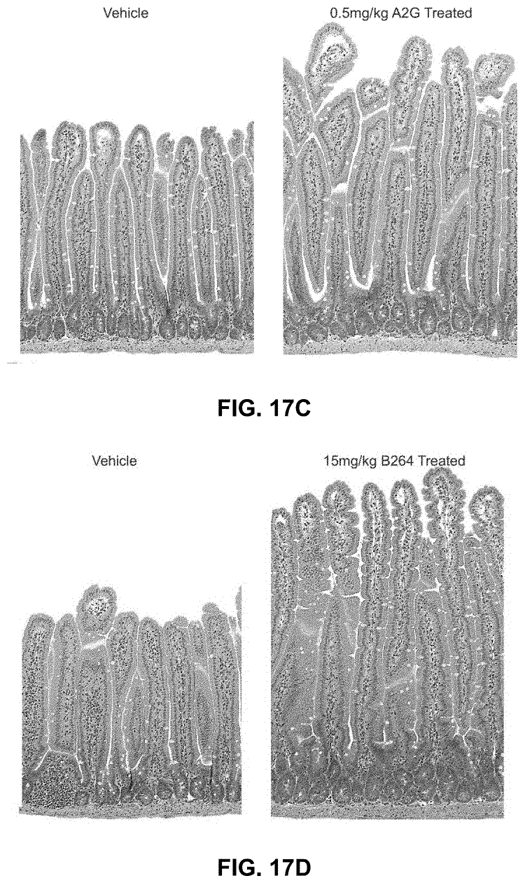

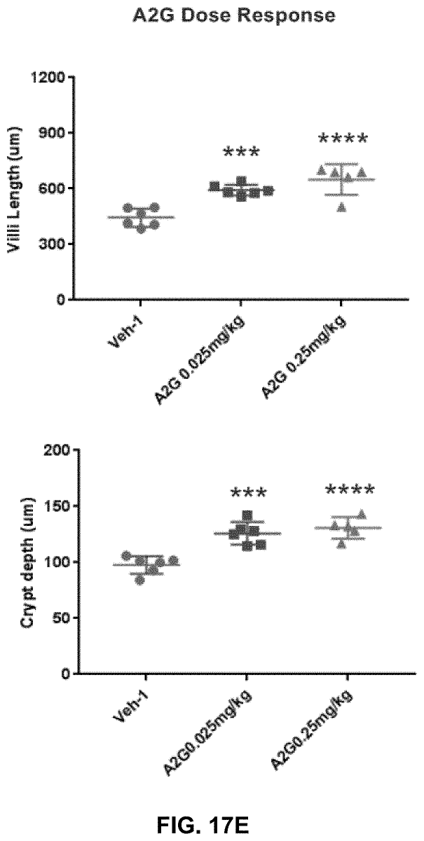

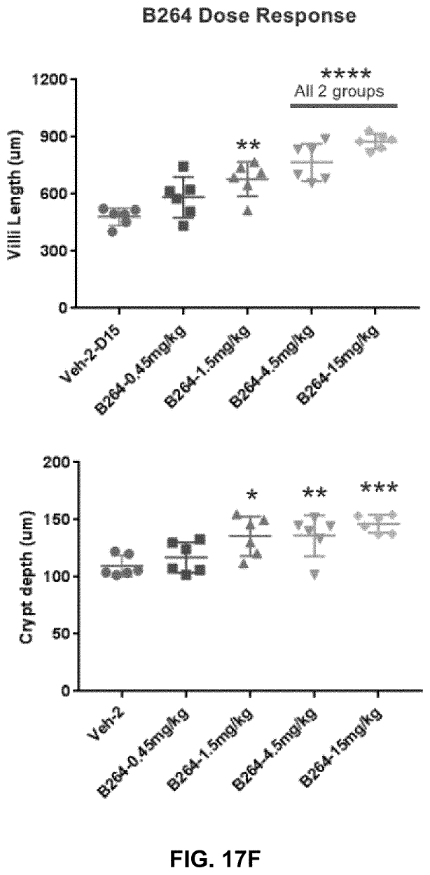

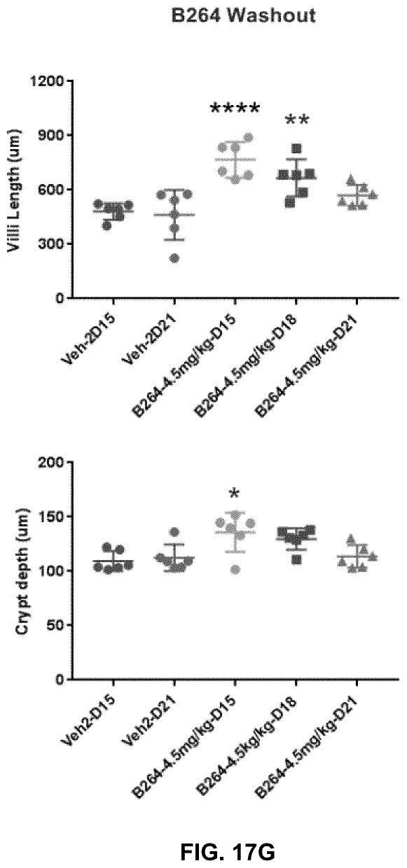

For histology, four micron paraffin sections were prepared for H&E and Ki67 IHC staining. After whole slide scanning, an imagescope was used to measure villi length and crypt depth, and to analyze Ki67. The antibody against Ki67 is a rabbit antibody sold by Adcam®, catalog number ab 616667. The antibody was used at a working concentration of 1:100 and was detected using a Leica® Refine Kit. The Ki67 staining results are shown in FIG. 17A. The results of a dose-response study and a washout study with Ki67 percent positivity are shown in FIG. 17B.

A comparison between vehicle and 0.5 mg/kg/day GLP-2[A2G] treated groups is shown in FIG. 17C. A comparison between vehicle and 15 mg/kg GLP-2 peptibody B264 treated groups is shown in FIG. 17D. The villi length in microns was measured for groups 1 and 2 above (GLP-2[A2G]), with results shown in FIG. 17E. The villi length in microns was measured for groups 1-3 above (vehicle and GLP-2[A2G]), with results shown in FIG. 17E. The villi length in microns was measured for groups 4 and 6-9 above (vehicle and GLP-2 peptibody B264), with results shown in FIG. 17F. The villi length in microns was measured for groups 4, 5 and 9-11 above (vehicle and GLP-2 peptibody B264), with results shown in FIG. 17G.

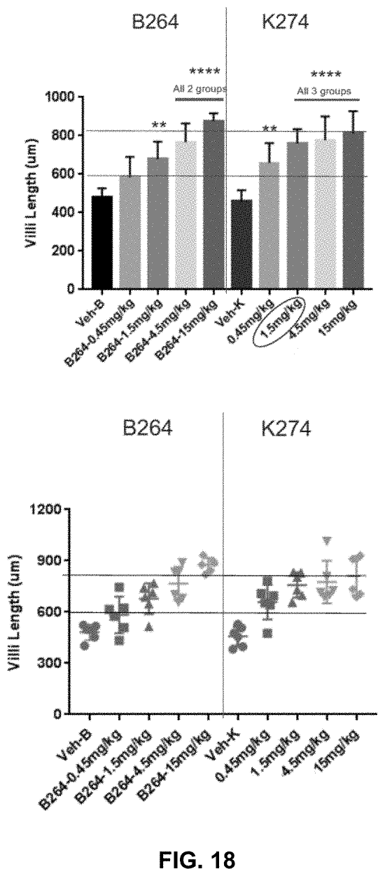

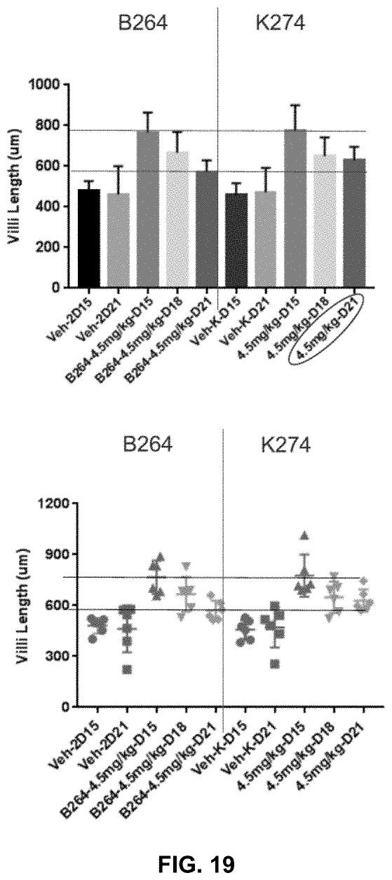

A comparison of villi length between GLP-2 peptibody B264 and GLP-2 peptibody K274 is shown in FIG. 18 at various doses. FIG. 19 shows a comparison of villi length between 4.5 mg/kg GLP-2 peptibody B264 and 4.5 mg/kg GLP-2 peptibody K274 at various time points during a washout period after the Q3D dosage regimen over 14 days ends. The first day after the washout period ends is day 15, the second day is day 16, etc. Day 2 of the washout period corresponds with day 15. Day 5 of the washout period corresponds with day 18. Day 8 of the washout period corresponds with day 21. D15, D18, and D21 correspond to days 15, 18 and 21 on which the villi length was measured.

### Example 15: Summary of Mouse Pharmacokinetics and Pharmacodynamics Test Data

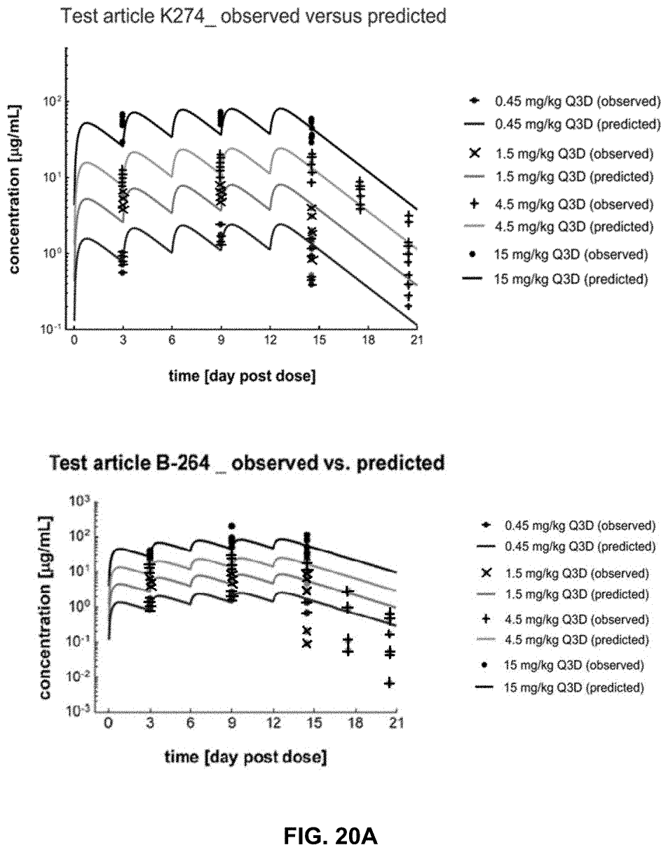

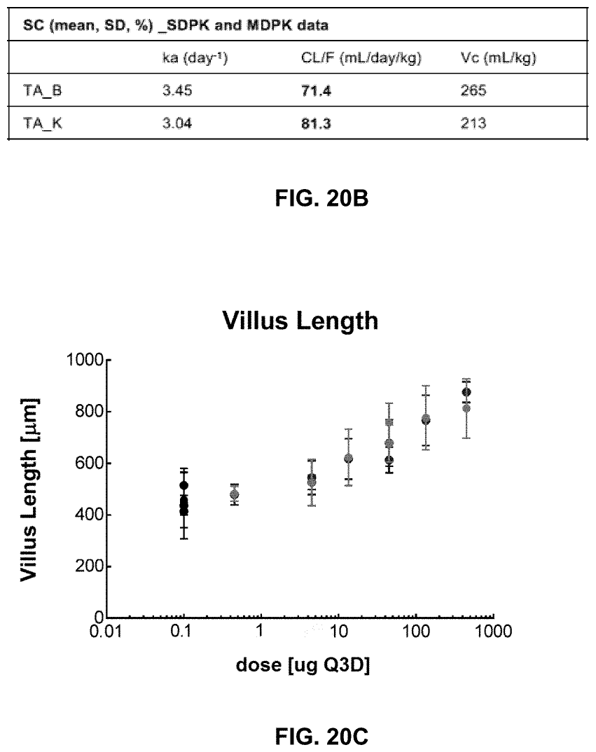

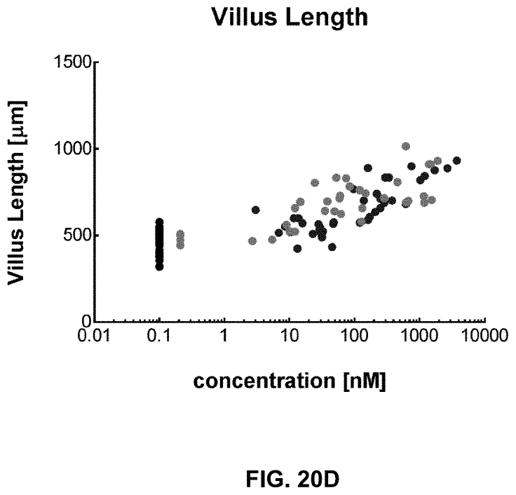

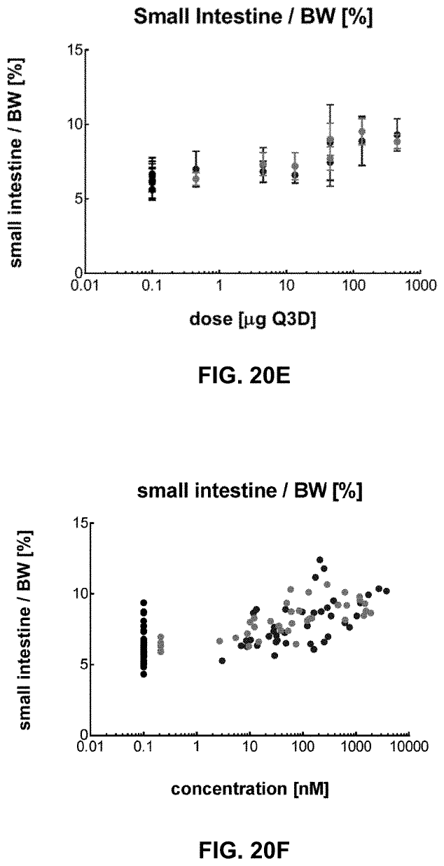

FIG. 20A shows a comparison between the GLP-2 peptibody B264 and GLP-2 peptibody K274 concentration over a 14 day Q3D dosing regimen. The solid line is the predicted concentration and the dots represent various observed concentrations.

FIG. 20B shows a summary of pharmacokinetics data on GLP-2 peptibody B264 and GLP-2 peptibody K274 in the mouse.

FIG. 20C shows a comparison of villus length between GLP-2 peptibody B264 and GLP-2 peptibody K274 at various doses. FIG. 20D shows a comparison of villus length between GLP-2 peptibody B264 and GLP-2 peptibody K274 at various concentrations.

FIG. 20E shows a comparison between GLP-2 peptibody B264 and GLP-2 peptibody K274 at various doses, with the primary endpoint of small intestine weight as a percentage of body weight. FIG. 20F shows a comparison between GLP-2 peptibody B264 and GLP-2 peptibody K274 at various concentrations, with the primary endpoint of small intestine weight as a percentage of body weight.

### Example 16: GLP-2 Peptibody K274 Enhances Dietary Fat Absorption

A fat tolerance assay was performed in mice to assess the ability of GLP-2 peptibody K274 to promote absorption of dietary fats. Dietary fat is hydrolyzed into free fatty acids and glycerides, which are transported through the intestinal villi and absorbed by enterocytes. The enterocytes synthesize the triglycerides, which then enter the bloodstream. Such postprandial triglycerides peak in the bloodstream at about 3 hours after ingestion of a fat-rich meal.

It is hypothesized that GLP-2 peptibody K274, by enhancing length of the intestinal villi, would improve the absorption of fatty acids in a mouse model of short bowel syndrome. Assaying for an increase in peak postprandial triglycerides allows for detection of such increased absorption.

Female mice were divided into two groups of 30 mice each. Both groups were treated every 3 days for a total of 13 days either with 4.5 mg/kg K274 peptibody (treated group) or vehicle (control group). On day 14 after start of treatment, mice in both groups were fasted for 6 hours followed by administration of an olive oil bolus of 10 mL/kg. Mice in the treated and control groups were divided into 6 subgroups of 6 animals each. A 100 μL blood sample was taken from each of the 6 mice per subgroup after 0 min, 15 min, 30 min, 1 hour, 2 hours, or 3 hours respectively. The blood was collected into K2EDTA tubes and centrifuged to obtain plasma. Plasma triglyceride concentrations were measured on a Cobas C311 instrument (Roche) using the TRIGB assay kit.

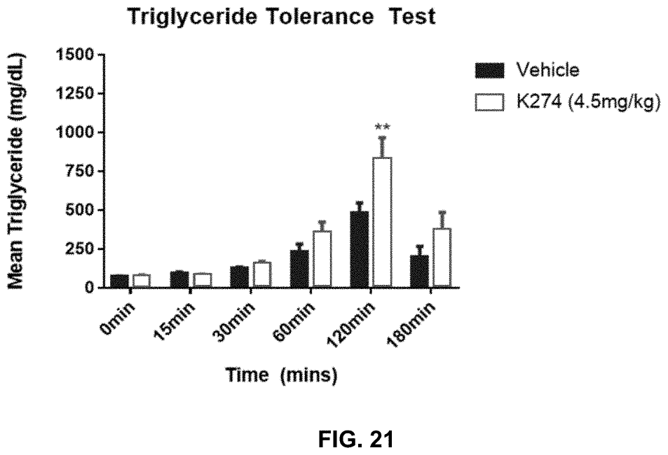

The data are shown in FIG. 21. The postprandial triglyceride concentration in the bloodstream was significantly higher in the mice treated with GLP-2 peptibody K274, indicating that GLP-2 peptibody K274 improves absorption of fatty acids.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

Patents, patent applications, publications, product descriptions, and protocols are cited throughout this application, the disclosures of which are incorporated herein by reference in their entireties for all purposes.

1.-106. (canceled)

107. A glucagon-like peptide (GLP-2) peptibody selected from the group consisting of:
a) a GLP-2 peptibody comprising the amino acid sequence of
(SEQ ID NO: 1)
HGDGSFSDEMNTILDNLAARDFINWLIQTKITDGGGGGDKTHTCPPCPA
PEAAGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSV
MHEALHNHYTQKSLSLSPG,
b) a GLP-2 peptibody comprising the amino acid sequence of
(SEQ ID NO: 4)
HGDGSFSDEMNTILDNLAARDFINWLIQTKITDGGGGGDKTHTCPPCPA
PEAAGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALP
APIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSV
MHEALHNHYTQKSLSLSPGK,
c) a GLP-2 peptibody comprising the amino acid sequence of
(SEQ ID NO: 7)
HGDGSFSDEMNTILDNLAARDFINWLIQTKITDGGGGSGGGGSGGGGSD
KTHTCPPCPAPEAAGGPSVFLFPPKPKDTLMISRTPEVTCVVVDVSHED
PEVKFNWYVDGVEVHNAKTKPREEQYNSTYRVVSVLTVLHQDWLNGKEY
KCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRW
QQGNVFSCSVMHEALHNHYTQKSLSLSPG,
d) a GLP-2 peptibody comprising the amino acid sequence of
(SEQ ID NO: 10)
GDGSFSDEMNTILDNLAARDFINWLIQTKITDGGGGSGGGGSGGGGSDK
THTCPPCPAPEAAGGPSVFLFPPKPKDTLMISRTPEVTCVVVDVSHEDP
EVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQ
QGNVFSCSVMHEALHNHYTQKSLSLSPGK,
e) a GLP-2 peptibody comprising the amino acid sequence of
(SEQ ID NO: 13)
HGDGSFSDEMNTILDNLAARDFINWLIQTKITDDKTHTCPPCPAPEAAG
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEAL
HNHYTQKSLSLSPG,
f) a GLP-2 peptibody comprising the amino acid sequence of
(SEQ ID NO: 16)
HGDGSFSDEMNTILDNLAARDFINWLIQTKITDGGGGGGSGGGGSGGGG
SDKTHTCPPCPAPEAAGGPSVFLFPPKPKDTLMISRTPEVTCVVVDVSH
EDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLT
CLVKGFYPSDIAVEWESNGQPENNYKTTPPVLDSDGSFFLYSKLTVDKS
RWQQGNVFSCSVMHEALHNHYTQKSLSLSPG,
g) a GLP-2 peptibody comprising the amino acid sequence of
(SEQ ID NO: 19)
HGDGSFSDEMNTILDNLAARDFINWLIQTKITDGAPGGGGGAAAAAGGG
GGGAPGGGGGAAAAAGGGGGGAPGGGGGAAAAAGGGGGGAPDKTHTCPP
CPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFS
CSVMHEALHNHYTQKSLSLSPG,
h) a GLP-2 peptibody comprising the amino acid sequence of
(SEQ ID NO: 22)
HGDGSFSDEMNTILDNLAARDFINWLIQTKITDGGGGGGGDKTHTCPPC
PAPEAAGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKA
LPAPIEKTISKAKGQPREPQVYTLPPSRDELTKNQVSLTCLVKGFYPSD
IAVEWESNGQPENNYKTTPPVLDSDGSFFLYSKLTVDKSRWQQGNVFSC
SVMHEALHNHYTQKSLSLSPG,
i) a GLP-2 peptibody comprising the amino acid sequence of
(SEQ ID NO: 25)
HGDGSFSDEMNTILDNLAARDFINWLIQTKITDGGGGSGGGGSDKTHTC
PPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNV
FSCSVMHEALHNHYTQKSLSLSPG,
and
j) a GLP-2 peptibody comprising the amino acid sequence of
(SEQ ID NO: 28)
HGDGSFSDEMNTILDNLAARDFINWLIQTKITDGGGGGGSGGGGSGGGGS
DAHKSEVAHRFKDLGEENFKALVLIAFAQYLQQCPFEDHVKLVNEVTEFA
KTCVADESAENCDKSLHTLFGDKLCTVATLRETYGEMADCCAKQEPERNE
CFLQHKDDNPNLPRLVRPEVDVMCTAFHDNEETFLKKYLYEIARRHPYFY
APELLFFAKRYKAAFTECCQAADKAACLLPKLDELRDEGKASSAKQRLKC
ASLQKFGERAFKAWAVARLSQRFPKAEFAEVSKLVTDLTKVHTECCHGDL
LECADDRADLAKYICENQDSISSKLKECCEKPLLEKSHCIAEVENDEMPA
DLPSLAADFVESKDVCKNYAEAKDVFLGMFLYEYARRHPDYSVVLLLRLA
KTYKTTLEKCCAAADPHECYAKVFDEFKPLVEEPQNLIKQNCELFEQLGE
YKFQNALLVRYTKKVPQVSTPTLVEVSRNLGKVGSKCCKHPEAKRMPCAE
DYLSVVLNQLCVLHEKTPVSDRVTKCCTESLVNRRPCFSALEVDETYVPK
EFNAETFTFHADICTLSEKERQIKKQTALVELVKHKPKATKEQLKAVMDD
FAAFVEKCCKADDKETCFAEEGKKLVAASRAALGL;
or a pharmaceutically acceptable salt thereof.

108. A GLP-2 peptibody of claim 107, wherein the GLP-2 peptibody comprises the amino acid sequence of
(SEQ ID NO: 1)
HGDGSFSDEMNTILDNLAARDFINWLIQTKITDGGGGGDKTHTCPPCPAP
EAAGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEAL
HNHYTQKSLSLSPG,
or a pharmaceutically acceptable salt thereof.

109. A GLP-2 peptibody of claim 107, wherein the GLP-2 peptibody comprises the amino acid sequence of
(SEQ ID NO: 7)
HGDGSFSDEMNTILDNLAARDFINWLIQTKITDGGGGSGGGGSGGGGSDK
THTCPPCPAPEAAGGPSVFLFPPKPKDTLMISRTPEVTCVVVDVSHEDPE
VKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGNV
FSCSVMHEALHNHYTQKSLSLSPG,
or a pharmaceutically acceptable salt thereof.

110. The pharmaceutical composition of claim 107, which is formulated as a liquid suitable for administration by injection or infusion.

111. A polynucleotide comprising a sequence encoding the GLP-2 precursor polypeptide selected from the group consisting of:
a) a GLP-2 peptibody comprising the amino acid sequence of
(SEQ ID NO: 2)
METPAQLLFLLLLWLPDTTGHGDGSFSDEMNTILDNLAARDFINWLIQTK
ITDGGGGGDKTHTCPPCPAPEAAGGPSVFLFPPKPKDTLMISRTPEVTCV
VVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTV
DKSRWQQGNVFSCSVMHEALHNHYTQKSLSLSPG,
b) a GLP-2 precursor polypeptide comprising the amino acid sequence of
(SEQ ID NO: 5)
METPAQLLFLLLLWLPDTTGHGDGSFSDEMNTILDNLAARDFINWLIQTK
ITDGGGGGDKTHTCPPCPAPEAAGGPSVFLFPPKPKDTLMISRTPEVTCV
VVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTV
DKSRWQQGNVFSCSVMHEALHNHYTQKSLSLSPGK,
c) a GLP-2 precursor polypeptide comprising the amino acid sequence of
(SEQ ID NO: 8)
METPAQLLFLLLLWLPDTTGHGDGSFSDEMNTILDNLAARDFINWLIQTK
ITDGGGGSGGGGSGGGGSDKTHTCPPCPAPEAAGGPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDG
SFFLYSKLTVDKSRWQQGNVFSCSVMHEALHNHYTQKSLSLSPG,
d) a GLP-2 precursor polypeptide comprising the amino acid sequence of
(SEQ ID NO: 11)
METPAQLLFLLLLWLPDTTGGDGSFSDEMNTILDNLAARDFINWLIQTKI
TDGGGGSGGGGSGGGGSDKTHTCPPCPAPEAAGGPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGS
FFLYSKLTVDKSRWQQGNVFSCSVMHEALHNHYTQKSLSLSPGK,
e) a GLP-2 precursor polypeptide comprising the amino acid sequence of
(SEQ ID NO: 14)
METPAQLLFLLLLWLPDTTGHGDGSFSDEMNTILDNLAARDFINWLIQTK
ITDDKTHTCPPCPAPEAAGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPG,
f) a GLP-2 precursor polypeptide comprising the amino acid sequence of
(SEQ ID NO: 17)
METPAQLLFLLLLWLPDTTGHGDGSFSDEMNTILDNLAARDFINWLIQTK
ITDGGGGGGSGGGGSGGGGSDKTHTCPPCPAPEAAGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPG,
g) a GLP-2 precursor polypeptide comprising the amino acid sequence of
(SEQ ID NO: 20)
METPAQLLFLLLLWLPDTTGHGDGSFSDEMNTILDNLAARDFINWLIQTK
ITDGAPGGGGGAAAAAGGGGGGAPGGGGGAAAAAGGGGGGAPGGGGGAAA
AAGGGGGGAPDKTHTCPPCPAPEAAGGPSVFLFPPKPKDTLMISRTPEVT
CVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKL
TVDKSRWQQGNVFSCSVMHEALHNHYTQKSLSLSPG,
h) a GLP-2 precursor polypeptide comprising the amino acid sequence of
(SEQ ID NO: 23)
METPAQLLFLLLLWLPDTTGHGDGSFSDEMNTILDNLAARDFINWLIQTK
ITDGGGGGGGDKTHTCPPCPAPEAAGGPSVFLFPPKPKDTLMISRTPEVT
CVVVDVSHEDPEVKFNWYVDGVEVHNAKTKPREEQYNSTYRVVSVLTVLH
QDWLNGKEYKCKVSNKALPAPIEKTISKAKGQPREPQVYTLPPSRDELTK
NQVSLTCLVKGFYPSDIAVEWESNGQPENNYKTTPPVLDSDGSFFLYSKL
TVDKSRWQQGNVFSCSVMHEALHNHYTQKSLSLSPG,
i) a GLP-2 precursor polypeptide comprising the amino acid sequence of
(SEQ ID NO: 26)
METPAQLLFLLLLWLPDTTGHGDGSFSDEMNTILDNLAARDFINWLIQTK
ITDGGGGSGGGGSDKTHTCPPCPAPEAAGGPSVFLFPPKPKDTLMISRTP
EVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLY
SKLTVDKSRWQQGNVFSCSVMHEALHNHYTQKSLSLSPG,
and
j) a GLP-2 precursor polypeptide comprising the amino acid sequence of
(SEQ ID NO: 29)
METPAQLLFLLLLWLPDTTGHGDGSFSDEMNTILDNLAARDFINWLIQTK
ITDGGGGGGSGGGGSGGGGSDAHKSEVAHRFKDLGEENFKALVLIAFAQY
LQQCPFEDHVKLVNEVTEFAKTCVADESAENCDKSLHTLFGDKLCTVATL
RETYGEMADCCAKQEPERNECFLQHKDDNPNLPRLVRPEVDVMCTAFHDN
EETFLKKYLYEIARRHPYFYAPELLFFAKRYKAAFTECCQAADKAACLLP
KLDELRDEGKASSAKQRLKCASLQKFGERAFKAWAVARLSQRFPKAEFAE
VSKLVTDLTKVHTECCHGDLLECADDRADLAKYICENQDSISSKLKECCE
KPLLEKSHCIAEVENDEMPADLPSLAADFVESKDVCKNYAEAKDVFLGMF
LYEYARRHPDYSVVLLLRLAKTYKTTLEKCCAAADPHECYAKVFDEFKPL
VEEPQNLIKQNCELFEQLGEYKFQNALLVRYTKKVPQVSTPTLVEVSRNL
GKVGSKCCKHPEAKRMPCAEDYLSVVLNQLCVLHEKTPVSDRVTKCCTES
LVNRRPCFSALEVDETYVPKEFNAETFTFHADICTLSEKERQIKKQTALV
ELVKHKPKATKEQLKAVMDDFAAFVEKCCKADDKETCFAEEGKKLVAASR
AALGL.

112. A polynucleotide comprising a sequence encoding the GLP-2 precursor polypeptide comprising the amino acid sequence of
(SEQ ID NO: 1)
METPAQLLFLLLLWLPDTTGHGDGSFSDEMNTILDNLAARDFINWLIQTK
ITDGGGGGDKTHTCPPCPAPEAAGGPSVFLFPPKPKDTLMISRTPEVTCV
VVDVSHEDPEVKFNWYVDGVEVHNAKTKPREEQYNSTYRVVSVLTVLHQD
WLNGKEYKCKVSNKALPAPIEKTISKAKGQPREPQVYTLPPSRDELTKNQ
VSLTCLVKGFYPSDIAVEWESNGQPENNYKTTPPVLDSDGSFFLYSKLTV
DKSRWQQGNVFSCSVMHEALHNHYTQKSLSLSPG.

113. The polynucleotide of claim 112, wherein the sequence encoding the GLP-2 peptibody comprises the polynucleotide sequence of SEQ ID NO: 3.

114. A polynucleotide comprising a sequence encoding a GLP-2 precursor polypeptide comprising the amino acid sequence of
(SEQ ID NO: 8)
METPAQLLFLLLLWLPDTTGHGDGSFSDEMNTILDNLAARDFINWLIQTK
ITDGGGGSGGGGSGGGGSDKTHTCPPCPAPEAAGGPSVFLFPPKPKDTLM
ISRTPEVTCVVVDVSHEDPEVKFNWYVDGVEVHNAKTKPREEQYNSTYRV
VSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDG
SFFLYSKLTVDKSRWQQGNVFSCSVMHEALHNHYTQKSLSLSPG.

115. The polynucleotide of claim 114, wherein the sequence encoding the GLP-2 precursor polypeptide comprises the polynucleotide sequence of SEQ ID NO: 9.

116. A vector comprising the polynucleotide of claim 114.

117. A method for treating a patient with enterocutaneous fistula (ECF) comprising treating said patient with the GLP-2 peptibody of claim 107 using a dosing regimen effective to promote closure, healing, and/or repair of the ECF.

118. A method for treating a patient with obstructive jaundice comprising treating said patient with the GLP-2 peptibody of claim 107 using a dosing regimen effective to promote closure, healing, and/or repair of the obstructive jaundice.

119. A method for treating or preventing radiation damage to the gastrointestinal tract of a patient comprising treating said patient with the GLP-2 peptibody of claim 107 using a dosing regimen effective to treat or prevent radiation damage to the gastrointestinal tract of a patient.

120. A method for treating a patient with short bowel syndrome presenting with colon in continuity with remnant small intestine comprising treating said patient with the GLP-2 peptibody of claim 107 using a dosing regimen effective to treat said short bowel syndrome.
